SEQUENCE LISTING (91 sequences)

SEQ ID NO: 1 - DNA, 2713 bp, Homo sapiens
ggcacgaggagagtgcggctgctgagagccgagcccagcaatcccgatcctctgagtcgtgaagaagggaggcagcgagggggttggggttggggcctgaggcaagcccccaggctccgctcttgccagagggacaggagccatggctcagaaaatggactgtggtgcgggcctcctcggcttccaggctgaggcctccgtagaagacagcgccttgcttatgcagaccttgatggaggccatccagatctcagaggctccacctactaaccaggccaccgcagctgctagtccccagagttcacagcccccaactgccaatgagatggctgacattcaggtttcagcagctgccgctaggcctaagtcagcctttaaagtccagaatgccaccacaaaaggcccaaatggtgtctatgatttctctcaggctcataatgccaaggatgtgcccaacacgcagcccaaggcagcctttaagtcccaaaatgctacctccaaaggtccaaatgctgcctatgatttttcccaggcagcaaccactggtgagttagctgctaacaagtctgagatggccttcaaggcccagaatgccactactaaagtgggcccaaatgccacctacaatttctctcagtctctcaatgccaatgacctggccaacagcaggcctaagacccctttcaaggcttggaatgataccactaaggccccaacagctgatacccagacccagaatgtaaatcaggccaaaatggccacttcccaggctgacatagagaccgacccaggtatctctgaacctgacggtgcaactgcacagacatcagcagatggttcccaggctcagaatctggagtcccggacaataattcggggcaagaggacccgcaagattaataacttgaatgttgaagagaacagcagtggggatcagaggcgggccccactggctgcagggacctggaggtctgcaccagttccagtgaccactcagaacccacctggcgcaccccccaatgtgctctggcagacgccattggcttggcagaacccctcaggctggcaaaaccagacagccaggcagaccccaccagcacgtcagagccctccagctaggcagaccccaccagcctggcagaacccagtcgcttggcagaacccagtgatttggccaaacccagtaatctggcagaacccagtgatctggccaaaccccattgtctggcccggccctgttgtctggccgaatccactggcctggcagaatccacctggatggcagactccacctggatggcagaccccaccgggctggcagggtcctccagactggcaaggtcctcctgactggccgctaccacccgactggccactgccacctgattggccacttcccactgactggccactaccacctgactggatccccgctgattggccaattccacctgactggcagaacctgcgcccctcgcctaacctgcgcccttctcccaactcgcgtgcctcacagaacccaggtgctgcacagccccgagatgtggcccttcttcaggaaagagcaaataagttggtcaagtacttgatgcttaaggactacacaaaggtgcccatcaagcgctcagaaatgctgagagatatcatccgtgaatacactgatgtttatccagaaatcattgaacgtgcatgctttgtcctagagaagaaatttgggattcaactgaaagaaattgacaaagaagaacacctgtatattctcatcagtacccccgagtccctggctggcatactgggaacgaccaaagacacacccaagctcggtctcctcttggtgattctgggtgtcatcttcatgaatggcaaccgtgccagtgaggctgtcctctgggaggcactacgcaagatgggactgcgtcctggggtgagacatcccctccttggagatctaaggaaacttctcacctatgagtttgtaaagcagaaatacctggactacagacgagtgcccaacagcaaccccccggagtatgagttcctctggggcctccgttcctaccatgagactagcaagatgaaagtgctgagattcattgcagaggttcagaaaagagaccctcgtgactggactgcacagttcatggaggctgcagatgaggccttggatgctctggatgctgctgcagctgaggccgaagcccgggctgaagcaagaacccgcatgggaattggagatgaggctgtgtctgggccctggagctgggatgacattgagtttgagctgctgacctgggatgaggaaggagattttggagatccctggtccagaattccatttaccttctgggccagataccaccagaatgcccgctccagattccctcagacctttgccggtcccattattggtcctggtggtacagccagtgccaacttcgctgccaactttggtgccattggtttcttctgggttgagtgagatgttggatattgctatcaatcgcagtagtctttcccctgtgtgagctgaagcctcagattccttctaaacacagctatctagagagccacatcctgttgactgaaagtggcatgcaagataaatttatttgctgttccttgtctactgctttttttccccttgtgtgctgtcaagttttggtatcagaaataaacattgaaattgcaaagtgaaaaaaaaaaaaaaaaaaaa

SEQ ID NO: 2 - DNA, 642 bp, Homo sapiens
atgtccgagactgctcctgccgctcccgctgccgcgcctcctgcggagaaggcccctgtaaagaagaaggcggccaaaaaggctgggggtacgcctcgtaaggcgtccggtcccccggtgtcagagctcatcaccaaggctgtggccgcctctaaagagcgtagcggagtttctctggctgctctgaaaaaagcgttggctgccgccggctatgatgtggagaaaaacaacagccgtatcaaacttggtctcaagagcctggtgagcaagggcactctggtgcaaacgaaaggcaccggtgcttctggctcctttaaactcaacaagaaggcagcctccggggaagccaagcccaaggttaaaaaggcgggcggaaccaaacctaagaagccagttggggcagccaagaagcccaagaaggcggctggcggcgcaactccgaagaagagcgctaagaaaacaccgaagaaagcgaagaagccggccgcggccactgtaaccaagaaagtggctaagagcccaaagaaggccaaggttgcgaagcccaagaaagctgccaaaagtgctgctaaggctgtgaagcccaaggccgctaagcccaaggttgtcaagcctaagaaggcggcgcccaagaagaaatag

SEQ ID NO: 3 - DNA, 542 bp, Homo sapiens
gtctgccctctctgctcgccctgcctagcttgaggatctgtcaccccagccatgaggattatcgccctcctcgctgctattctcttggtagccctccaggtccgggcaggcccactccaggcaagaggtgatgaggctccaggccaggagcagcgtgggccagaagaccaggacatatctatttcctttgcatgggataaaagctctgctcttcaggtttcaggctcaacaaggggcatggtctgctcttgcagattagtattctgccggcgaacagaacttcgtgttgggaactgcctcattggtggtgtgagtttcacatactgctgcacgcgtgtcgattaacgttctgctgtccaagagaatgtcatgctgggaacgccatcatcggtggtgttagcttcacatgcttctgcagctgagcttgcagaatagagaaaaatgagctcataatttgctttgagagctacaggaaatggttgtttctcctatactttgtccttaacatctttcttgatcctaaatatatatctcgtaacaag

SEQ ID NO: 4 - DNA, 2856 bp, Homo sapiens
tagtcgcgggtccccgagtgagcacgccagggagcaggagaccaaacgacgggggtcggagtcagagtcgcagtgggagtccccggaccggagcacgagcctgagcgggagagcgccgctcgcacgcccgtcgccacccgcgtacccggcgcagccagagccaccagcgcagcgctgccatggagcccagcagcaagaagctgacgggtcgcctcatgctggctgtgggaggagcagtgcttggctccctgcagtttggctacaacactggagtcatcaatgccccccagaaggtgatcgaggagttctacaaccagacatgggtccaccgctatggggagagcatcctgcccaccacgctcaccacgctctggtccctctcagtggccatcttttctgttgggggcatgattggctccttctctgtgggccttttcgttaaccgctttggccggcggaattcaatgctgatgatgaacctgctggccttcgtgtccgccgtgctcatgggcttctcgaaactgggcaagtcctttgagatgctgatcctgggccgcttcatcatcggtgtgtactgcggcctgaccacaggcttcgtgcccatgtatgtgggtgaagtgtcacccacagcctttcgtggggccctgggcaccctgcaccagctgggcatcgtcgtcggcatcctcatcgcccaggtgttcggcctggactccatcatgggcaacaaggacctgtggcccctgctgctgagcatcatcttcatcccggccctgctgcagtgcatcgtgctgcccttctgccccgagagtccccgcttcctgctcatcaaccgcaacgaggagaaccgggccaagagtgtgctaaagaagctgcgcgggacagctgacgtgacccatgacctgcaggagatgaaggaagagagtcggcagatgatgcgggagaagaaggtcaccatcctggagctgttccgctcccccgcctaccgccagcccatcctcatcgctgtggtgctgcagctgtcccagcagctgtctggcatcaacgctgtcttctattactccacgagcatcttcgagaaggcgggggtgcagcagcctgtgtatgccaccattggctccggtatcgtcaacacggccttcactgtcgtgtcgctgtttgtggtggagcgagcaggccggcggaccctgcacctcataggcctcgctggcatggcgggttgtgccatactcatgaccatcgcgctagcactgctggagcagctaccctggatgtcctatctgagcatcgtggccatctttggctttgtggccttctttgaagtgggtcctggccccatcccatggttcatcgtggctgaactcttcagccagggtccacgtccagctgccattgccgttgcaggcttctccaactggacctcaaatttcattgtgggcatgtgcttccagtatgtggagcaactgtgtggtccctacgtcttcatcatcttcactgtgctcctggttctgttcttcatcttcacctacttcaaagttcctgagactaaaggccggaccttcgatgagatcgcttccggcttccggcaggggggagccagccaaagtgataagacacccgaggagctgttccatcccctgggggctgattcccaagtgtgagtcgccccagatcaccagcccggcctgctcccagcagccctaaggatctctcaggagcacaggcagctggatgagacttccaaacctgacagatgtcagccgagccgggcctggggctcctttctccagccagcaatgatgtccagaagaatattcaggacttaacggctccaggattttaacaaaagcaagactgttgctcaaatctattcagacaagcaacaggttttataatttttttattactgattttgttatttttatatcagcctgagtctcctgtgcccacatcccaggcttcaccctgaatggttccatgcctgagggtggagactaagccctgtcgagacacttgccttcttcacccagctaatctgtagggctggacctatgtcctaaggacacactaatcgaactatgaactacaaagcttctatcccaggaggtggctatggccacccgttctgctggcctggatctccccactctaggggtcaggctccattaggatttgccccttcccatctcttcctacccaaccactcaaattaatctttctttacctgagaccagttgggagcactggagtgcagggaggagaggggaagggccagtctgggctgccgggttctagtctcctttgcactgagggccacactattaccatgagaagagggcctgtgggagcctgcaaactcactgctcaagaagacatggagactcctgccctgttgtgtatagatgcaagatatttatatatatttttggttgtcaatattaaatacagacactaagttatagtatatctggacaagccaacttgtaaatacaccacctcactcctgttacttacctaaacagatataaatggctggtttttagaaacatggttttgaaatgcttgtggattgagggtaggaggtttggatgggagtgagacagaagtaagtggggttgcaaccactgcaacggcttagacttcgactcaggatccagtcccttacacgtacctctcatcagtgtcctcttgctcaaaaatctgtttgatccctgttacccagagaatatatacattctttatcttgacattcaaggcatttctatcacatatttgatagttggtgttcaaaaaaacactagttttgtgccagccgtgatgctcaggcttgaaatcgcattattttgaatgtgaagggaa

SEQ ID NO: 5 - DNA, 4461 bp, Homo sapiens
cttgttgttgatccgtacccagtgggcagcgccgggagctggaccaagcggccggtgagaggccgctgtagcggtgctcagccacctgtgctgcctgccagggggcgggccgaaacctggaggcccggggggcccagctcccgtagggagccgtgggcgctcggtgcccgggccgggcaggacagaataataagctgaatagaatctgaccattggctttcacctggccaggaccttctatgtagctctccttttgtggcccatgtgctgcatcctctgccctcagtgtgcaactggcccccaacgcaatgtgtgtttgtcaaaccatggaagtggggcagtatggcaagaatgcaagtcgggctggagaccggggagtcctcctggagcccttcatccaccaagtaggcggacacagcagcatgatgcgttacgacgatcacactgtgtgcaagcccctcatctcccgggaacagcgcttttacgagtccctccctcccgaaatgaaggagttcacccctgaatacaaaggcgtggtatctgtctgttttgagggggacagtgatggttacatcaacttagtggcctatccttatgtggaaagtgagactgtggaacaggatgacacaacagaacgggagcaacctcggcgcaaacactcccgccggagcctgcaccggtcaggcagtggcagtgaccacaaggaggagaaagccagcctgtcccttgagacctctgagagctcacaggaggcaaagagtccgaaggtggagctgcacagccactcagaggtccctttccagatgctagatggcaacagtggcttgagttctgagaagatcagccacaacccctggagcctgcgttgtcacaagcagcagctgagccgcatgcgctccgagtccaaggaccgaaagctctacaagttcctcctgcttgagaacgtggtgcaccacttcaagtacccctgcgtgttggacctgaagatgggcacgcggcagcatggcgatgacgcgtcagctgagaaggcagcccggcagatgcggaaatgcgagcagagcacatcagccacgctgggcgtcagggtctgcggcatgcaggtgtaccagctggacacagggcattacctctgcaggaacaagtactatggccgtgggctctccattgaaggcttccgcaatgccctctatcaatatctgcacaatggcctggacctgcgacgtgacctgtttgagcctatcctgagcaaactgcggggcctgaaagctgtgctggagcggcaggcctcttaccgcttctactccagttccctgcttgtcatctatgatggcaaggagtgccgggctgagtcctgcctggaccgccggtctgagatgcgtctcaagcacctggacatggtgctccctgaggtggcgtcatcctgtggccccagcaccagccccagcaacaccagccccgaggcgggtccctcctctcagcccaaggtggatgtccgcatgattgactttgcacacagcacattcaagggcttccgggatgaccccaccgtgcatgatgggccagacagaggctacgtgtttggcctggagaacctcatcagcatcatggaacagatgcgggacgagaaccagtaggccctgttctgggcccccagaaccccttcctctccactgcaggcagggaccattgttctgaacttgccgtgaggacacacagacttgcttttaaagggttatatttctctttggtgtaaactaaaagaaatgtttttagctgtagcctggaatccatatatataaagtgaaggagggcagaccacacgccctctcagccaggctcctcagctttgtggctctgactggtgtgtccaggctgccttaggaaggaagaggtgcccctggtgggcttggcagcagggacagggtgcccttggacattggtttctcttgtctagatctttgagatctgtggctgcagggccctgctgattgtaaggtaaagccctgggctggtgcagggcccctccacgcccactcttcccttgttccccagaagtagagggctctgggtgcccatttcttgggggctttccagtcttatgctgtgggtgtcagctagctctttaataggtgccctcagggcaccacagggctgactgcacaaagctggacccatccttcggtctgaccttagcatggggctagattaatgaagctgggctgaggccaacttatggcagagggcggcgcctgggttccccaggcacctgttggcacgtgacaggttggcacctgtcctattcctgaaacagcctctctcaccaagttcccttgcctaagaaggccactccctcccaccccactgaagtgggggatagtcggtgtcctagcaggcctcagggcctctggtggctctggcccagacagtatttgcagttcttgtgctatgggtgggagtcttcttcctcaagtttcggcagctgtgctgctgctggatgggctgctcctcccagggctcaagggctgtggtccgctcagggtctcatttccccaggccaagttcaaggcagcagccctttgtgaggcgctcttggccctgggcctggagggagaactttaagcttttttgctcacagggacgtggtatgggccctgggtgcaggtgcccacattctgctaatgagagctttgtctgatcagtcctgggtccatcagtttgtccatgtgtccggctgccagcccgtcccttgggatccttcccctggggtgtagccttgttcattagtatatactcattccttcatgctttcctcagcagaacacttccacttctgaggtgagcttttgccccgtgcccttcctccacaggtgttgcctttttataaagacctgatagcagaataaattggtgtttccctgttgacccagcaccatttctgtgggcctagaatatggccctcaacccttagagtggggcagtgagggcttgaggagtgacccttcctttctcatggttttagtcattttggctgccagcccttaatggcacagatctgctgcttctaacagatggccaggaggtgacaccgatttcagccattgccaaggttagcaccctctcctttgagcctagggccacactgttcattgtcactttaggcaagtgcctgtttggctttaaaggtaagcctgccagctgtgagaagccttggtaactgatggactcatttcctggtccttaaagatgcagcctcttaagggctccttgatggatgccatctctcctagcccccagccctggtgccactggtgggcaggttcccattctttggggctgggagggacagcttgcctgtttctggtcacaaattacagtcttctctcctgtaccattctgtggcttcagccatgggggcagtagcccttcattagtgtagatagtcattccctggtagggtggagggtaagacatagggtctggaactgtttgggaccttttggggatgtcctgtgcctcccagattcctagattctgggaggagaggctgccgcattctgctgctcctcacagcgagcaaagctgcacccacttacattcagtattttcctggcactacaaagagtgggaaggcctgggatttgctgctgctcccttagagcagggcccctcttttcagcactttggacacctggagacccagccctgttatttaatggtagtgggcaagtgtgtgtgcatactgtctgccactgctttctccctgccccatgccagagagccctgtccctgccaggcccagccttcttagccccaacttgggaacaaagtgcaacatgggatcatgggttggggtgctcaggtgagccctctctatagtgcttccctgggccaagctgacaccagcccctgagggtggggtgggacgggtggtgcttaaaagaggaaggggaccagtgtagcaacttgccagggaccccacccctccctctctgggcctgtgcagtgagcatggggattcccatcaaggggcctggcacctgtgctagttacgtagccgctgctcacgcgctcactcctgaccacatgcacgttccctagatgcagactgctttgaactttaaagctgtacaatttggttatgtttgtgctgacttaaaatatattttaatgaggaaaaaataatggagaaccctgggaaggacctggttcttttgcttctcggggaactgtaagccctcgcgttctgggaatcgctctctgctgctctttcctggaagctaagcctgtctccaccgcccgaggcctgcgccggtggctcccgccgcagttgcgtttgctttggaccttgcgtgcgggggagggggtgctcggtccgagcccgctcctttctgtacacctagcgctgcccgccccgcttgtgtctgaggtcgtgtatgtcaaaaataaagccgctagaaacgg

SEQ ID NO: 6 - DNA, 847 bp, Homo sapiens
ggccacatggactggggtgcaatgggacagctgctgccagcgagagggaccagggcaccactctctagggagcccacactgcaagtcaggccacaaggacctctgaccctgagggccgatgaggccagggacaggccaggggggccttgaggcccctggtgagccaggccccaacctcaggcagcgctggcccctgctgctgctgggtctggccgtggtaacccatggcctgctgcgcccaacagctgcatcgcagagcagggccctgggccctggagcccctggaggaagcagccggtccagcctgaggagccggtggggcaggttcctgctccagcgcggctcctggactggccccaggtgctggccccgggggtttcaatccaagcataactcagtgacgcatgtgtttggcagcgggacccagctcaccgttttaagtcagcccaaggccaccccctcggtcactctgttcccgccgtcctctgaggagctccaagccaacaaggctacgctggtgtgtctcatgaatgacttttatccgggaatcttgacggtgacctggaaggcagatggtacccccatcacccagggcgtggagatgaccacgccctccaaacagagcaacaacaagtacgcggccagcagctacctgagcctgacgcccgagcagtggaggtcccgcagaagctacagctgccaggtcatgcacgaagggagcaccgtggagaagacggtggcccctgcagaatgttcataggttcccagccccgaccccacccaaaggcctggagctgcaggatcccaggggaagggtctctctctgcatcccaagccatccagccct

SEQ ID NO: 7 - DNA, 2489 bp, Homo sapiens
attaccaggcacgcgcaggaaacatggcggcggcgggtgttgtgagcgggaagattatatatgaacaagaaggagtatatattcactcatcttgtggaaagaccaatgaccaagacggcttgatttcaggaatattacgtgttttagaaaaggatgccgaagtaatagtggactggggaccattggatgatgcattagattcctctagtattctctatgctagaaaggactccagttcagttgtagaatggactcaggccccaaaagaaagaggtcatcgaggatcagaacatctgaacagttacgaagcagaatgggacatggttaatacagtttcatttaaaaggaaaccacataccaatggagatgctccaagtcatagaaatgggaaaagcaaatggtcattcctgttcagtttgacagacctgaaatcaatcaagcaaaacaaagagggtatgggctggtcctatttggtattctgtctaaaggatgacgtcgttctccctgctctacactttcatcaaggagatagcaaactactgattgaatctcttgaaaaatatgtggtattgtgtgaatctccacaggataaaagaacacttcttgtgaattgtcagaataagagtctttcacagtcttttgaaaatcttcttgatgagccagcatatggtttaatacaaaaaattaaaaaggacccttatacggcaactatgataggattttccaaagtcacaaactacatttttgacagtttgagaggcagcgatccctctacacatcaacgaccaccttcagaaatggcagattttcttagtgatgctattccaggtctaaagataaatcaacaagaagaaccaggatttgaagtcatcacaagaattgatttgggggaacgccctgttgttcaaaggagagaaccggtatcactggaagaatggactaagaacattgattctgaaggaagaattttaaatgtagataatatgaagcagatgatatttagagggggacttagtcatgcattgagaaagcaagcatggaaatttcttctgggttattttccctgggacagtaccaaggaggaaagaacccaattacaaaagcaaaaaactgatgaatacttcagaatgaaactgcagtggaaatccatcagccaggaacaagagaaaagaaattcgaggttaagagattatagaagtcttatcgaaaaagatgttaacagaacagatcgaacaaacaagttttatgaaggccaagataatccagggttgattttacttcatgacattttgatgacctactgtatgtatgattttgatttaggatatgttcagggaatgagtgatttactttcccctcttttatatgtgatggaaaatgaagtggatgccttttggtgctttgcctcttacatggaccaaatgcatcagaattttgaagaacaaatgcaaggcatgaagacccagctaattcagctgagtaccttacttcgattgttagacagtggattttgcagttacttagaatctcaggactctggatacctttatttttgcttcaggtggcttttaatcagattcaaaagggaatttagttttctagatattcttcgattatgggaggtaatgtggaccgaactaccatgtacaaatttccatcttcttctctgttgtgctattctggaatcagaaaagcagcaaataatggaaaagcattatggcttcaatgaaatacttaagcatatcaatgaattgtccatgaaaattgatgtggaagatatactctgcaaggcagaagcaatttctctacagatggtaaaatgcaaggaattgccacaagcagtctgtgagatccttgggcttcaaggcggtgaagttacaacaccagattcagacgttggtgaagacgaaaatgttgtcatgactccttgtcctacatctgcatttcaaagtaatgccttgcctacactctctgccagtggagccagaaatgacagcccaacacagataccagtgtcctcagatgtctgcagattaacacctgcatgatcactgttcttgcttttttgggaagagacactttgttgcaaccctttttcaagtacttgaaagttgaaaatttgaaatcttggtattgatcatgctttaaggtttatgtaaagaaagtgtactgatgttcttacattaaagctttacaaagatttaaactaattatttttgtagttacttctaccaaatagcctttccttttcgataacattcctcagtatttttatagccaagtacattttattttcttgctgatgaactggaattggataaatattgcaagtggatgagttggaaattatgcactttgaaaaacattcactttgtttaagcttattgggtttcagatttgattaaattaaatgtggaggctttctatagcattctaagctgagaagtagattgttacccagtaatgaaataaaaaataaaaataaaagg

SEQ ID NO: 8 - DNA, 1673 bp, Homo sapiens
agcccagcactagaagtcggcggtgtttccattcggtgatcagcactgaacacagaggactcaccatggagtttgggctgagctgggttttcctcgttgctcttttaagaggtgtccagtgtcaggtgcagctggtggagtctgggggaggcgtggtccagcctgggaggtccctgagactctcctgtgcagcgtctggattcaccttcagtaattatggcatgcactgggtccgccaggctccaggcaaggggctggagtgggtggcagctatatggtatgatggaagtaataaatactatgcagactccgtgaagggccgattcaccatctccagagacaattccaagaacacgttgtatatgcaaatgaacagcctgagagccgaggacacggctgtgtattattgtgcgagagagggtcggtgggtacgatatactacggtgactactatcggatactactttgactactggggccagggaaccctggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaatgagtgcgacggccggcaagcccccgctccccgggctctcgcggtcgcacgaggatgcttggcacgtaccccgtctacatacttcccaggcacccagcatggaaataaagcacccaccactgccctgggccctgcaaaaaaaaaaaaaaaaaaaaaaaaaaaaaaaaaaaaaaaaaa

SEQ ID NO: 9 - DNA, 1264 bp, Homo sapiens
gtggtacccagtcctcaggtgcaaccccctgcgtggtcctctgtggcagccttctctcattcagagctgttttccacagaggtagtgaaaagaactggattttcaagttcactttgcaagagaaaaagaaaactcagtagaagataatggcaagtccagactggggatatgatgacaaaaatggtcctgaacaatggagcaagctgtatcccattgccaatggaaataaccaatcccctgttgatattaaaaccagtgaaaccaaacatgacacctctctgaaacctattagtgtctcctacaacccagccacagccaaagaaattatcaatgtggggcattctttccatgtaaattttgaggacaacgataaccgatcagtgctgaaaggtggtcctttctctgacagctacaggctctttcagtttcattttcactggggcagtacaaatgagcatggttcagaacatacagtggatggagtcaaatattctgccgagcttcacgtagctcactggaattctgcaaagtactccagccttgctgaagctgcctcaaaggctgatggtttggcagttattggtgttttgatgaaggttggtgaggccaacccaaagctgcagaaagtacttgatgccctccaagcaattaaaaccaagggcaaacgagccccattcacaaattttgacccctctactctccttccttcatccctggatttctggacctaccctggctctctgactcatcctcctctttatgagagtgtaacttggatcatctgtaaggagagcatcagtgtcagctcagagcagctggcacaattccgcagccttctatcaaatgttgaaggtgataacgctgtccccatgcagcacaacaaccgcccaacccaacctctgaagggcagaacagtgagagcttcattttgatgattctgagaagaaacttgtccttcctcaagaacacagccctgcttctgacataatccagttaaaataataatttttaagaaataaatttatttcaatattagcaagacagcatgccttcaaatcaatctgtaaaactaagaaacttaaattttagttcttactgcttaattcaaataataattagtaagctagcaaatagtaatctgtaagcataagcttatcttaaattcaagtttagtttgaggaattctttaaaattacaactaagtgatttgtatgtctatttttttcagtttatttgaaccaataaaataattttatctctttct

SEQ ID NO: 10 - DNA, 2454 bp, Homo sapiens
ggaataggttagtttcagacaagcctgcttgccggagctcagcagacaccaggccttccgggcaggcctggcccaccgtgggcctcagagctgctgctggggcattcagaaccggctctccattggcattgggaccagagaccccgcaagtggcctgtttgcctggacatccacctgtacgtccccaggtttcgggaggcccaggggcgatgccagaccccgcggcgcacctgcccttcttctacggcagcatctcgcgtgccgaggccgaggagcacctgaagctggcgggcatggcggacgggctcttcctgctgcgccagtgcctgcgctcgctgggcggctatgtgctgtcgctcgtgcacgatgtgcgcttccaccactttcccatcgagcgccagctcaacggcacctacgccattgccggcggcaaagcgcactgtggaccggcagagctctgcgagttctactcgcgcgaccccgacgggctgccctgcaacctgcgcaagccgtgcaaccggccgtcgggcctcgagccgcagccgggggtcttcgactgcctgcgagacgccatggtgcgtgactacgtgcgccagacgtggaagctggagggcgaggccctggagcaggccatcatcagccaggccccgcaggtggagaagctcattgctacgacggcccacgagcggatgccctggtaccacagcagcctgacgcgtgaggaggccgagcgcaaactttactctggggcgcagaccgacggcaagttcctgctgaggccgcggaaggagcagggcacatacgccctgtccctcatctatgggaagacggtgtaccactacctcatcagccaagacaaggcgggcaagtactgcattcccgagggcaccaagtttgacacgctctggcagctggtggagtatctgaagctgaaggcggacgggctcatctactgcctgaaggaggcctgccccaacagcagtgccagcaacgcctcaggggctgctgctcccacactcccagcccacccatccacgttgactcatcctcagagacgaatcgacaccctcaactcagatggatacacccctgagccagcacgcataacgtccccagacaaaccgcggccgatgcccatggacacgagcgtgtatgagagcccctacagcgacccagaggagctcaaggacaagaagctcttcctgaagcgcgataacctcctcatagctgacattgaacttggctgcggcaactttggctcagtgcgccagggcgtgtaccgcatgcgcaagaagcagatcgacgtggccatcaaggtgctgaagcagggcacggagaaggcagacacggaagagatgatgcgcgaggcgcagatcatgcaccagctggacaacccctacatcgtgcggctcattggcgtctgccaggccgaggccctcatgctggtcatggagatggctgggggcgggccgctgcacaagttcctggtcggcaagagggaggagatccctgtgagcaatgtggccgagctgctgcaccaggtgtccatggggatgaagtacctggaggagaagaactttgtgcaccgtgacctggcggcccgcaacgtcctgctggttaaccggcactacgccaagatcagcgactttggcctctccaaagcactgggtgccgacgacagctactacactgcccgctcagcagggaagtggccgctcaagtggtacgcacccgaatgcatcaacttccgcaagttctccagccgcagcgatgtctggagctatggggtcaccatgtgggaggccttgtcctacggccagaagccctacaagaagatgaaagggccggaggtcatggccttcatcgagcagggcaagcggatggagtgcccaccagagtgtccacccgaactgtacgcactcatgagtgactgctggatctacaagtgggaggatcgccccgacttcctgaccgtggagcagcgcatgcgagcctgttactacagcctggccagcaaggtggaagggcccccaggcagcacacagaaggctgaggctgcctgtgcctgagctcccgctgcccaggggagccctccacgccggctcttccccaccctcagccccaccccaggtcctgcagtctggctgagccctgcttggttgtctccacacacagctgggctgtggtagggggtgtctcaggccacaccggccttgcattgcctgcctggccccctgtcctctctggctggggagcagggaggtccgggagggtgcggctgtgcagcctgtcctgggctggtggctcccggagggccctgagctgagggcattgcttacacggatgccttcccctgggccctgacattggagcctgggcatcctcaggtggtcaggcgtagatcaccagaataaacccagcttccctcttgaaaaaaaaaaaaaaaaaaaaacc

SEQ ID NO: 11 - DNA, 2196 bp, Homo sapiens
agatctcctgaggtcaggagttcaagacaagcccagacaacttggtgaatgaaaccccatctctactaaaaacaaaaacagaaacaacaaaaaagaaagagccctctggttaaccttgtatgtgtgagacgattatgatgagatagatcccagattgaacaactggtcacccaggaattttaaatttgctgctggagggcacaaaattttgtctctctttcctttttcttacactgggctcttggctctaaatgtagaggctcacatcattctccctgtgaggcgcttggacagagagctcttatgctgttcactcaccaggtgccaaggcagagtagattctaatatttgagttgaacattcttgaacagttatcctgggaaacagtagataccagacagcccttgaactggctccaggccgctttttatttgcaggctctcagttcagcagtgcttgtggggatgggcctgtttcatactctagattgactgggagggaatcaagccagatggcattcacctcccagagatgtatcctagacacacatttccacattgtcagggttctggtgctttcttacagtcatgccctacacagtgtgtccctacaaaaggtccgaactttcaccttcagatccttcttcccttgattgtgggcaaacttggctgaatctagttctgttttattccaaaggacaatttatatcacattgttcacagaagagacattccccctgccccgtcaaccttttccacaccactgcacccaccaggtgatttgcatattgtcccctagggtggacccttccccttgtgagtctgagataaaaagctcagctctatccttgccttgactgatcaggactcctcagttcaccttctcaccatgaggctccctgctcagctcctggggctgctaatgctctgggtccctggtaaggacagaaagagatgagggaggacaactgggtgggaggtgagctctgtgggctccacagcttcacatgtttattccaataatgtgatagaggcacatggtctatgctccagggaatggaattcaggtttgtcttatgaataatcaggattcacctccagggaacgatgaccagtgctctgattaagaacttgaaaaaaaagagttcccttgtggctaataaataatgggtctattttagaaagtctacttttcatgatataaatcaaaactttaaaaatgtaactgtaaatttatatcacaagagaaattatgaaagttgctcataatgtatctatataaacttgcacttctctgttattatttcaggatccagtgaggatattgtgatgacccagactccactctccctgcccgtcacccctggagagccggcctccatctcctgcaggtctagtcagagcctcttggatagtgatgatggaaacacctatttggactggtacctgcagaagccagggcagtctccacagctcctgatctatacgctttcctatcgggcctctggagtcccagacaggttcagtggcagtgggtcaggcactgatttcacactgaaaatcagcagggtggaggctgaggatgttggagtttattactgcatgcaacgtatagagtttccttccacagtggtacagccctgaacagaaacctccctgctgtggtgccccagctgctcacatgcactgcttgtctggggagcaggtcagcagcgtctctgagtctgcaaaagaggaggctgttggagaatacagggcagggtttgcttctgaggactctgcctgggactacaggtgcatgccactaaacatggctaatttttctatttttttgtagagtcggtgcttcaccatgttgcccagcctgttgtcaaaatcatgggctcaagccacccacctgacttggcctcccaacgtgctggcagtacagtgtgagccactgcggcaggtcagcacccctgtttatgttcctgtcacctgccacagccttgactctcataaccaacaggaaaatgaggaggttctagggccctgtgagtaaaaaactgggatgatagggaaaggagaatggaatctcatctgaatcctccttccttgcctacatttgtttaaatttattgagcaaaagggccagactactgatcatttctggcaaaacatgttgagtacattttagggtttaacagttttgggtacc

SEQ ID NO: 12 - DNA, 972 bp, Homo sapiens
gatcaggactcctcagttcaccttctcacaatgaggctccctgctcagctcctggggctgctaatgctctgggtctctggatccagtggggatattgtgatgactcagtctccactctccctgcccgtcacccctggagagccggcctccatctcctgcaggtctagtcagagcctcctgcatagtgatggatacaactatttggattggtacctgcagaagccagggcagtctccacagctcctgatctatttgggttctaatcgggcctccggggtccctgacaggttcagtggcagtggatcaggcacagattttacactgaaaatcagcaaagtggaggctgaggatgttgggatttattactgcatgcaaggtctacaaactcctcagacgttcggccaagggaccaaggtggaaatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacaccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttagagggagaagtgcccccacctgctcctcagttccagcctgaccccctcccatcctttggcctctgaccctttttccacaggggacctacccctattgcggtcctccagctcatctttcacctcacccccctcctcctccttggctttaattatgctaatgttggaggagaatgaataaataaagtgaatctttgaaaaaaaaaaaaaaaaaaaaaaaaaaaaa

SEQ ID NO: 13 - DNA, 835 bp, Homo sapiens
ggcacgaggctcaaccacagactacacttgctgaactggctcctggggccatgaggctgtcactgccactgctgctgctgctgctgggagcctgggccatcccagggggcctcggggacagggcgccactcacagccacagccccacaactggatgatgaggagatgtactcagcccacatgcccgctcacctgcgctgtgatgcctgcagagctgtggcttaccagatgtggcaaaatctggcaaaggcagagaccaaacttcatacctcaaactctggggggcggcgggagctgagcgagttggtctacacggatgtcctggaccggagctgctcccggaactggcaggactacggagttcgagaagtggaccaagtgaaacgtctcacaggcccaggacttagcgaggggccagagccaagcatcagcgtgatggtcacagggggcccctggcctaccaggctctccaggacatgtttgcactacttgggggagtttggagaagaccagatctatgaagcccaccaacaaggccgaggggctctggaggcattgctatgtgggggaccccagggggcctgctcagagaaggtgtcagccacaagagaagagctctagtcctggactctaccctcctctgaaagaagctggggcttgctctgacggtctccactcccgtctgcaggcagccaggagggcaggaagcccttgctctgtgctgccatcctgcctccctcctccagcctcagggcactcgggcctgggtgggagtcaacgccttcccctctggactcaaataaaacccagtgacctcaaaaaaaaaaaaaaaaaaa

SEQ ID NO: 14 - DNA, 1436 bp, Homo sapiens
gtccgcggaaatttgaaatggctgacgggtcgctgacgggcggcggtctggaggcagcggccatggcgccggagcgcacgggctgggcggtggagcaggagctggcgtctctggagaaagtttttcagaagaagtgaagtcaagatgaagaaccatttgcttttctggggagtcctggcggtttttattaaggctgttcatgtgaaagcccaagaagatgaaaggattgttcttgttgacaacaaatgtaagtgtgcccggattacttccaggatcatccgttcttccgaagatcctaatgaggacattgtggagagaaacatccgaattattgttcctctgaacaacagggagaatatctctgatcccacctcaccattgagaaccagatttgtgtaccatttgtctgacctctgtaaaaaatgtgatcctacagaagtggagctggataatcagatagttactgctacccagagcaatatctgtgatgaagacagtgctacagagacctgctacacttatgacagaaacaagtgctacacagctgtggtcccactcgtatatggtggtgagaccaaaatggtggaaacagccttaaccccagatgcctgctatcctgactaatttaagtcattgctgactgcatagctctttttcttgagaggctctccattttgattcagaaagttagcatatttattaccaatgaatttgaaaccagggcttttttttttttttgggtgatgtaaaaccaactccccgccaccaaaataattaaaatagtcacattgttatctttattaggtaatcacttcttaattatatgttcatactctaagtatcaaaatcttccaattatcatgctcacctgaaagaggtatgctctcttaggaatacagtttctagcattaaacaaataaacaaggggagaaaataaaactcaaggagtgaaaatcaggaggtgtaataaaatgttcctcgcattcccccccgcttttttttttttttgactttgccttggagagccagagcttccgcattttctttactattctttttaaaaaaagtttcactgtgtagagaacatatatgcataaacataggtcaattatatgtctccattagaaaaataataattggaaaacatgttctagaactagttacaaaaataatttaaggtgaaatctctaatatttataaaagtagcaaaataaatgcataattaaaatatatttggacataacagacttggaagcagatgatacagacttctttttttcataatcaggttagtgtaagaaattgccatttgaaacaatccattttgtaactgaaccttatgaaatatatgtatttcatggtacgtattctctagcacagtctgagcaattaaatagattcataagaaaaaaaaaaaaaaaaaaaaaaaaaaaaaa

SEQ ID NO: 15 - DNA, 660 bp, Homo sapiens
atgtccgagactgcgcctgccgcgcccgctgctccggcccctgccgagaagactcccgtgaagaagaaggcccgcaagtctgcaggtgcggccaagcgcaaagcgtctgggcccccggtgtccgagctcattactaaagctgttgccgcctccaaggagcgcagcggcgtatctttggccgctctcaagaaagcgctggcagccgctggctatgacgtggagaaaaacaacagccgcatcaagctgggtctcaagagcctggtgagcaagggcaccctggtgcagaccaagggcaccggcgcgtcgggttccttcaaactcaacaagaaggcggcctctggggaagccaagcctaaggctaaaaaggcaggcgcggccaaggccaagaagccagcaggagcggcgaagaagcccaagaaggcgacgggggcggccacccccaagaagagcgccaagaagaccccaaagaaggcgaagaagccggctgcagctgctggagccaaaaaagcgaaaagcccgaaaaaggcgaaagcagccaagccaaaaaaggcgcccaagagcccagcgaaggccaaagcagttaaacccaaggcggctaaaccaaagaccgccaagcccaaggcagccaagccaaagaaggcggcagccaagaaaaagtag

SEQ ID NO: 16 - DNA, 750 bp, Homo sapiens
agcttccctctcctcctcaccctcctcactcactgtgcagggtcctgggcccagtctgtgctgactcagccaccctcagcgtctgggacccccgggcagagggtcaccatctcttgttctggaagcagctccaacatcggaagtaatactgtaaactggtaccagcagctcccaggaacggcccccaaactcctcatctatcgtaataatcagcggccctcaggggtccctgaccgattctctggctccaagtctggcacctcagcctccctggccatcagtgggctccagtctgaggatgaggctgattattactgtgcagcatgggatgacagcctgaatggtgtggtattcggcggagggaccaagctgaccgtcctaggtcagcccaaggctgccccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttcataggttctcaaccctcaccccccaccacgggagactagagctgcaggatcc

SEQ ID NO: 17 - DNA, 597 bp, Homo sapiens
atgcccctaggtctcctgtggctgggcctagccctgttgggggctctgcatgcccaggcccaggactccacctcagacctgatcccagccccacctctgagcaaggtccctctgcagcagaacttccaggacaaccaattccaggggaagtggtatgtggtaggcctggcagggaatgcaattctcagagaagacaaagacccgcaaaagatgtatgccaccatctatgagctgaaagaagacaagagctacaatgtcacctccgtcctgtttaggaaaaagaagtgtgactactggatcaggacttttgttccaggttgccagcccggcgagttcacgctgggcaacattaagagttaccctggattaacgagttacctcgtccgagtggtgagcaccaactacaaccagcatgctatggtgttcttcaagaaagtttctcaaaacagggagtacttcaagatcaccctctacgggagaaccaaggagctgacttcggaactaaaggagaacttcatccgcttctccaaatatctgggcctccctgaaaaccacatcgtcttccctgtcccaatcgaccagtgtatcgacggctga

SEQ ID NO: 18 - DNA, 2112 bp, Homo sapiens
cgcgtcgctgcccagcccggtccggcgcgccacgcagtggatctctggacaggacaagactccgaagctactcccccagcacacagcccgggacccacaaacccagcttgcccccagccctcccacctgccactccctggcccctcccaccggccgccccccttggcgcgggcgcatggtgtgaaaggccaagtgctgaggcgggtatcatgggtgctgtgccctaggcctgggtggcagggggtgggtggcctgtgggtgtgccgggggggccagtgtgcccaccccagtctcttggcgtgctggagggcatcctggatggaattgaagtgaatggaacagaagccaagcaaggtggagtgtgggtcagacccagaggagaacagtgccaggtcaccagatggaaagcgaaaaagaaagaacggccaatgttccctgaaaagcagcatgtcagggtatatccctagttacctggacaaagacgagcagtgtgtcgtgtgtggggacaaggcaactggttatcactaccgctgtatcacttgtgagggctgcaagggcttctttcgccgcacaatccagaagaacctccatcccacctattcctgcaaatatgacagctgctgtgtcattgacaagatcacccgcaatcagtgccagctgtgccgcttcaagaagtgcatcgccgtggccatggccatggacttggttctagatgactcgaagcgggtggccaagcgtaagctgattgagcagaaccgggagcggcggcggaaggaggagatgatccgatcactgcagcagcgaccagagcccactcctgaagagtgggatctgatccacattgccacagaggcccatcgcagcaccaatgcccagggcagccattggaaacagaggcggaaattcctgcccgatgacattggccagtcacccattgtctccatgccggacggagacaaggtggacctggaagccttcagcgagtttaccaagatcatcaccccggccatcacccgtgtggtggactttgccaaaaaactgcccatgttctccgagctgccttgcgaagaccagatcatcctcctgaaggggtgctgcatggagatcatgtccctgcgggcggctgtccgctacgaccctgagagcgacaccctgacgctgagtggggagatggctgtcaagcgggagcagctcaagaatggcggcctgggcgtagtctccgacgccatcttcgaactgggcaagtcactctctgcctttaacctggatgacacggaagtggctctgctgcaggctgtgctgctaatgtcaacagaccgctcgggcctgctgtgtgtggacaagatcgagaagagtcaggaggcgtacctgctggcgttcgagcactacgtcaaccaccgcaaacacaacattccgcacttctggcccaagctgctgatgaaggagagagaagtgcagagttcgattctgtacaagggggcagcggcagaaggccggccgggcgggtcactgggcgtccacccggaaggacagcagcttctcggaatgcatgttgttcagggtccgcaggtccggcagcttgagcagcagcttggtgaagcgggaagtctccaagggccggttcttcagcaccagagcccgaagagcccgcagcagcgtctcctggagctgctccaccgaagcggaattctccatgcccgagcggtctgtggggaagacgacagcagtgaggcggactccccgagctcctctgaggaggaaccggaggtctgcgaggacctggcaggcaatgcagcctctccctgaagccccccagaaggccgatggggaaggagaaggagtgccataccttctcccaggcctctgccccaagagcaggaggtgcctgaaagctgggagcgtgggctcagcagggctggtcacctcccatcccgtaagaccaccttcccttcctcagcagccaaacatggccagactcccttgctttttgctgtgtagttccctctgcctgggatgcccttccccctttctctgcctggcaacatcttacttgtcctttgaggccccaactcaagtgtcacctccttccccagctcccccaggcagaaatag

SEQ ID NO: 19 - DNA, 975 bp, Homo sapiens
atgagccgcccgtcctccaccggccccagcgctaataaaccctgcagcaagcagccgccgccgcagccccagcacactccgtccccggctgcgcccccggccgccgccaccatctcggctgcgggccccggctcgtccgcggtgcccgccgcggcggcggtgatctcgggccccggcggcggcggcggggccggcccggtgtccccgcagcaccacgagctgacctcgctcttcgagtgtccggtctgctttgactatgtcctgcctcctattctgcagtgccaggccgggcacctggtgtgtaaccaatgccgccagaagttgagctgctgcccgacgtgcaggggcgccctgacgcccagcatcaggaacctggctatggagaaggtggcctcggcagtcctgtttccctgtaagtatgccaccacgggctgttccctgaccctgcaccatacggagaaaccagaacatgaagacatatgtgaataccgtccctactcctgcccatgtcctggtgcttcctgcaagtggcaggggtccctggaagctgtgatgtcccatctcatgcacgcccacaagagcattaccacccttcagggagaagacatcgtctttctagctacagacattaacttgccaggggctgtcgactgggtgatgatgcagtcatgttttggccatcacttcatgctggtgctggagaaacaagagaagtacgaaggccaccagcagttttttgccatcgtcctgctcattggcacccgcaagcaagccgagaactttgcctacagactggagttgaatgggaaccggcggagattgacctgggaggccacgccccgttcgattcatgacggtgtggctgcggccatcatgaacagcgactgccttgttttcgacacagccatagcacatctttttgcagataatgggaaccttggaatcaatgttactatttctacatgttgtccatga

SEQ ID NO: 20 - DNA, 650 bp, Homo sapiens
gtctcagtcaggacacagcatggacatgagggtccccgctcagctcctggggctcctgctacttcggctccgaggtgccagatgtgacatccagatgacccagtctccatcctccctgtctgcgtctgtaggagacagagtcaccatcacttgccgggcaagtcagagcattagcagctatttaaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaagttactactgtcaacagagttacaggacccccgcgtggacgttcggccaagggaccaaggtggaaatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgaga

SEQ ID NO: 21 - DNA, 851 bp, Homo sapiens
cccgcaagtgtacctcaatggcgagtttgtagggggctgtgacattcttctgcagatgcaccagaatggggacttggtggaagaactgaaaaagctggggatccactccgcccttttagatgaaaagaaagaccaagactccaagtgagggcggccaagtcctcgctgagcagagagggagccgttcatgtcagagactcactgccagaaaagccttacccattttggttttcactattgagaccgcaactgcttgcactgatcattttggttcatgagcagttggtgattttagttggtctggtgttcgggctaagaatattttattgtggacttaattacaaccactgcactgtaatgattcaatgctgtattatgatattgctgtaaacaaaattcattcttatattgtcacttattctttgcctgattcagaagttaaataggagctttggaatcattattcatgacccctctgcaaatgtgtcagtctccaaagagagtatctccccccaaattttgtgtagcttcttttgttatggaaaatggtggacaaaaaaagaaactgtgataactggggcgttgttttttaaaataaactccagcacagggatgctgtgcatgcctgagttgattccgaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 22 - DNA, 927 bp, Homo sapiens
ggaagtttaggttaactgtcttaaatttccaaagctgtaatcattattttcattctcaaagtgatggccttgtgttttgctcctctcctccagggccagactgagcccaggttgatttcaggcggacaccaatagactccacagcagctccaggagcccagacaccggcggccagaagcaaggctaggagctgctgcagccatgtcggccctcagcctcctcattctgggcctgctcacggcagtgccacctgccagctgtcagcaaggcctggggaaccttcagccctggatgcagggccttatcgcggtggccgtgttcctggtcctcgttgcaatcgcctttgcagtcaaccacttctggtgccaggaggagccggagcctgcacacatgatcctgaccgtcggaaacaaggcagatggagtcctggtgggaacagatggaaggtactcttcgatggcggccagtttcaggtccagtgagcatgagaatgcctatgagaatgtgcccgaggaggaaggcaaggtccgcagcaccccgatgtaaccttctctgtggctccaaccccaagactcccaggcacatgggatggatgtccagtgctaccacccaagccccctccttctttgtgtggaatctgcaatagtgggctgactccctccagccccatgccggccctacccgcccttgaagtatagccagccaaggttggagctcagaccgtgtctaggttggggctcggctgtggccctggggtctcctgctcagctcagaagagccttctggagaggacagtcagctgagcacctcccatcctgctcacacgtccttccccataactatggaaatggccctaatttctgtgaaataaagactttttgtatttctggggctgaggctcagcaacagcccctcaggcttccaaaa

SEQ ID NO: 23 - DNA, 897 bp, Homo sapiens
ctcgcttttcggttgccgttgtcttttttccttgactcggaaatgtccggtcgtggtaagcagggtggcaaggcgcgcgccaaggctaagtcgcgctcgtcgcgcgcggggctgcagttccccgtgggccgcgtgcaccggttgctccgcaagggcaactattcggagcgcgtgggcgccggcgccccggtctatctggccgcggtgctcgagtacttgactgccgagatcctggagcttgccggcaacgcggcgcgcgacaacaagaagacgcgcatcatcccgcgccacctgcagctggccatccgcaacgacgaggagctcaacaagctgctgggccgcgtgaccatcgcgcagggtggcgtcctgcccaacatccaggccgtactgctgcccaagaagacggagagccaccacaaggccaagggcaagtgaggccgcccgccgcccccggggcccctttgatggacataaaggctcttttcagagccacctaccatctcgagaaaagagccgcactgatcctgcagttctttataggccggaggcctgatcaccctaggctcatgaatgagcgcagtggccatggggaagggcgcaacgggaaccgagaccctggggactgattgggctgcatacttgcgaggtgggcaacgtgttctgttaacaacagggaaccctcgtccacaggtggccaccccttgctcttgagtcccacccaaaacctctagtagggttttaataacgctcaccgtaaaggtgtcttcataattactagtgacaagttctcttgactctagcaaggttcccgtgtggtcatcaagtacagaatgcaatttcttaatgatttatctgatattaaaagtatttatgatctctaaaaaaaaaaaaaaaaaa

SEQ ID NO: 24 - DNA, 2533 bp, Homo sapiens
ggagctcaagctcctctacaaagaggtggacagagaagacagcagagaccatgggacccccctcagcccctccctgcagattgcatgtcccctggaaggaggtcctgctcacagcctcacttctaaccttctggaacccacccaccactgccaagctcactattgaatccacgccattcaatgtcgcagaggggaaggaggttcttctactcgcccacaacctgccccagaatcgtattggttacagctggtacaaaggcgaaagagtggatggcaacagtctaattgtaggatatgtaataggaactcaacaagctaccccagggcccgcatacagtggtcgagagacaatataccccaatgcatccctgctgatccagaacgtcacccagaatgacacaggattctataccctacaagtcataaagtcagatcttgtgaatgaagaagcaaccggacagttccatgtatacccggagctgcccaagccctccatctccagcaacaactccaaccccgtggaggacaaggatgctgtggccttcacctgtgaacctgaggttcagaacacaacctacctgtggtgggtaaatggtcagagcctcccggtcagtcccaggctgcagctgtccaatggcaacatgaccctcactctactcagcgtcaaaaggaacgatgcaggatcctatgaatgtgaaatacagaacccagcgagtgccaaccgcagtgacccagtcaccctgaatgtcctctatggcccagatgtccccaccatttccccctcaaaggccaattaccgtccaggggaaaatctgaacctctcctgccacgcagcctctaacccacctgcacagtactcttggtttatcaatgggacgttccagcaatccacacaagagctctttatccccaacatcactgtgaataatagcggatcctatatgtgccaagcccataactcagccactggcctcaataggaccacagtcacgatgatcacagtctctggaagtgctcctgtcctctcagctgtggccaccgtcggcatcacgattggagtgctggccagggtggctctgatatagcagccctggtgtattttcgatatttcaggaagactggcagattggaccagaccctgaattcttctagctcctccaatcccattttatcccatggaaccactaaaaacaaggtctgctctgctcctgaagccctatatgctggagatggacaactcaatgaaaatttaaagggaaaaccctcaggcctgaggtgtgtgccactcagagacttcacctaactagagacagtcaaactgcaaaccatggtgagaaattgacgacttcacactatggacagcttttcccaagatgtcaaaacaagactcctcatcatgataaggctcttacccccttttaatttgtccttgcttatgcctgcctctttcgcttggcaggatgatgctgtcattagtatttcacaagaagtagcttcagagggtaacttaacagagtgtcagatctatcttgtcaatcccaacgttttacataaaataagagatcctttagtgcacccagtgactgacattagcagcatctttaacacagccgtgtgttcaaatgtacagtggtccttttcagagttggacttctagactcacctgttctcactccctgttttaattcaacccagccatgcaatgccaaataatagaattgctccctaccagctgaacagggaggagtctgtgcagtttctgacacttgttgttgaacatggctaaatacaatgggtatcgctgagactaagttgtagaaattaacaaatgtgctgcttggttaaaatggctacactcatctgactcattctttattctattttagttggtttgtatcttgcctaaggtgcgtagtccaactcttggtattaccctcctaatagtcatactagtagtcatactccctggtgtagtgtattctctaaaagctttaaatgtctgcatgcagccagccatcaaatagtgaatggtctctctttggctggaattacaaaactcagagaaatgtgtcatcaggagaacatcataacccatgaaggataaaagccccaaatggtggtaactgataatagcactaatgctttaagatttggtcacactctcacctaggtgagcgcattgagccagtggtgctaaatgctacatactccaactgaaatgttaaggaagaagatagatccaattaaaaaaaattaaaaccaatttaaaaaaaaaaaagaacacaggagattccagtctacttgagttagcataatacagaagtcccctctactttaacttttacaaaaaagtaacctgaactaatctgatgttaaccaatgtatttatttctgtggttctgtttccttgttccaatttgacaaaacccactgttcttgtattgtattgcccagggggagctatcactgtacttgtagagtggtgctgctttaattcataaatcacaaataaaagccaattagctctataact

SEQ ID NO: 25 - DNA, 1020 bp, Homo sapiens
gaggaactgctcagttaggacccagacggaaccatggaagccccagcgcagcttctcttcctcctgctactctggctcccagataccactggagaaatagtgatgacgcagtctccagccaccctgtctgtgtctccaggggaaagagccaccctctcctgcagggccagtcagagtgttaccagcaacttagcctggtaccagcagacacctgggcagtctcccaggctcgtcatctatggtgcatccagcagggccagtggtgtcccagccaggttcagtggcagtgggtctgggacagagttcactctcaccatcagcagcctgcagtctgaagattttgcagtttattactgtcagcagtataataagtggccgcacacttttggccaggggaccaagctggacatcaaacgaactgtggctgcaccatctgtcttcatcttcccgccatctgatgagcagttgaaatctggaactgcctctgttgtgtgcctgctgaataacttctatcccagggaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttagagggagaagtgcccccacctgctcctcagttccagcctgaccccctcccatcctttggcctctgaccctttttccacaggggacctacccctattgcggtcctccagctcatctttcacctcacccccctcctcctccttggctttaattatgctaatgttggaggagaatgaataaataaagtgaatctttgcaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 26 - DNA, 1020 bp, Homo sapiens; identical to SEQ ID NO: 25

SEQ ID NO: 27 - DNA, 564 bp, Homo sapiens
cgactttcccgatcgccaggcaggagtttctctcggtgactactatcgctgtcatgtctggtcgtggcaagcaaggaggcaaggcccgcgccaaggccaagtcgcgctcgtcccgcgctggccttcagttcccggtagggcgagtgcatcgcttgctgcgcaaaggcaactacgcggagcgagtgggggccggcgcgcccgtctacatggctgcggtcctcgagtatctgaccgccgagatcctggagctggcgggcaacgcggctcgggacaacaagaagacgcgcatcatccctcgtcacctccagctggccatccgcaacgacgaggaactgaacaagctgctgggcaaagtcaccatcgcccagggcggcgtcttgcctaacatccaggccgtactgctccctaagaagacggagagtcaccacaaggcaaagggcaagtgaggctgacgtccggcccaagtgggcccagcccggcccgcgtctcgaaggggcacctgtgaactcaaaaggctcttttcagagccacccacgttttcaaataaaagagttgttaatgctg

SEQ ID NO: 28 - DNA, 2470 bp, Homo sapiens
acgaggcctggccggggcgggcggcgcgggggcggcatgagggcccgcggcccggggggctgaggcgcccgccgcctgccgcgggggccgctcgcgtcctccatggaggccggagaggaaccgctgctgctggccgaactcaagcccgggcgcccccaccagtttgattggaagtccagctgtgaaacctggagcgtcgccttctccccagatggctcctggtttgcttggtctcaaggacactgcatcgtcaaactgatcccctggccgttggaggagcagttcatccctaaagggtttgaagccaaaagccgaagtagcaaaaatgagacgaaagggcggggcagcccaaaagagaagacgctggactgtggtcagattgtctgggggctggccttcagcccgtggccttccccacccagcaggaagctctgggcacgccaccacccccaagtgcccgatgtctcttgcctggttcttgctacgggactcaacgatgggcagatcaagatctgggaggtgcagacagggctcctgcttttgaatctttccggccaccaagatgtcgtgagagatctgagcttcacacccagtggcagtttgattttggtctccgcgtcacgggataagactcttcgcatctgggacctgaataaacacggtaaacagattcaagtgttatcgggccacctgcagtgggtttactgctgttccatctccccagactgcagcatgctgtgctctgcagctggagagaagtcggtctttctatggagcatgaggtcctacacgttaattcggaagctagagggccatcaaagcagtgttgtctcttgtgacttctcccccgactctgccctgcttgtcacggcttcttacgataccaatgtgattatgtgggacccctacaccggcgaaaggctgaggtcactccaccacacccaggttgaccccgccatggatgacagtgacgtccacattagctcactgagatctgtgtgcttctctccagaaggcttgtaccttgccacggtggcagatgacagactcctcaggatctgggccctggaactgaaaactcccattgcatttgctcctatgaccaatgggctttgctgcacattttttccacatggtggagtcattgccacagggacaagagatggccacgtccagttctggacagctcctagggtcctgtcctcactgaagcacttatgccggaaagcccttcgaagtttcctaacaacttaccaagtcctagcactgccaatccccaagaaaatgaaagagttcctcacatacaggactttttaagcaacaccacatcttgtgcttctttgtagcagggtaaatcgtcctgtcaaagggagttgctggaataatgggccaaacatctggtcttgcattgaaatagcatttctttgggattgtgaatagaatgtagcaaaaccagattccagtgtactagtcatggatctttctctccctggcatgtgaaagtcagtcttagaggaagagattccacttgcacggcaacagagccttacgttaaattttcagtccagttatgaacagcaagtgttgaactctttctgcttgttttgattcaaagtgcagttactgatgttgttttgattatgcaactaagtaggcctccagagcctctctagtggcagagcagctcacactccctccgctgggaacgatggcttctgcctagtacttatccttgtgtttctgatgcagtggtagcattggttcaagttctctcctgctgtggtcagagttgcttcgatgttggccaagtgcttttcttcttgggctcccttctgacctgcaggacagttttcctggagccatttggtatgaggtattaatttagcttaactaaattacaggggactcagaggccgtgctcctgaccgatccagacactattactggctttttttttttttttttaacaatggtgtgcatgtgcaggaaatgacaaatttgtatgtcagattatacaaggatgtattcttaaaccgcatgactattcagatggctactgagttatcagtggccatttattagcatcatatttatttgtattttctcaacagatgttaaggtacaactgtgtttttctcgattatctaaaaaccatagtacttaaattgaacagttgcaaagatgtcttaattgtgtaaagaattggtgtagtcatgactttagctgatactcttatgtacgagatctgtctctgctgtttaacttcattggattaatcagctggtttcaactctactgcgaaacaaaaatagctccttaaaagtactgttctccttcagtggcatgtagttatctaatcaagacacctcattcaaacaaaacctgccttaggaaaatttaatatattttaaattattttaaaagaaatacaacatcttattctttagctttcaaaaaaaaaaaaaaaaaa

SEQ ID NO: 29 - DNA, 2374 bp, Homo sapiens
gggcgatgagagcgggtactgcgaactgccgggcgatgctgtcgctgccgccgtgatacggagagcaacagttccccagcaacacccctccccgacacaggcacacaccccccgacaggcacgcacacccaccccacagtgcccggctcggctgcgcctcctctattggcccaggaagcccacccagccccgccacgcagagcccagaaggaaagaaagcctcatgcctgagccgaggggagcaccatggatctgacaaaaatgggcatgatccagctgcagaaccctagccaccccacggggctactgtgcaaggccaaccagatgcggctggccgggactttgtgcgatgtggtcatcatggtggacagccaggagttccacgcccaccggacggtgctggcctgcaccagcaagatgtttgagatcctcttccaccgcaatagtcaacactatactttggacttcctctcgccaaagaccttccagcagattctggagtatgcatatacagccacgctgcaagccaaggcggaggacctggatgacctgctgtatgcggccgagatcctggagatcgagtacctggaggaacagtgcctgaagatgctggagaccatccaggcctcagacgacaatgacacggaggccaccatggccgatggcggggccgaggaagaagaggaccgcaaggctcggtacctcaagaacatcttcatctcgaagcattccagcgaggagagtgggtatgccagtgtggctggacagagcctccctgggcccatggtggaccagagcccttcagtctccacttcatttggtctttcagccatgagtcccaccaaggctgcagtggacagtttgatgaccataggacagtctctcctgcagggaactcttcagccacctgcagggcccgaggagccaactctggctgggggtgggcggcaccctggggtggctgaggtgaagacggagatgatgcaggtggatgaggtgcccagccaggacagccctggggcagccgagtccagcatctcaggagggatgggggacaaggttgaggaaagaggcaaagaggggcctgggaccccgactcgaagcagcgtcatcaccagtgctagggagctacactatgggcgagaggagagtgccgagcaggtgccacccccagctgaggctggccaggcccccactggccgacctgagcacccagcacccccgcctgagaagcatctgggcatctactccgtgttgcccaaccacaaggctgacgctgtattgagcatgccgtcttccgtgacctctggcctccacgtgcagcctgccctggctgtctccatggacttcagcacctatggggggctgctgccccagggcttcatccagagggagctgttcagcaagctgggggagctggctgtgggcatgaagtcagagagccggaccatcggagagcagtgcagcgtgtgtggggtcgagcttcctgataacgaggctgtggagcagcacaggaagctgcacagtgggatgaagacgtacgggtgcgagctctgcgggaagcggttcctggatagtttgcggctgagaatgcacttactggctcattcagcgggtgccaaagcctttgtctgtgatcagtgcggtgcacagttttcgaaggaggatgccctggagacacacaggcagacccatactggcactgacatggccgtcttctgtctgctgtgtgggaagcgcttccaggcgcagagcgcactgcagcagcacatggaggtccacgcgggcgtgcgcagctacatctgcagtgagtgcaaccgcaccttccccagccacacggctctcaaacgccacctgcgctcacatacaggcgaccacccctacgagtgtgagttctgtggcagctgcttccgggatgagagcacactcaagagccacaaacgcatccacacgggtgagaaaccctacgagtgcaatggctgtggcaagaagttcagcctcaagcatcagctggagacgcactatagggtgcacacaggtgagaagccctttgagtgtaagctctgccaccagcgctcccgggactactcggccatgatcaagcacctgagaacgcacaacggcgcctcgccctaccagtgcaccatctgcacagagtactgccccagcctctcctccatgcagaagcacatgaagggccacaagcccgaggagatcccgcccgactggaggatagagaagacgtacctctacctgtgctatgtgtgaagggaggcccgcggcggtggagccgagcggggagccaggaaagaagagttggagtgagatgaaggaaggactatgacaaataaaaaaaaaaaaaaaaaaaaaaaaa

SEQ ID NO: 30 - DNA, 393 bp, Homo sapiens
atgtctggacgtggaaagcaaggcggcaaagctcgggcaaaagctaaaacgcgttcttccagggccggtcttcagtttccagttggccgtgtgcaccgcctcctccgcaaaggcaactactccgaacgagtcggggccggcgctccagtgtacctggcagcggtgctggaatatctgacggccgagatcttagagctagctggcaacgcggctcgcgacaataagaagacccgcatcatcccgcgccacctgcagctagccatccgcaacgacgaggagctaaataagcttctaggtcgcgtgaccatcgcgcagggcggtgtcctgcccaacatccaggccgtattgctgcctaagaagacggagagccaccataaggccaagggcaagtga

SEQ ID NO: 31 - DNA, 857 bp, Homo sapiens
caggaaagatgcagccactcctgcttctgctggcctttctcctacccactggggctgaggcaggggagatcatcggaggccgggagagcaggccccactcccgcccctacatggcgtatcttcagatccagagtccagcaggtcagagcagatgtggagggttcctggtgcgagaagactttgtgctgacagcagctcattgctggggaagcaatataaatgtcaccctgggcgcccacaatatccagagacgggaaaacacccagcaacacatcactgcgcgcagagccatccgccaccctcaatataatcagcggaccatccagaatgacatcatgttattgcagctgagcagaagagtcagacggaatcgaaacgtgaacccagtggctctgcctagagcccaggagggactgagacccgggacgctgtgcactgtggccggctggggcagggtcagcatgaggaggggaacagatacactccgagaggtgcagctgagagtgcagagggataggcagtgcctccgcatcttcggttcctacgacccccgaaggcagatttgtgtgggggaccggcgggaacggaaggctgccttcaagggggattccggaggccccctgctgtgtaacaatgtggcccacggcatcgtctcctatggaaagtcgtcaggggttcctccagaagtcttcaccagggtctcaagtttcctgccctggataaggacaacaatgagaagcttcaaactgctggatcagatggagacccccctgtgactgactcttcttctcggggacacaggccagctccacagtgttgccagagccttaataaacgtccacagagtataaataacc

SEQ ID NO: 32 - DNA, 3250 bp, Homo sapiens
ccaacttatttaaaacaaaacaattttgtaggtattattatacccatttcacagatgatgataaatgagaccaatagaagttaaataacttgccaaaggccacacagctggtgagtgatggagaacgaattaaaactcaagtgagcataattctaaaagccatcttctcgttagtgtttctcactatccaggtctgcctttgccttatttaactgaagttaagccatccttacctgtgatcacctagcctctcagtttggggggatcattacagcgggtttttaactcccaatgttctggtccagtttgctttacatgttcttatttatacattgtcaaggatgacctcaggacagtacagcaaggacacagtggcacttcacattttgttcccacgaaatgactggggcataatctcagatcatcttcctttagaatgtggaaacatcagcagaagaatattagtctttatacaagtcaaatccaaaatgacacatgtgaaaactaatagagctgactttcagccatgatagctttggcacacctcacatccctttgttcaacctctcttccctcaacggagagctgcattcctgggaatttctgttgtgcacttttcccacttgccctgctgtcatttaaaggtgaacattctagttttgctaagaaaaccctttccttcatttggaatgaacagcaattttattacttttgaccttaaaatgagtttgctgccttcaaatcttttcagcgccttcatcacgctctgcttcggggcgatcttcttcctgccagactcctccaagctgctcagcggggtcctgttccactccagccccgccttgcagccggccgccgaccacaagcccgggcccggggcgcgcgccgaggacgcggccgaggggcgagcccggcgccgcgaggagggggcacccggggacccggaggccgccctggaggacaacttggccaggatccgcgaaaaccacgagcgggctctcagggaagccaaggagaccctgcagaagctgcccgaggagatccaaagagacatcctactggagaagaagaaggtggcccaggaccagctgcgtgacaaggcgccgttcagaggcctgcccccggtggacttcgtgcccccaatcggggtggagagccgggagcccgccgacgccgccatccgcgagaaaagggcaaagatcaaagagatgatgaaacatgcttggaataattataaaggttatgcctggggattaaatgaactcaaacctatatcaaaaggaggccattcaagcagtttgtttggtaacatcaaaggagcaactatagtagatgccctggatacactttttattatggaaatgaaacatgaatttgaagaagcaaaatcatgggttgaagaaaatttagattttaatgtgaatgctgaaatttctgtctttgaagtaaatatacgctttgttggtggactactctcagcctactatctgtctggagaagagatttttcgaaagaaagcagtggaacttggggtaaaattgctacctgcatttcatactccctctggaataccttgggcattgctgaatatgaaaagtggtattggaaggaactggccctgggcctctggaggcagcagtattctggcagaatttggaaccctgcatttggagtttatgcacttgagccacttatcaggaaaccccatctttgctgaaaaggtaatgaatattcgaacagtactgaacaaactggaaaaaccacaaggcctttatcctaactatctgaatcccagtagtggacagtggggtcaacatcatgtatcagttggaggacttggagacagcttctatgagtatttgctgaaggcctggttaatgtctgacaagacagatctggaagctaagaagatgtattttgatgctgttcaggctatcgagactcatttgatccgcaagtctagcagcggactaacttatatcgcagagtggaaaaggggcctcctggagcacaagatgggccacctgacctgcttcgcggggggcatgttcgcactcggggctgatgcagctcccgaaggcatggcccaacactaccttgaactcggggctgaaattgcccgtacttgtcatgaatcatataatcgaacatttatgaaactgggaccagaagctttcagatttgatggtggtgttgaagccatcgctacaagacaaaatgaaaaatactacatcttacggccagaagttatggagacttacatgtatatgtggagactgactcatgatccaaagtacaggaaatgggcctgggaagccgtagaggccttggaaaaccattgcagagtgaatggaggctattcaggcctaagggatgtttaccttcttcatgagagttatgatgatgtgcagcagagtttcttcctggcagagacattgaaatatttgtacctaatattttctgacgacgatcttcttccactggagcattggatcttcaatagcgaggcacatcttctccctatcctccctaaagataaaaaggaagttgaaatcagagaggaataaaaaagacatttatattttattctgctccattcccttcactgtataccttaataattccttttctggtaatcaggcacatgatgaactttgattagtaggtctgtgattaagttcttaaattgttttgcagtcttttatgtttattatcataggtataggtggacctaaattccttatcatatctttattaattcagccagtgtatccaccagttttttgtttatgtttttaagtaacctattatctctggatttcatgaaggtgtaatatcgtttttgttaaactgaatagaattgtatagcgatgacctcttaattataatttgatttgactgcaaaactttttcctcctctaagaggagatgatgtctgctttaagctgtaatgttttgccatgttgcaaaaagccataataataagtataaaaaagctttttcctttacaatttcatgttaatctggtttgtctgtccaccagagacagatcttctgtgacagcctccttatgcaggtctatcattatttgatagaatgtcttctaaaatacttcactcacattgtaattcaaattagaaagtcattccaaaaggtcatgtcatgttgacctcatttcatcggaactgcagtatatttttgttggttaattatattagtgttttctattttgaaaaaaaaaaaaaaaaaa

SEQ ID NO: 33 - DNA, 381 bp, Homo sapiens
atgcctgagccagcgaaatccgctcccgccccgaagaagggctccaagaaggccgtgaccaaggcgcagaagaaggacagcaagaagcgcaagcgcagccgcaaggagagctactccgtatacgtgtacaaggtgctgaaacaggtccaccccgacaccggcatctcctctaaagccatggggatcatgaattcctttgtcaacgacatcttcgagcgcatcgccggcgaggcttcccgcctggcgcattacaacaagcgctcgaccatcacctccagggagatccagacggccgtgcgcctgctgcttcccggggagctggccaagcacgctgtgtcagagggcaccaaggccgttaccaagtacaccagctccaagtaa

SEQ ID NO: 34 - DNA, 1113 bp, Homo sapiens
ggggcgacgtttagcgactattgcgcctgcgccagcgccggctgcgagactggggccgtggctgctggtcccgggtgatgctaggcggctccctgggctccaggctgttgcggggtgtaggtgggagtcacggacggttcggggcccgaggtgtccgcgaaggtggcgcagccatggcggcaggggagagcatggctcagcggatggtctgggtggacctggagatgacaggattggacattgagaaggaccagattattgagatggcctgtctgataactgactctgatctcaacattttggctgaaggtcctaacctgattataaaacaaccagatgagttgctggacagcatgtcagattggtgtaaggagcatcacgggaggtctggccttaccaaggcagtgaaggagagtacaattacattgcagcaggcagagtatgaatttctgtcctttgtacgacagcagactcctccagggctctgtccacttgcaggaaattcagttcatgaagataagaagtttcttgacaaatacatgccccagttcatgaaacatcttcattatagaataattgatgtgagcactgttaaagaactgtgcagacgctggtatccagaagaatatgaatttgcaccaaagaaggctgcttctcatagggcacttgatgacattagtgaaagcatcaaagagcttcagttttaccgaaataacatcttcaagaaaaaaatagatgaaaagaagaggaaaattatagaaaatggggaaaatgagaagaccgtgagttgatgccagttatcatgctgccactacatcgttatctggaggcaacttctggtggtttttttttctcacgctgatggcttggcagagcaccttcggttaacttgcatctccagattgattactcaagcagacagcacacgaaatactatttttctcctaatatgctgtttccattatgacacagcagctcctttgtaagtaccaggtcatgtccatcccttggtacatatatgcatttgcttttaaaccatttcttttgtttaaataaataaataagtaaataaagctagttctattgaaatgcaaaaaaaaaaaaaaaaaaaaa

SEQ ID NO: 35 - DNA, 467 bp, Homo sapiens
attcttgttatttgagtgctctttcactctcctccgccatgcccgacccggctaaatctgctcctgcccccaaaaagggctccaagaaagccgtaaccaaggcccagaaaaaggacggcaagaagcgcaagcgcagccgcaaagagagttactctatctacgtgtacaaggtgctgaagcaagtccaccccgacaccggcatctcatcgaaggccatgggcatcatgaactccttcgtcaatgacatctttgagcgcatcgctggcgaggcttcccgcctggcgcattacaacaagcgctcgaccatcacctccagggagatccagacggccgtgcgcctgctgctgcccggggagctggccaagcacgccgtgtccgagggcacaaaggccgtcaccaagtacaccagctccaagtgagctctcgcagctgccagcaatccaaaggctcttttcagagccactcac

SEQ ID NO: 36 - DNA, 3272 bp, Homo sapiens
gggcactgctttaaaactgggaaggaggaagacgaggccagggagccggagggtcaccaaggtagatttccagcagcgctagtccagctgaacactttccagccttgtttttcagcagctttgaggaaaagtatagtgatccgtatgtgaaactttcattgtacgtagcggatgagaatagagaacttgctttggtccagacaaaaacaattaaaaagacactgaacccaaaatggaatgaagaattttatttcagggtaaacccatctaatcacagactcctatttgaagtatttgacgaaaatagactgacacgagacgacttcctgggccaggtggacgtgccccttagtcaccttccgacagaagatccaaccatggagcgaccctatacatttaaggactttctcctcagaccaagaagtcataagtctcgagttaagggatttttgcgattgaaaatggcctatatgccaaaaaatggaggtcaagatgaagaaaacagtgaccagagggatgacatggagcatggatgggaagttgttgactcaaatgactcggcttctcagcaccaagaggaacttcctcctcctcctctgcctcccgggtgggaagaaaaagtggacaatttaggccgaacttactatgtcaaccacaacaaccggaccactcagtggcacagaccaagcctgatggacgtgtcctcggagtcggacaataacatcagacagatcaaccaggaggcagcacaccggcgcttccgctcccgcaggcacatcagcgaagacttggagcccgagccctcggagggcggggatgtccccgagccttgggagaccatttcagaggaagtgaatatcgctggagactctctcggtctggctctgcccccaccaccggcctccccaggatctcggaccagccctcaggagctgtcagaggaactaagcagaaggcttcagatcactccagactccaatggggaacagttcagctctttgattcaaagagaaccctcctcaaggttgaggtcatgcagtgtcaccgacgcagttgcagaacagggccatctaccaccgcccagtgccccagctgggagagcgcgttcatcaactgtcacgggtggtgaggaaccaacgccatcagtggcctatgtacataccacgccgggtctgccttcaggctgggaagaaagaaaagatgctaaggggcgcacatactatgtcaatcataacaatcgaaccacaacttggactcgacctatcatgcagcttgcagaagatggtgcgtccggatcagccacaaacagtaacaaccatctaatcgagcctcagatccgccggcctcgtagcctcagctcgccaacagtaactttatctgccccgctggagggtgccaaggactcacccgtacgtcgggctgtgaaagacaccctttccaacccacagtccccacagccatcaccttacaactcccccaaaccacaacacaaagtcacacagagcttcttgccacccggctgggaaatgaggatagcgccaaacggccggcccttcttcattgatcataacacaaagactacaacctgggaagatccacgtttgaaatttccagtacatatgcggtcaaagacatctttaaaccccaatgaccttggcccccttcctcctggctgggaagaaagaattcacttggatggccgaacgttttatattgatcataatagcaaaattactcagtgggaagacccaagactgcagaacccagctattactggtccggctgtcccttactccagagaatttaagcagaaatatgactacttcaggaagaaattaaagaaacctgctgatatccccaataggtttgaaatgaaacttcacagaaataacatatttgaagagtcctatcggagaattatgtccgtgaaaagaccagatgtcctaaaagctagactgtggattgagtttgaatcagagaaaggtcttgactatgggggtgtggccagagaatggttcttcttactgtccaaagagatgttcaacccctactacggcctctttgagtactctgccacggacaactacacccttcagatcaaccctaattcaggcctctgtaatgaggatcatttgtcctacttcacttttattggaagagttgctggtctggccgtatttcatgggaagctcttagatggtttcttcattagaccattttacaagatgatgttgggaaagcagataaccctgaatgacatggaatctgtggatagtgaatattacaactctttgaaatggatcctggagaatgaccctactgagctggacctcatgttctgcatagacgaagaaaactttggacagacatatcaagtggatttgaagcccaatgggtcagaaataatggtcacaaatgaaaacaaaagggaatatatcgacttagtcatccagtggagatttgtgaacagggtccagaagcagatgaacgcattcttggagggattcacagaactacttcctattgatttgattaaaatttttgatgaaaatgagctggagttgctcatgtgcggcctcggtgatgtggatgtgaatgactggagacagcattctatttacaagaacggctactgcccaaaccaccccgtcattcagtggttctggaaggctgtgctactcatggacgccgaaaagcgtatccggttactgcagtttgtcacagggacatcgcgagtacctatgaatggatttgccgaactttatggttccaatggtcctcagctgtttacaatagagcaatggggcagtcctgagaaactgcccagagctcacacatgctttaatcgccttgacttacctccatatgaaacctttgaagatttacgagagaaacttctcatggccgtggaaaatgctcaaggatttgaaggggtggattaagcaccctgtacctcgggggtggttgttcttcaagcaagttctgcttgcacttttgcatttgcctaacagacttttgcagaggcgatggcagagagcagctgcaggcatggtccctggagccgagccttcaccacgcactcgtccaagttcggatgcgggaacctggtcccagcttgagttcctgcctttcccaccacaaattatcaactggttgatgtgtacactaattacatttcaggaggacttaatgctatttatgttgtgcctctgcaggcaaagcccttaataaatattttacatccttaaaaaaaaaaaaaaaaaaaaaaaaaa

SEQ ID NO: 37 - DNA, 3215 bp, Homo sapiens
gacaatatcaggtgagctgtggaggtggggtccttggaagctggatgacagcagctggcaaggggataagagagcagtgagcccctccctcaaggaggtctggctttatccatagacagggccctctgaggtggggctgaggtacaaagggggattgagcagcccaggagaagagagatgggggttcccttcttctcttctctcagatgcatggtggacttaggaccttgctgggctgggggtctcactgcagagatgaagctgcttctggccctagcagggctcctggccattctggccacgccccagccctctgaaggtgctgctccagctgtcctgggggaggtggacacctcgttggtgctgagctccatggaggaggccaagcagctggtggacaaggcctacaaggagcggcgggaaagcatcaagcagcggcttcgcagcggctcagccagccccatggaactcctatcctacttcaagcagccggtggcagccaccaggacggcggtgagggccgctgactacctgcacgtggctctagacctgctggagaggaagctgcggtccctgtggcgaaggccattcaatgtcactgatgtgctgacgcccgcccagctgaatgtgttgtccaagtcaagcggctgcgcctaccaggacgtgggggtgacttgcccggagcaggacaaataccgcaccatcaccgggatgtgcaacaacagacgcagccccacgctgggggcctccaaccgtgcctttgtgcgctggctgccggcggagtatgaggacggcttctctcttccctacggctggacgcccggggtcaagcgcaacggcttcccggtggctctggctcgcgcggtctccaacgagatcgtgcgcttccccactgatcagctgactccggaccaggagcgctcactcatgttcatgcaatggggccagctgttggaccacgacctcgacttcacccctgagccggccgcccgggcctccttcgtcactggcgtcaactgcgagaccagctgcgttcagcagccgccctgcttcccgctcaagatcccgcccaatgacccccgcatcaagaaccaagccgactgcatcccgttcttccgctcctgcccggcttgccccgggagcaacatcaccatccgcaaccagatcaacgcgctcacttccttcgtggacgccagcatggtgtacggcagcgaggagcccctggccaggaacctgcgcaacatgtccaaccagctggggctgctggccgtcaaccagcgcttccaagacaacggccgggccctgctgccctttgacaacctgcacgatgacccctgtctcctcaccaaccgctcagcgcgcatcccctgcttcctggcaggggacacccgttccagtgagatgcccgagctcacctccatgcacaccctcttacttcgggagcacaaccggctggccacagagctcaagagcctgaaccctaggtgggatggggagaggctctaccaggaagcccggaagatcgtgggggccatggtccagatcatcacttaccgggactacctgcccctggtgctggggccaacggccatgaggaagtacctgcccacgtaccgttcctacaatgactcagtggacccacgcatcgccaacgtcttcaccaatgccttccgctacggccacaccctcatccaacccttcatgttccgcctggacaatcggtaccagcccatggaacccaacccccgtgtccccctcagcagggtcttttttgcctcctggagggtcgtgctggaaggtggcattgaccccatcctccggggcctcatggccacccctgccaagctgaatcgtcagaaccaaattgcagtggatgagatccgggagcgattgtttgagcaggtcatgaggattgggctggacctgcctgctctgaacatgcagcgcagcagggaccacggcctcccaggatacaatgcctggaggcgcttctgtgggctcccgcagcctgaaactgtgggccagctgggcacggtgctgaggaacctgaaattggcgaggaaactgatggagcagtatggcacgcccaacaacatcgacatctggatgggcggcgtgtccgagcctctgaagcgcaaaggccgcgtgggcccactcctcgcctgcatcatcggtacccagttcaggaagctccgggatggtgatcggttttggtgggagaacgagggtgtgttcagcatgcagcagcgacaggccctggcccagatctcattgccccggatcatctgcgacaacacaggcatcaccaccgtgtctaagaacaacatcttcatgtccaactcatatccccgggactttgtcaactgcagtacacttcctgcattgaacctggcttcctggagggaagcctcctagaggccaggtaagggggtgcagcagtgaggggtatatctgggctggccagttggaaccacggagatctccttgccctagatgagcccagccctgttctgggtgcagctgagaaaatgagtgactagacgttcatttgtgtgctcatgtatgtgcgaagtatataaattggcttttcatgcgtgtgtgttgtctgaacatggggagtgtttcatgggttatgtgtatgtgccatttatgtgagtgtgtgtttgtgctgatgagaatactgagtatgtggaaggcagcagagcggactggtgaggagcacagctcaggaactagactgcctgggttccaatcctggctctgtggcttgctagctatgtgaccttgagcaaattaccctccttaaacaagagttttcttccttgtaaattacatctgtcatggtttcttggagggcccacttgtatcctctggttcttcatttattgagcacctactacatgcaaggcactgtactaggcgtgagaagcatatagaggcaagaaagagataccaagatgccatctgtgtcctggttagcagagctggaccagtggtgccttggagggataagccagctgcagctgggctgtgtggttgacttatgggcccagccagccaggctcaggccatggctcccctttttcttcctcaccctgatttcttgcttattcactgaagttctcctgaagaggaactgggcctgttgccctttctgtaccatttatttgctcccaatgtttatgataataaaggcaccgctgatggggacctcc

SEQ ID NO: 38 - DNA, 726 bp, Homo sapiens
gccttccttcctgcttcgcctccgcgcctcgcgctatgggacagagcccccgatccgccagcaccacctgaggatccagaaaccgccccagcgatggaagaggatcaggagctggagagaaaaatatctggattgaagacctcaatggctgaaggcgagaggaagacagccctggaaatggtccaggcagctggaacagatagacactgtgtgacatttgtattgcacgaggaagaccataccctaggaaattctctacgttacatgatcatgaagaacccggaagtggaattttgtggttacactacgacccatccttcagagagcaaaattaatttacgcattcagactcgaggtacccttccagctgttgagccatttcagagaggcctgaatgagctcatgaatgtctgccaacatgtgcttgacaagtttgaggccagcataaaggactataaggatcaaaaagcaagcagaaatgaatccacattctagtcctttatgcagtatacaaggagaactgtcctgtaggatattctcttcctgatggtgcagaacccagaattagaagtttgtggttacagcatactctgtccttcagaaaggcgtgattctagctgttgaccccttgcagctgttggaatctctgcaagaacctctgtattcttctaataaattccctcttttatttaaaaaaaaaaaaaaaaaaaaaaaaaaaaaaaaaaa

SEQ ID NO: 39 - DNA, 381 bp, Homo sapiens
atgcctgaacctaccaagtctgctcctgccccaaagaagggctccaagaaggcggtgactaaggctcagaagaaggacgggaagaagcgcaagcgcagccgcaaggagagctattcagtgtatgtgtacaaggtgctgaagcaggtccatcccgacaccggcatctcttccaaggcaatggggatcatgaattccttcgtcaacgacatcttcgagcgcatcgcaggcgaggcttcccgcctggcgcattacaacaagcgctcgaccatcacctccagggagatccagacggccgtgcgcctgctgcttccgggggagctggccaagcacgccgtgtcggagggcaccaaggccgtcaccaagtacaccagttccaagtaa

SEQ ID NO: 40 - DNA, 1922 bp, Homo sapiens
agacacgtggtccgggtggaagtgtccctgctgcgagcaggagctcacgctgggagggcagacacatggtcccgtggaagtgtccctgctgcaagcaggagcgctagtgctgggagggcggacacgtggctccgggcagaagtgtccgccagcaggagcgctcgtgcttggaaggtagacacgtggcccgggcggaagtatccttgcagcgagcaggagctggcgctgggagggcagacacgtggtccgggcggaagtgtctgtgcagccagcgggagctcgcgctgggagcggagacaggccctgccctgggagaagccctgccacacgtcgtgcccacgctgagggcctgtctgcagccctcccaagacccgcagatgcgcctgaagctgttctccatcctgtccaccgtgctgctcagagccacggacaccatcaactcccaggggcagtttcccagctacctcgagacggtgacaaaggacatcctggcccccaatctgcagtggcatgcggggaggacagccgcggccatccgcacggctgccgtgtcctgcctctgggcgctcaccagcagcgaggtcctgtcggcagagcagatacgggacgtgcaggaaacactgatgccccaggtcctgaccaccctggaggaggattcgaagatgacgcgactgatctcatgccgtattatcaacacgttcttaaaaacctcgggcggcatgacggatccagagaaactcatcaagatttatcctgaactcttaaaacgcctagatgacgtgtccaacgatgtgaggatggcagccgcctccaccttggtcacctggctgcagtgtgtcaagggtgccaacgcaaaatcctactatcagagcagtgtccagtacctgtaccgagagttgctggttcaccttgacgatccagagagggccatccaggatgcaattttagaggtcctcaaagagggcagcgggctgttcccagatctcctggtgagggagacggaggccgtcatccacaagcaccgctcggccacctactgcgagcagctcctgcagcatgtgcaggccgtgccagccacacagtgaccacgctggtttcagccacggcacacccttgtccccacctgagccagagtttgtggcctttaaatctcataaacaaggcacctctgtgccagcagtgagactgtgacagcaagaatgtactcctcaggacacctgcccgctctttccctggaataacagcctctgagtggattctgcatgttatgtgatttgttctgttcatcaagagggctcccaaacatctgcagctgatttgaaattaaaagtaagtcgcagccgctcctcccgcagccacttcagcagcatcttagattttaagcctcacgtgcgcagctggttcatgaactattggctgcatcctgcttaggtgcccaccaagaaggtttttacctacttaacaaaaaagaaagaagccaaagtgattagaaagaaatgaaatctctttttgggttctgtctactgaaatttaatatctcagtgaacagactaaaaggaatttagaatcctaacaacttaccagatttctcctgttttaaatatactgggactttaaaggttatatgtccggtcaccgtatgttttaagtcggtgttaatgctaacagtgttgaaaacaatatttcatgagatctaattgtggttgcccctataggtagcaggaaagtaaagttgcatttccctctcgcacattctacacccaagtgcctaaaagatctcattgtaagtgggtagtgttaccggaagccattgtgttcacacgggggaaatgccgtatatatttttcaacaaatattaacgtttatactttcatgtttgaaaatttaattaaaaatatttgttttaaaaaaaaaaaaaaaaa

SEQ ID NO: 41 - DNA, 1421 bp, Homo sapiens
acttactgcgggacggccttggagagtactcgggttcgtgaacttcccggaggcgcaatgagctgcattaacctgcccactgtgctgcccggctcccccagcaagacccgggggcagatccaggtgattctcgggccgatgttctcaggaaaaagcacagagttgatgagacgcgtccgtcgcttccagattgctcagtacaagtgcctggtgatcaagtatgccaaagacactcgctacagcagcagcttctgcacacatgaccggaacaccatggaggcgctgcccgcctgcctgctccgagacgtggcccaggaggccctgggcgtggctgtcataggcatcgacgaggggcagtttttccctgacatcatggagttctgcgaggccatggccaacgccgggaagaccgtaattgtggctgcactggatgggaccttccagaggaagccatttggggccatcctgaacctggtgccgctggccgagagcgtggtgaagctgacggcggtgtgcatggagtgcttccgggaagccgcctataccaagaggctcggcacagagaaggaggtcgaggtgattgggggagcagacaagtaccactccgtgtgtcggctctgctacttcaagaaggcctcaggccagcctgccgggccggacaacaaagagaactgcccagtgccaggaaagccaggggaagccgtggctgccaggaagctctttgccccacagcagattctgcaatgcagccctgccaactgagggacctgcaagggccgcccgctcccttcctgccactgccgcctactggacgctgccctgcatgctgcccagccactccaggaggaagtcgggaggcgtggagggtgaccacaccttggccttctgggaactctcctttgtgtggctgccccacctgccgcatgctccctcctctcctacccactggtctgcttaaagcttccctctcagctgctgggacgatcgcccaggctggagctggccccgcttggtggcctgggatctggcacactccctctccttggggtgagggacagagccccacgctgttgacatcagcctgcttcttcccctctgcggctttcactgctgagtttctgttctccctgggaagcctgtgccagcacctttgagccttggcccacactgaggcttaggcctctctgcctgggatgggctcccaccctcccctgaggatggcctggattcacgccctcttgtttccttttgggctcaaagcccttcctacctctggtgatggtttccacaggaacaacagcatctttcaccaagatgggtggcaccaaccttgctgggacttggatcccaggggcttatctcttcaagtgtggagagggcagggtccacgcctctgctgtagcttatgaaattaactaatt

SEQ ID NO: 42 - DNA, 999 bp, Homo sapiens
ggcacgaggggcgcaagccggcaagatggcggcggctggggctggccgtctgaggcgggtggcatcggctctgctgctgcggagcccccgcctgcccgcccgggagctgtcggccccggcccgactctatcacaagaaggttgttgatcattatgaaaatcctagaaacgtggggtcccttgacaagacatctaaaaatgttggaactggactggtgggggctccagcatgtggtgacgtaatgaaattacagattcaagtggatgaaaaggggaagattgtggatgctaggtttaaaacatttggctgtggttccgcaattgcctccagctcattagccactgaatgggtgaaaggaaagacggtggaggaagccttgactatcaaaaacacagatatcgccaaggagctctgccttcctcccgtgaaactgcactgctccatgctggctgaagatgcaatcaaggccgccctggctgattacaaattgaaacaagaacccaaaaaaggagaggcagagaagaaatgagccctccctcggcgaagcctccagcaggccacaccagctgtttcccacctgctgtgcagtcaccttagatgttcagaagccgcttcctctccactgaagagctatgagatacgcacaatacttgctgttcacgttatgactctcatgcaagcaaaatacacagtttcattgttctgaatcctgtggtttctttcagcccacttttatcgccttaacctagttaatgtatattttgaattgtgtgtatgacctcagaactgaaattgataatgaagttgcaagttttgatagcccgtgaagtgcataagtatctaattttacctgaattgatttggggggaaattaccagtagaatgccttggtctgaatatttgatagaaccaattgttgtacataaaacagatctgcgcatatatatatatgtataaaaaataataaaataatggaagatgaaaaaaaaaaaaaaaaaa

SEQ ID NO: 43 - DNA, 487 bp, Homo sapiens
actcactttctgacttaggccacaggtcgttttaccatgtctggacgtggcaagcagggcggcaaggctcgcgccaaggccaaaacccgctcctctagagctgggctccaatttcctgtaggacgagtgcaccgcctgctccgcaagggcaactacgctgagcgggtcggggccggcgcgccggtttacctggcggcggtgctggagtacctaactgccgagatcctggagctggcgggcaacgcagcccgcgacaacaaaaagacccgcatcatcccgcgccacttgcagctggccatccgcaacgacgaggagctcaacaagctgcttggtaaagttaccatcgctcagggcggtgttctgcctaacatccaggccgtactgctccccaagaagactgagagccaccacaaagctaagggcaagtaagggctgaactttaaaaatgtaaacttacaagacaaaaggctcttttcagagccaccca

SEQ ID NO: 44 - DNA, 833 bp, Homo sapiens
ggccacccgcctttcactatccgccattcttgtcacctcagctgctgccctcgctaccgcaccgacttcgcccgtgtgctcgcctgcacttgcgctgcccgccatggccaccgcccagccgtcgcaggtgcgccagaagtacgacaccaactgcgacgccgccatcaacagccacatcacgctggagctctacacctcctacctgtacctgtctatggccttctacttcaaccgggacgacgtggccctggagaacttcttccgctacttcctgcgcctgtcggacgacaaaatggagcatgcccagaagctgatgaggctgcagaacctgcgcggtggccacatctgccttcacgatatcaggaagccagagtgccaaggctgggagagcgggctcgtggccatggagtccgccttccacctggagaagaacgtcaaccagagcctgctggatctgtaccagctggccgtggagaagggcgacccccagctgtgccacttcctggagagccactacctgcacgagcaagtcaagaccatcaaagagctgggtggctacgtgagcaacctgcgcaagatttgttccccggaagccggcctggctgagtacctgttcgacaagctcaccctgggcggccgcgtcaaagagacttgagcccagatgggccccacagccacggggtcccttccctgggtcaggccactaggcggggcgtgcatgttgccctttcagaacgttctcttcagttttatctttcagttttaccattgttagcaaaaaagttatctggttctcaaagcaataaaggtgtccataaaaaaaaaaaaaaaaa

SEQ ID NO: 45 - DNA, 7149 bp, Homo sapiens
atgtctggcggcgccgcagagaagcagagcagcactcccggttccctgttcctctcgccgccggctcctgcccccaagaatggctccagctccgattcctccgtgggggagaaactgggagccgcggccgccgacgctgtgaccggcaggaccgaggagtacaggcgccgccgccacactatggacaaggacagccgtggggcggccgcgaccactaccaccactgagcaccgcttcttccgccggagcgtcatctgcgactccaatgccactgcgctggagcttcccggccttcctctttccctgccccagcccagcatccccgcggctgtcccgcagagtgctccaccggagccccaccgggaagagaccgtgaccgccaccgccacttcccaggtagcccagcagcctccagccgctgccgcccctggggaacaggccgtcgcgggccctgccccctcgactgtccccagcagtaccagcaaagaccgcccagtgtcccagcctagccttgtggggagcaaagaggagccgccgccggcgagaagtggcagcggcggcggcagcgccaaggagccacaggaggaacggagccagcagcaggatgatatcgaagagctggagaccaaggccgtgggaatgtctaacgatggccgctttctcaagtttgacatcgaaatcggcagaggctcctttaagacggtctacaaaggtctggacactgaaaccaccgtggaagtcgcctggtgtgaactgcaggatcgaaaattaacaaagtctgagaggcagagatttaaagaagaagctgaaatgttaaaaggtcttcagcatcccaatattgttagattttatgattcctgggaatccacagtaaaaggaaagaagtgcattgttttggtgactgaacttatgacgtctggaacacttaaaacgtatctgaaaaggtttaaagtgatgaagatcaaagttctaagaagctggtgccgtcagatccttaaaggtcttcagtttcttcatactcgaactccacctatcattcaccgcgatcttaaatgtgacaacatctttatcaccggccctactggctcagtcaagattggagacctcggtctggcaaccctgaagcgggcttcttttgccaagagtgtgataggtaccccagagttcatggcccctgagatgtatgaggagaaatatgatgaatccgttgacgtttatgcctttgggatgtgcatgcttgagatggctacatctgaatatccttactcggagtgccaaaatgctgcgcagatctaccgtcgcgtgaccagtggggtgaagccagccagttttgacaaagtagcaattcctgaagtgaaggaaattattgaaggatgcatacgacaaaacaaagatgaaagatattccatcaaagaccttttgaaccatgccttcttccaagaggaaacaggagtacgggtagaattagcagaggaagatgatggagaaaaaatagccataaaattatggctacgtattgaagatattaagaaattaaagggaaaatacaaagataatgaagctattgagttttcttttgatttagagagagatgtcccagaagatgttgcacaagaaatggtagagtctgggtatgtctgtgaaggtgatcacaagaccatggctaaagctatcaaagacagagtatcattaattaagaggaaacgagagcagcggcagttggtacgggaggagcaagaaaaaaaaaagcaggaagagagcagtctcaaacagcaggtagaacaatccagtgcttcccagacaggaatcaagcagctcccttctgctagcaccggcatacctactgcttctaccacttcagcttcagtttctacacaagtagaacctgaagaacctgaggcagatcaacatcaacaactacagtaccagcaacccagtatatctgtgttatctgatgggacggttgacagtggtcagggatcctctgtcttcacagaatctcgagtgagcagccaacagacagtttcatatggttcccaacatgaacaggcacattctacaggcacagtcccagggcatataccttctactgtccaagcacagtctcagccccatggggtatatccaccctcaagtgtggcacaggggcagagccagggtcagccatcctcaagtagcttaacaggggtttcatcttcccaacccatacaacatcctcagcagcagcagggaatacagcagacagcccctcctcaacagacagtgcagtattcactttcacagacatcaacctccagtgaggccactactgcacagccagtgagtcagcctcaagctccacaagtcttgcctcaagtatcagctggaaaacagcttccagtttcccagccagtaccaactatccaaggcgaacctcagatcccagttgcgacacaaccctcggttgttccagtccactctggtgctcatttccttccagtgggacagccgctccctactcccttgctccctcagtaccctgtctctcagattcccatatcaactcctcatgtgtctacggctcagacaggtttctcatcccttcccatcacaatggcagctggcattactcagcctctgctcacgttggcttcatctgctacaacagctgcgatcccgggggtatcaactgtggttcctagtcagcttccaacccttctgcagcctgtgactcagctgccaagtcaggttcacccacagctcctacaaccagcagttcagtccatgggaataccagctaaccttggacaagctgctgaggttccactttcctctggagatgttctgtaccagggcttcccacctcgactgccaccacagtacccaggagattcaaatattgctccctcttccaacgtggcttctgtttgcatccattctacagtcctatcccctcccatgccgacagaagtactggctacacctgggtactttcccacagtggtgcagccttatgtggaatcaaatcttttagttcctatgggtggtgtaggaggacaggttcaagtgtcccagccaggagggagtttagcacaagcccccactacatcctcccagcaagcagttttggagagtactcagggagtctctcaggttgctcctgcagagccagttgcagtagcacagccccaagctacccagccgaccactttggcttcctctgtagacagtgcacattcagatgttgcttcaggtatgagtgatggcaatgagaacgtcccatcttccagtggaaggcatgaaggaagaactacaaaacggcattaccgaaaatctgtaaggagtcgctctcgacatgaaaaaacttcacgcccaaaattaagaattttgaatgtttcaaataaaggagaccgagtagtagaatgtcaattagagactcataataggaaaatggttacattcaaatttgacctagatggtgacaaccccgaggagatagcaacaattatggtgaacaatgactttattctagcaatagagagagagtcgtttgtggatcaagtgcgagaaattattgaaaaagctgatgaaatgctcagtgaggatgtcagtgtggaaccagagggtgatcagggattggagagtctacaaggaaaggatgactatggcttttcaggttctcagaaattggaaggagagttcaaacaaccaattcctgcgtcttccatgccacagcaaataggcattcctaccagttctttaactcaagttgttcattctgcgggaaggcggtttatagtgagtcctgtgccagaaagccgattacgagaatcaaaagttttccccagtgaaataacagatacagttgctgcctctacagctcagagccctggaatgaacttgtctcactctgcatcatcccttagtctacaacaggccttttctgaacttagacgtgcccaaatgacagaaggacccaacacagcacctccaaactttagtcatacaggaccaacatttccagtagtacctcctttcttaagtagcattgctggagtcccaaccacagcagcagccacagcaccagtccctgcaacaagcagccctcctaatgacatttccacatcagtaattcagtctgaggttacagtgcccactgaagaggggattgctggagttgccaccagcacaggtgtggtaacttcaggtggtctccccataccacctgtgtctgaatcaccagtactttccagcgtagtttcaagtatcacaatacctgcagttgtctcaatatctactacatccccgtcacttcaagtccccacatccacatctgagatcgttgtttctagtacagcactgtatccttcagtaacagtttcagcaacttcagcctctgcagggggcagtactgctaccccaggtcctaagcctccagctgtagtatctcagcaggcagcaggcagcactactgtgggagccacattaacatcagtttctaccaccacttcattcccaagcacagcttcacagctgtccattcagcttagcagcagtacttctactcctactttagctgaaaccgtggtagttagcgcacactcactagataagacatctcatagcagtacaactggattggctttctccctctctgcaccatcttcctcttcctctcctggagcaggagtgtctagttatatttctcagcctggtgggctgcatcctttggtcattccatcagtgatagcttctactcctattcttccccaagcagcaggacctacttctacacctttattaccccaagtacctagtatcccacccttggtacagcctgttgccaatgtgcctgctgtacagcagacactaattcatagtcagcctcaaccagctttgcttcccaaccagccccatactcattgtcctgaagtagattctgatacacaacccaaagctcctggaattgatgacataaagactctagaagaaaagctgcggtctctgttcagtgaacacagctcatctggagctcagcatgcctctgtctcactggagacctcactagtcatagagagcactgtcacaccaggcatcccaactactgctgttgcaccaagcaaactcctgacttctaccacaagtacttgcttaccaccaaccaatttaccactaggaacagttgctttgccagttacaccagtggtcacacctgggcaagtttctaccccagtcagcactactacatcaggagtgaaacctggaactgctccctccaagccacctctaactaaggctccggtgctgccagtgggtactgaacttccagcaggtactctacccagcgagcagctgccaccttttccaggaccttctctaacccagtcccagcaacctctagaggatcttgatgctcaattgagaagaacacttagtccagagattatcacagtgacttctgcggttggtcctgtgtccatggcggctccaacagcaatcacagaagcaggaacacagcctcagaagggtgtttctcaagtcaaagaaggccctgtcctagcaactagttcaggagctggtgtttttaagatgggacgatttcaggtttctgttgcagcagacggtgcccagaaagagggtaaaaataagtcagaagatgcaaagtctgttcattttgaatccagcacctcagagtcctcagtgctatcaagtagtagtccagagagtaccttggtgaaaccagagccgaatggcataaccatccctggtatctcttcagatgtgccagagagtgcccacaaaactactgcctcagaggcaaagtcagacactgggcagcctaccaaggttggacgttttcaggtgacaactacagcaaacaaagtgggtcgtttctctgtatcaaaaactgaggacaagatcactgacacaaagaaagaaggaccagtggcatctcctccttttatggatttggaacaagctgttcttcctgctgtgataccaaagaaagagaagcctgaactgtcagagccttcacatctaaatgggccgtcttctgacccggaggccgcttttttaagtagggatgtggatgatggttccggtagtccacactcgccccatcagctgagctcaaagagccttcctagccagaatctaagtcaaagccttagtaattcatttaactcctcttacatgagtagcgacaatgagtcagatatcgaagatgaagacttaaagttagagctgcgacgactacgagataaacatctcaaagagattcaggacctgcagagtcgccagaagcatgaaattgaatctttgtataccaaactgggcaaggtgccccctgctgttattattcccccagctgctcccctttcagggagaagacgacgacccactaaaagcaaaggcagcaaatctagtcgaagcagttccttggggaataaaagcccccagctttcaggtaacctgtctggtcagagtgcagcttcagtcttgcacccccagcagaccctccaccctcctggcaacatcccagagtccgggcagaatcagctgttacagccccttaagccatctccctccagtgacaacctctattcagccttcaccagtgatggtgccatttcagtaccaagcctttctgctccaggtcaaggaaccagcagcacaaacactgttggggcaacagtgaacagccaagccgcccaagctcagcctcctgccatgacgtccagcaggaagggcacattcacagatgacttgcacaagttggtagacaattgggcccgagatgccatgaatctctcaggcaggagaggaagcaaagggcacatgaattacgagggccctggaatggcaaggaagttctctgcacctgggcaactgtgcatctccatgacctcgaacctgggtggctctgcccccatctctgcagcatcagctacctctctaggtcacttcaccaagtctatgtgccccccacagcagtatggctttccagctaccccatttggcgctcaatggagtgggacgggtggcccagcaccacagccacttggccagttccaacctgtgggaactgcctccttgcagaatttcaacatcagcaatttgcagaaatccatcagcaaccccccaggctccaacctgcggaccacttag

SEQ ID NO: 46 - DNA, 2168 bp, Homo sapiens
ggcgcgcgtgaacgcggtccccgggaccatgctgcggccacagcggcccggagacttgcagctcggggcctccctctacgagctggtgggctacaggcagccgccctcctcctcctcctcctccacctcctccacctcctccacttcctcctcctccacgacggcccccctcctccccaaggctgcgcgcgagaagccggaggcgccggccgagcctccaggccccgggcccgggtcaggcgcgcacccgggcggcagcgcccggccggacgccaaggaggagcagcagcagcagctgcggcgcaagatcaacagccgcgagcggaagcgcatgcaggacctgaacctggccatggacgccctgcgcgaggtcatcctgccctactcagcggcgcactgccagggcgcgcccggccgcaagctctccaagatagccacgctgctgctcgcccgcaactacatcctactgctgggcagctcgctgcaggagctgcgccgcgcgctgggcgagggcgccgggcccgccgcgccgcgcctgctgctggccgggctgcccctgctcgccgccgcgcccggctccgtgttgctggcgcccggcgccgtaggaccccccgacgcgctgcgccccgccaagtacctgtcgctggcgctggacgagccgccgtgcggccagttcgctctccccggcggcggcgcaggcggccccggcctctgcacctgcgccgtgtgcaagttcccgcacctggtcccggccagcctgggcctggccgccgtgcaggcgcaattctccaagtgagggcgggcctgggcctggggcgcgacctcggcccggcctcccttcgctcagcttctccgcgcccctgctccctgcgtctgggagagcgaggccgagcaaggaaagcatttcgaaccttccagtccagaggaagggactgtcgggcacccccttccccgcccccacccctgggacgttaaagtgaccagagcggatgttcgatggcgcctcggggcagtttggggttctgggtcggttccagcggctttaggcagaaagtgctcgctctcacccagcacatctctctccttgtccctggagttgcgcgcttcgcggggccgatgtagaacttagggcgccttgccgtggttggcgcgccccgggtgcagcgagaggccatccccgagcgctatctccccggagcggagcacgccggctcccagtactaggggctgcgctcgagcagtggcgggggcggaggggtggttcttttccttctcctccgccagaggccacgggcgcccttgttcccgccggccaggtcctatcaaaggaggctgccggaactcaagaggcagaaaaagaccagttaggcggtgcagacggtctgggacgtggcagacggacggaccctcggcggacaggtggtcggcgtcggggtgcggtgggtaggggcgaggacaacgcagggtgcgctgggttgggacgtgggtccacttttgtagaccagctgtttggagagctgtatttaagactcgcgtatccagtgttttgtcgcagagagttttcgctcttaaatcctgggggtttcttagaaagcaacttagaactcgagattcacctttcgtttccctttccccaaaagtagcgtaaccaacatttaagcttgcttaaaaacgaaaaccaaccgccttgcatccagtgttcccgatttactaaaataggtaaccaggcgtctcacagtcgccgtcctgtcaagagcgctaatgaacgttctcattaacacgcaggagtaccgggagccctgaaccgcccgctgctcggcggatcccagctgcggtggcgacggcgggaaggcgctttccgctgttcctcagcgggccgggcccttgaccagcgcggcccgcaggtcttccttctcgccgtcttgcagttgaagagctacatacgtagtcagtttcgatttgttacagacgttaacaaattcctttacccaaggttatgctatgacctttccgcagtttactttgattttctatgtttaaggttttggttgttggtagtagccgaatttaactggcactttattttacttctaaccttgtttcctgacggtgtacagaatcaacaaaataaaacatttaaagtctgattttttaaaaaaaaaaaaaaa

SEQ ID NO: 47 - DNA, 1936 bp, Homo sapiens
gcagaggcgcaggtagatggagttggggagttgcctggagggcgggagggaggcggcggaggaagagggcgagcctgaggtgaaaaagcggcgacttctgtgtgtgaggtttgcctcggtcgcaagctgcgatgccgcagtggctcagtgcttcctggccgagaacgactgggagatggaaagggctctgaactcctacttcgagcctccggtggaggagagcgccttggaacgccgacctgaaaccatctctgagcccaagacctatgttgacctaaccaatgaagaaacaactgattccaccacttctaaaatcagcccatctgaagatactcagcaagaaaatggcagcatgttctctctcattacctggaatattgatggattagatctaaacaatctgtcagagagggctcgaggggtgtgttcctacttagctttgtacagcccagatgtgatatttctacaggaagttattcccccatattatagctacctaaagaagagatcaagtaattatgagattattacaggtcatgaagaaggatatttcacagctataatgttgaagaaatcaagagtgaaattaaaaagccaagagattattccttttccaagtaccaaaatgatgagaaaccttttatgtgtgcatgtgaacgtgtcaggaaatgagctttgccttatgacatcccatttggagagcaccagagggcatgctgcggaacgaatgaatcagttaaaaatggttttaaagaaaatgcaagaggctccagagtcagctacagttatatttgcaggagatacaaatctaagggatcgagaggttaccagatgtggtggtttacccaacaacattgtggatgtctgggagtttttgggcaaacctaaacattgccagtatacatgggatacacaaatgaactctaatcttggaataactgctgcttgtaaacttcgttttgatcgaatatttttcagagcagcagcagaagagggacacattattccccgaagtttggaccttcttggattagaaaaactggactgtggtagatttcctagtgatcactggggtcttctgtgcaacttagatataatattgtaaaatgcttttcaagtgtgggttttgccctgattgttgcaaatacaatttccaccttctggaaaggtaggtttgctgtggaggaaataatgtactagatcattgtcacagaaaaaccaactatgatttatggttgtgttttcagaattcaacattaaagattaatgtttatttaaacgaacacattcctgcattcaggatgtgaggccatttaataaaaagggcacaaagcctgtcagagttttcaacggtgcttatagctgccagctggattccaaacaggtaccccattgtctctgagctaatgtttatatttttccattcaggcaccgaaatagttaatatttaaaataagtcttcaaaagaaaacataagagattattgagttcttgggactggatcctttatttcataagttcagatcatcttaaatgaaaatgccatgattatctgcagttaagtagatgacagctattctacatcagacttgatttttgtcagctaattacataattggtaagctataattgaaaccttatggcttaaaattccttaactcctttttgattcatgtttgtagtcatgttgtcaacagaggcaaagttaagcttgatgatggttaaaatcggtttgatagcaccatgggacatttttctaacaaaaataaatgcatgaagagacatagccttttagttttgctaattgtgaaatggaaatgctttacaggaagtaaatgcaaattacttttaagtgtgctttaaagaaaaatattttccccacaagagaaatttaaataaagaattttatttgtttaaaaaaaaaaaaaaaaaaaaa

SEQ ID NO: 48 - DNA, 494 bp, Homo sapiens
tgtggttgctcgtagtgagttgcgctcgctatgtctggacgtggcaagcagggaggcaaagcccgcgctaaggccaagactcgctcttctagggccggtctccagttccccgtgggccgagtgcaccgcctgctccgcaaaggcaactatgccgagcgggtcggggccggcgcgccggtgtatctggcagcggtgctggagtacctgaccgccgagatcctggaactggcgggcaacgcggcccgcgacaacaagaagacccgcatcatcccgcgtcatctccaactggccatccgcaacgacgaggagctcaacaagctgctgggcaaagtcaccatcgcacagggcggtgtcctgcccaacattcaggccgtgctactgcccaaaaagactgagagccaccacaaggcgaagggcaagtaactatctgtactagtttgtggcagctcaagtaaaatcgagtccaaaccaacggctcttttcagggccaccca

SEQ ID NO: 49 - DNA, 1152 bp, Homo sapiens
tcagagttcacgaggcagccgaggaagaggaggcttgaggcccagggtgggcaccagccagccatggccacagccgagaccgccttgccctccatcagcacactgaccgccctgggccccttcccggacacacaggatgacttcctcaagtggtggcgctccgaagaggcgcaggacatgggcccgggtcctcctgaccccacggagccgcccctccacgtgaagtctgaggaccagcccggggaggaagaggacgatgagaggggcgcggacgccacctgggacctggatctcctcctcaccaacttctcgggcccggagcccggtggcgcgccccagacctgcgctctggcgcccagcgaggcctccggggcgcaatatccgccgccgcccgagactctgggcgcatatgctggcggcccggggctggtggctgggcttttgggttcggaggatcactcgggttgggtgcgccctgccctgcgagcccgggctcccgacgccttcgtgggcccagccctggctccagccccggcccccgagcccaaggcgctggcgctgcaaccggtgtacccggggcccggcgccggctcctcgggtggctacttcccgcggaccgggctttcagtgcctgcggcgtcgggcgccccctacgggctactgtccgggtaccccgcgatgtacccggcgcctcagtaccaagggcacttccagctcttccgcgggctccagggacccgcgcccggtcccgccacgtccccctccttcctgagttgtttgggacccgggacggtgggcactggactcggggggactgcagaggatccaggtgtgatagccgagaccgcgccatccaagcgaggccgacgttcgtgggcgcgcaagaggcaggcagcgcacacgtgcgcgcacccgggttgcggcaagagctacaccaagagctcccacctgaaggcgcatctgcgcacgcacacaggggagaagccatacgcctgcacgtgggaaggctgcggctggagattcgcgcgctcggacgagctgacccgccactaccggaaacacacggggcagcgccccttccgctgccagctctgcccacgtgctttttcgcgctctgaccacctggccttgcacatgaagcgccacctttga

SEQ ID NO: 50 - DNA, 1362 bp, Homo sapiens
agcaactccaaggacacagttcacagaaatttggttctcagccccaaaatactgattgaattggagacaattacaaggactctctggccaaaaacccttgaagaggccccgtgaaggaggcagtgaggagcttttgattgctgacctgtgtcgtaccaccccagaatgtgcactgggggctgtgccagatgcctgggggggaccctcattccccttgctttttttggcttcctggctaacatcctgttattttttcctggaggaaaagtgatagatgacaacgaccacctttcccaagagatctggtttttcggaggaatattaggaagcggtgtcttgatgatcttccctgcgctggtgttcttgggcctgaagaacaatgactgctgtgggtgctgcggcaacgagggctgtgggaagcgatttgcgatgttcacctccacgatatttgctgtggttggattcttgggagctggatactcgtttatcatctcagccatttcaatcaacaagggtcctaaatgcctcatggccaatagtacatggggctaccccttccacgacggggattatctcaatgatgaggccttatggaacaagtgccgagagcctctcaatgtggttccctggaatctgaccctcttctccatcctgctggtcgtaggaggaatccagatggttctctgcgccatccaggtggtcaatggcctcctggggaccctctgtggggactgccagtgttgtggctgctgtgggggagatggacccgtttaaacctccgagatgagctgctcagactctacagcatgacgactacaatttcttttcataaaacttcttctcttcttggaattattaattcctatctgcttcctagctgataaagcttagaaaaggcagttattccttctttccaaccagctttgctcgagttagaattttgttattttcaaataaaaaatagtttggccacttaacaaatttgatttataaatctttcaaattagttcctttttagaatttaccaacaggttcaaagcatacttttcatgatttttttattacaaatgtaaaatgtataaagtcacatgtactgccatactacttctttgtatataaagatgtttatatctttggaagttttacataaatcaaaggaagaaagcacatttaaaatgagaaactaagaccaatttctgtttttaagaggaaaaagaatgattgatgtatcctaagtattgttatttgttgtctttttttgctgccttgcttgagttgcttgtgactgatcttttgaggctgtcatcatggctagggttcttttatgtatgttaaattaaaacctgaattcagaggtaacgt

SEQ ID NO: 51 - DNA, 2088 bp, Homo sapiens
gaattcggcacgagcgcgcggcgaatctcaacgctgcgccgtctgcgggcgcttccgggccaccagtttctctgctttccaccctggcgccccccagccctggctccccagctgcgctgccccgggcgtccacgccctgcgggcttagcgggttcagtgggctcaatctgcgcagcgccacctccatgttgaccaagcctctacaggggcctcccgcgccccccgggacccccacgccgccgccaggaggcaaggatcgggaagcgttcgaggccgagtatcgactcggccccctcctgggtaaggggggctttggcaccgtcttcgcaggacaccgcctcacagatcgactccaggtggccatcaaagtgattccccggaatcgtgtgctgggctggtcccccttgtcagactcagtcacatgcccactcgaagtcgcactgctatggaaagtgggtgcaggtggtgggcaccctggcgtgatccgcctgcttgactggtttgagacacaggaaggcttcatgctggtcctcgagcggcctttgcccgcccaggatctctttgactatatcacagagaagggcccactgggtgaaggcccaagccgctgcttctttggccaagtagtggcagccatccagcactgccattcccgtggagttgtccatcgtgacatcaaggatgagaacatcctgatagacctacgccgtggctgtgccaaactcattgattttggttctggtgccctgcttcatgatgaaccctacactgactttgatgggacaagggtgtacagccccccagagtggatctctcgacaccagtaccatgcactcccggccactgtctggtcactgggcatcctcctctatgacatggtgtgtggggacattccctttgagagggaccaggagattctggaagctgagctccacttcccagcccatgtctccccagactgctgtgccctaatccgccggtgcctggcccccaaaccttcttcccgaccctcactggaagagatcctgctggacccctggatgcaaacaccagccgaggatgttacccctcaacccctccaaaggaggccctgcccctttggcctggtccttgctaccctaagcctggcctggcctggcctggcccccaatggtcagaagagccatcccatggccatgtcacagggatagatggacatttgttgacttggttttacaggtcattaccagtcattaaagtccagtattactaaggtaagggattgaggatcaggggttagaagacataaaccaagtttgcccagttcccttcccaatcctacaaaggagccttcctcccagaacctgtggtccctgattttggagggggaacttcttgcttctcattttgctaaggaagtttattttggtgaagttgttcccattttgagccccgggactcttattttgatgatgtgtcaccccacattggcacctcctactaccaccacacaaacttagttcatatgcttttacttgggcaagggtgctttccttccaataccccagtagcttttattttagtaaagggaccctttcccctagcctagggtcccatattgggtcaagctgcttacctgcctcagcccaggattttttattttgggggaggtaatgccctgttgttaccccaaggcttctttttttttttttttttttttgggtgaggggaccctactttgttatcccaagtgctcttattctggtgagaagaaccttaattccataatttgggaaggaatggaagatggacaccaccggacaccaccagacaataggatgggatggatggttttttgggggatgggctaggggaaataaggcttgctgtttgttttcctggggcgctccctccaattttgcagatttttgcaacctcctcctgagccgggattgtccaattactaaaatgtaaataatcacgtattgtggggaggggagttccaagtgtgccctccttttttttcctgcctggattatttaaaaagccatgtgtggaaacccactatttaataaaagtaatagaatcagaaaaaaaaaaaaaaaaaaa

SEQ ID NO: 52 - DNA, 735 bp, Homo sapiens
agtggttctccgcccctgccactgggccatggagactgtggcacagtagactgtagtgtgaggctcgcgggggcagtggccatggaggccgtgctgaacgagctggtgtctgtggaggacctgctgaagtttgaaaagaaatttcagtctgagaaggcagcaggctcggtgtccaagagcacgcagtttgagtacgcctggtgcctggtgcggacaaggtacaatgatgacatccgtaaaggcatcgtgctgctcgaggagctgctgcccaaagggagcaaggaggaacagcgggattacgtcttctacctggccgtggggaactaccggctcaaggaatacgagaaggccttaaagtacgtccgcgggttgctgcagacagagccccagaacaaccaggccaaggaactggagcggctcattgacaaggccatgaagaaagatggactcgtgggcatggccatcgtgggaggcatggccctgggtgtggcgggactggccggactcatcggacttgctgtgtccaagtccaaatcctgaaggagacgcgggagcccacggagaacgctccaggagggcctgtccatcctcgctgtcctttccctgttctccccctgccccccgtctctatcctctgtggccttcagctaatttctgctcccctgagattcgtccttcagccccatcatgtgctttgggatgagtgtaaataaaacggggctgtggcttgggaaa

SEQ ID NO: 53 - DNA, 2627 bp, Homo sapiens
gctgacgccttcgagcgcggcccggggcccggagcggccggagcagcccgggtcctgaccccggcccggctcccgctccgggctctgccggcgggcgggcgagcgcggcgcggtccgggccggggggatgtctcggcggacgcgctgcgaggatctggatgagctgcactaccaggacacagattcagatgtgccggagcagagggatagcaagtgcaaggtcaaatggacccatgaggaggacgagcagctgagggccctggtgaggcagtttggacagcaggactggaagttcctggccagccacttccctaaccgcactgaccagcaatgccagtacaggtggctgagagttttgaatccagaccttgtcaaggggccatggaccaaagaggaagaccaaaaagtcatcgagctggttaagaagtatggcacaaagcagtggacactgattgccaagcacctgaagggccggctggggaagcagtgccgtgaacgctggcacaaccacctcaaccctgaggtgaagaagtcttgctggaccgaggaggaggaccgcatcatctgcgaggcccacaaggtgctgggcaaccgctgggccgagatcgccaagatgttgccagggaggacagacaatgctgtgaagaatcactggaactctaccatcaaaaggaaggtggacacaggaggcttcttgagcgagtccaaagactgcaagcccccagtgtacttgctgctggagctcgaggacaaggacggcctccagagtgcccagcccacggaaggccagggaagtcttctgaccaactggccctccgtccctcctaccataaaggaggaggaaaacagtgaggaggaacttgcagcagccaccacatcgaaggaacaggagcccatcggtacagatctggacgcagtgcgaacaccagagcccttggaggaattcccgaagcgtgaggaccaggaaggctccccaccagaaacgagcctgccttacaagtgggtggtggaggcagctaacctcctcatccccgctgtgggttctagcctctctgaagccctggacttgatcgagtcggaccctgatgcttggtgtgacctgagtaaatttgacctccctgaggaaccatctgcagaggacagtatcaacaacagcctagtgcagctgcaagcgtcacatcagcagcaagtcctgccaccccgccagccttccgccctggtgcccagtgtgaccgagtaccgcctggatggccacaccatctcagacctgagccggagcagccggggcgagctgatccccatctcccccagcactgaagtcgggggctctggcattggcacaccgccctctgtgctcaagcggcagaggaagaggcgtgtggctctgtcccctgtcactgagaatagcaccagtctgtccttcctggattcctgtaacagcctcacgcccaagagcacacctgttaagaccctgcccttctcgccctcccagtttctgaacttctggaacaaacaggacacattggagctggagagcccctcgctgacatccaccccagtgtgcagccagaaggtggtggtcaccacaccactgcaccgggacaagacacccctgcaccagaaacatgctgcgtttgtaaccccagatcagaagtactccatggacaacactccccacacgccaaccccgttcaagaacgccctggagaagtacggacccctgaagcccctgccacagaccccgcacctggaggaggacttgaaggaggtgctgcgttctgaggctggcatcgaactcatcatcgaggacgacatcaggcccgagaagcagaagaggaagcctgggctgcggcggagccccatcaagaaagtccggaagtctctggctcttgacattgtggatgaggatgtgaagctgatgatgtccacactgcccaagtctctatccttgccgacaactgccccttcaaactcttccagcctcaccctgtcaggtatcaaagaagacaacagcttgctcaaccagggcttcttgcaggccaagcccgagaaggcagcagtggcccagaagccccgaagccacttcacgacacctgcccctatgtccagtgcctggaagacggtggcctgcggggggaccagggaccagcttttcatgcaggagaaagcccggcagctcctgggccgcctgaagcccagccacacatctcggaccctcatcttgtcctgaggtgttgagggtgtcacgagcccattctcatgtttacaggggttgtgggggcagagggggtctgtgaatctgagagtcattcaggtgacctcctgcagggagccttctgccaccagcccctccccagactctcaggtggaggcaacagggccatgtgctgccctgttgccgagcccagctgtgggcggctcctggtgctaacaacaaagttccacttccaggtctgcctggttccctccccaaggccacagggagctccgtcagcttctcccaagcccacgtcaggcctggcctcatctcagaccctgcttaggatgggggatgtggccaggggtgctcctgtgctcaccctctcttggtgcatttttttggaagaataaaattgcctctctctttg

SEQ ID NO: 54 - DNA, 1249 bp, Homo sapiens
ctgattttctctttggattcttccaaaatcagagtcagactactccctgtgccatgaacggagatgacacctttgcaaggagacccacggttggtgctcaaataccagagaagatacaaaaggccttcgatgatattgccaaatacttctctaaggaagagtgggaaaagatgaaagtctcggagaaaatcgtctatgtgtatatgaagagaaagtatgaggccatgactaaactaggtttcaaggccatcctcccatctttcatgcgtaataaacgggtcacagacttccaggggaatgattttgataatgaccctaaccgtgggaatcaggttcaacgtcctcagatgactttcggcaggctccagggaatcttcccgaagatcatgcccaagaagccagcagaggaaggaaatgtttcgaaggaagtgccagaagcatctggcccacaaaacgatgggaaacagctgtgccccccgggaaaaccaactacctctgagaagattaacatgatatctggacccaaaaggggggaacatgcctggacccacagactgcgtgagagaaagcagctggtgatttatgaagagatcagcgatcctgaggaagatgatgagtaactccccttggggatatgacacatgcccatgatgagaagcagaacgtggtgacctttcacgaacatgggcatggctgtggacccctcgtcatcaggtgcatagcaagtgaaagcaagtgttcacaacagtgaaaagttgagcgtcatttttcttagtgtgccaagagtacgatattagcgtttccattgtattttcttgaagtgtgtcattctgttagatatgaacattttcactgatgagcaagacatacttaatgcatattttggtttgtgtatccatgcacctaccttagaaaacaagtattgtcagttacctctgcatggaacagcattaccctcctctctccctagatgtgactactgagggcagttctgagtgtttaatttcagattttttcctctgcatttacacacacacacaaaccacaccacacacacacacacacacacacacacacacaccaagtaccagtataagcatctcccatctgcttttcccattgccatgcgtcctggtcaggcttccctcactctgtttcctggtcagcatgtactcccctcatccgattcccctgtagcagtcactgacagtaaataaacctttgcaaacgttaaaaaaaaaaaaaaaaa

SEQ ID NO: 55 - DNA, 1949 bp, Homo sapiens
atgacgcgagaccccgcccccgcagcgcccgcttccaagatggcggcagcgatgcctgcccggctgttggggtggcggtgacgacaggcagcaaaagaccagctggtcccagattcgctgctggagtgctggatggagcctttctctgccctctgtgacatttccaattttagataatgcctcacatctctgtccccccgggaccccctggagcccccatgatccctaagaagacagcttgaacctagatctcacccccaggatgttgcggaggctgctggagcggccttgcacgctggccctgcttgtgggctcccagctggctgtcatgatgtacctgtcactggggggcttccgaagtctcagtgccctatttggccgagatcagggaccgacatttgactattctcaccctcgtgatgtctacagtaacctcagtcacctgcctggggccccagggggtcctccagctcctcaaggtctgccctactgtccagaacgatctcctctcttagtgggtcctgtgtcggtgtcctttagcccagtgccatcactggcagagattgtggagcggaatccccgggtagaaccagggggccggtaccgccctgcaggttgtgagccccgctcccgaacagccatcattgtgcctcatcgtgcccgggagcaccacctgcgcctgctgctctaccacctgcaccccttcttgcagcgccagcagcttgcttatggcatctatgtcatccaccaggctggaaatggaacatttaacagggcaaaactgttgaacgttggggtgcgagaggccctgcgtgatgaagagtgggactgcctgttcttgcacgatgtggacctcttgccagaaaatgaccacaatctgtatgtgtgtgacccccggggaccccgccatgttgccgttgctatgaacaagtttggatacagcctcccgtacccccagtacttcggaggagtctcagcacttactcctgaccagtacctgaagatgaatggcttccccaatgaatactggggctggggtggtgaggatgacgacattgctaccagggtgcgcctggctgggatgaagatctctcggccccccacatctgtaggacactataagatggtgaagcaccgaggagataagggcaatgaggaaaatccccacagatttgacctcctggtccgtacccagaattcctggacgcaagatgggatgaactcactgacataccagttgctggctcgagagctggggcctctttataccaacatcacagcagacattgggactgaccctcggggtcctcgggctccttctgggccacgttacccacctggttcctcccaagccttccgtcaagagatgctgcaacgccggcccccagccaggcctgggcctctatctactgccaaccacacagccctccgaggttcacactgactcctccttcctgtctaccttaatcatgaaaccgaattcatggggttgtattctccccaccctcagctcctcactgttctcagagggatgtgagggaactgaactctggtgccgtgctagggggtaggggcctctccctcactgctggactggagctgggctcctgtagacctgaggggtccctctctctagggtctcctgtagggcttatgactgtgaatccttgatgtcatgattttatgtgacgattcctaggagtccctgcccctagagtaggagcagggctggaccccaagcccctccctcttccatggagagaagagtgatctggcttctcctcggacctctgtgaatatttattctatttatggttcccgggaagttgtttggtgaaggaagcccctccctgggcattttctgcctatgctggaatagctccctcttctggtcctggctcagggggctgggattttgatatattttctaataaaggactttgtctcgc

SEQ ID NO: 56 - DNA, 470 bp, Homo sapiens
gttcctccatttatcgtttcttcgtcatgtcgggacgcggcaagcagggaggcaaagctcgcgccaaagccaagacccgctcttctcgtgccggtctccagttccccgtgggccgagtgcaccgactgctccgcaagggcaactatgctgagcgggtcggggccggcgcgccggtgtacctggcggcggtgctggagtacctgactgccgagatcctggagctggcgggcaacgccgcccgcgacaacaagaagacccgcattatcccgcgccacttgcagctggccatccgcaacgacgaggagctcaacaagctgctgggcaaagtaaccatcgctcagggtggtgtcctgcccaacatccaggctgtgctactgcccaagaagaccgagagtcaccacaaggccaaaggcaaataatgtctccatagaatcactttccaatacaacggctcttttcagagccaccta

SEQ ID NO: 57 - DNA, 1120 bp, Homo sapiens
acttcttcgcaccagggaagccccacccaccagaacgccaagatgtccagcaagcgggccaaagccaaggccaccaagaagcggccacagcgggccacatccaatgtcttcgcaatgtttgaccagtcccagatccaggagtttaaggaggctttcaacatgattgaccagaaccgtgatggcttcattgacaaggaggacctgcacgacatgctggcctcgctggggaagaaccccacagacgaatacctggagggcatgatgagcgaggccccggggccatacaacttcaccatgttcctcaccatgtttggggagaagctgaacggcacggaccccgaggatgtgattcgcaacgcctttgcctgcttcgacgaggaatcctcaggtttcatccatgaggaccacctccggaagctgctcaccaccatgggtgaccgcttcacagatgaggaagtggacgagatgtaccgggaggcacccgttgataagaaaggcaacttcaactacgtggagttcacccgcatcctcaaacatggcgccaaggataaacacgactaggccatccccagccccctgacacccagcccccgccagtcacccctccccgcacacacccgtccataccagctccctgcccatgaccctcgctcagggatccccctttgagggttagggtcccagttcccagtggaagaaacaggccaggagagtgcgtgccgagctgaggcagatgttcccacagtgaccccagagccctgggctatagtctctgacccctccaaggaaagaccaccttctggggacatgggctggagggcaggacctagaggcaccaagggaaccgcattccggggctgttccccgaggaggaagggaagcctctgtgtgccccccaggaggaagaggccctgagtcctgggatcagacaccccttcacgtgtatcccacacaaatgcaagctcaccaaggtcccctctcagtccccttccctacaccctgacgccagatgccgcacacccaacgccaccagccatgggagtgtgctcaggagtcgcggggcagacgtgacatctgtccagagggggcagaatctccaatagaggactgagacaacatg

SEQ ID NO: 58 - DNA, 1497 bp, Homo sapiens
accaacctcttcgaggcacaaggcacaacaggctgctctgggattctcttcagccaatcttcattgctcaagtgtctgaagcagccatggcagaagtacctgagctcgccagtgaaatgatggcttattacagtggcaatgaggatgacttgttctttgaagctgatggccctaaacagatgaagtgctccttccaggacctggacctctgccctctggatggcggcatccagctacgaatctccgaccaccactacagcaagggcttcaggcaggccgcgtcagttgttgtggccatggacaagctgaggaagatgctggttccctgcccacagaccttccaggagaatgacctgagcaccttctttcccttcatctttgaagaagaacctatcttcttcgacacatgggataacgaggcttatgtgcacgatgcacctgtacgatcactgaactgcacgctccgggactcacagcaaaaaagcttggtgatgtctggtccatatgaactgaaagctctccacctccagggacaggatatggagcaacaagtggtgttctccatgtcctttgtacaaggagaagaaagtaatgacaaaatacctgtggccttgggcctcaaggaaaagaatctgtacctgtcctgcgtgttgaaagatgataagcccactctacagctggagagtgtagatcccaaaaattacccaaagaagaagatggaaaagcgatttgtcttcaacaagatagaaatcaataacaagctggaatttgagtctgcccagttccccaactggtacatcagcacctctcaagcagaaaacatgcccgtcttcctgggagggaccaaaggcggccaggatataactgacttcaccatgcaatttgtgtcttcctaaagagagctgtacccagagagtcctgtgctgaatgtggactcaatccctagggctggcagaaagggaacagaaaggtttttgagtacggctatagcctggactttcctgttgtctacaccaatgcccaactgcctgccttagggtagtgctaagaggatctcctgtccatcagccaggacagtcagctctctcctttcagggccaatccccagcccttttgttgagccaggcctctctcacctctcctactcacttaaagcccgcctgacagaaaccacggccacatttggttctaagaaaccctctgtcattcgctcccacattctgatgagcaaccgcttccctatttatttatttatttgtttgtttgttttattcattggtctaatttattcaaagggggcaagaagtagcagtgtctgtaaaagagcctagtttttaatagctatggaatcaattcaatttggactggtgtgctctctttaaatcaagtcctttaattaagactgaaaatatataagctcagattatttaaatgggaatatttataaatgagcaaatatcatactgttcaatggttctgaaataaacttctctgaag

SEQ ID NO: 59 - DNA, 1237 bp, Homo sapiens
agcgtgggtaaaagcaaaagcaacagctcaagcagcctccttggagaaaacctgaaaattcaacttgttcaagagaaggtcttgtacgtgcctaagttctagagcctcctgacgtgagcatggctgagagtgaggaccgctccctgaggatcgttctggtagggaaaactggaagtgggaaaagtgcaacagcgaacaccatccttggagaggaaatctttgattctagaattgctgcccaagctgttaccaagaactgtcaaaaagcatcccgggaatggcaggggagagaccttcttgttgtagacactccagggctctttgacaccaaggagagcctggacaccacctgcaaggaaatcagccgctgcatcatctcctcctgcccagggccccatgctattgtcctagttctgctgctgggccgctacacagaggaggagcagaaaaccgttgcattgatcaaggctgtctttgggaagtcagccatgaagcacatggtcatcttgttcactcgcaaagaagagttggagggccagagcttccatgacttcatagcagatgcggatgtgggcctaaaaagcatcgtcaaggagtgcgggaaccgctgctgtgcctttagcaacagcaagaaaaccagtaaggcagagaaggaaagtcaagtgcaggagttggtggagctgatagagaaaatggtgcagtgcaacgaaggggcttacttttctgatgacatatacaaggacacagaggaaaggctgaaacaacgggaagaggttttgaggaaaatctacactgaccaattaaatgaagaaattaaactagtagaagaggataagcataaatcagaggaagaaaaggagaaagaaattaaattactaaaattaaaatatgatgaaaaaataaaaaatataagggaagaagctgagagaaatatatttaaagatgtttttaataggatttggaagatgctttcagaaatatggcataggtttttgtcgaaatgtaagttttattcttcctaatttactgtgatttgttaatggatgaattgtattttgcaaagatagttagagaaatacctccttccccttagctttattaaggtatcattgataaataaaaataaaatatgtttaatgtatataatgtgatttttaaatatatatatatatatacacacattgtgaaataatgaaataaaggtaattaacacatctaaaacaaaaaaaaaaaaaaa

SEQ ID NO: 60 - DNA, 2397 bp, Homo sapiens
tttttagttctgacttaggccaaaatagaaaaaaagaaagtatgttcagaaggcaaatggtcatgagatcaaaggccaagggaccccgacagggcaggcgcagagctcctgcttggggcttgggtggggtgtttgtgggggttattctgctccgccccccggaaaggccaggagcccttcggattggcgtcttgctgagctcctgctgccccctgctggtttcgcggcactccctggtcctcagaaatgtagacaggatggtcaaatggaatcccatctcccctctctctcttcattcacttaaaattacctctcccatacggactgaaagtggcttgagtgataatagagaagttgaagctgcttttcagcctaaattatctccagaacggcttcttgttcttcattagaagagatgcgcttctcaggtttccaggtgagccggatagccctggctgtaggagtccagagagaatagttccttctctggtgtctctctcttcacgaagccaagaggggatctcatgtagggacccttgaataaaccatgcccgctggttaattccacatgcttttcatgtcttgcagttcagtgaattctacagtcttggtgaagaacacgaagaagactaatccagagataaaagaaaaaccctgccattttgaaagatgtgaaggggaggtgaacacacgcttcagcctaaaacactaagtagatgcaggcctgggccgttctcatacccccgggaaccatatcttacccattgtatgtcgcagcttgcaggccagtgcttggcacagagcagggactcaggaagcctttgtcactaaagtaagagcctctgcggagtacagtgcatggggtcggctgggccagccccaggcagcagatcctggtattgggctgaggaaagagcactgcgcttggagtcagtaagatctgccacctccctgagtctcatcagcaaaatgaggataaagataaagatactatagttgcccagcctgcttgacagggttgttgtaaggttcacataagatgatgatatgcaaatgctttgtaatctaggaggtgctatttgtctaaagtctaatggagaattataatacatccaggagttaaggagttctaatgcttaaaatgaaatagtctaagatcttagcaagaaaggattaagaaggacttttctctccatattgattttgtaatggagttataaataattgcttctagagactgagaaattgattggttttctttaactcctattctttcttttctttctttaatttttaaaaaactctttgaatagttacctttctctattttgggctgtttttgtcccaagagtaggattttttcccagtagagtgcagtggtccaagaatgggccactggatgatactgctttaccaacgagtgacaggaccatgaacctcacagttgtgaggttcaatgagggctggccctgccacataaatcctctgagggagatgatgacaattcactgctgattaatgccattctgcctttactgtaattagaaggaaataaccccagaatacaaggaatttagcaagataaggaacccctgctgctacctaaacatccatctaaacaaagatgtttggcttttgaagcaaagagtttggttctcaagactgtgttctttgacagttaattttcaagaagactgaagactgaattatcattgttgagaattctctaggtctcagtaaccctctgaaccagcagtttgggtggtcgatgcccagcaaataggagtgggtggccttttctctggtgtataagattcatctaatttttaggaatttttgtaccattttccccctctagaaacacatttactccccaataattgtacgggaggtgatcgaggaagaagaaccaagtgaaaaatcagaggccacctacatgaccatgcacccagtttggccttctctgaggtcagatcggaacaactcacttgaaaaaaagtcaggtgggggaatgccaaaaacacagcaagccttttgagaagaatggagagtcccttcatctcagcagcggtggagactctctcctgtgtgtgtcctgggccactctaccagtgatttcagactcccgctctcccagctgtcctcctgtctcattgtttggtcaatacactgaagatggagaatttggagcctggcagagagactggacagctctggaggaacgggcctgctgaggggaggggagcatggacttggcctctggagtgggacactggccctgggaaccaggctgagctgagtggcctcaaaccccccgttggatcagaccctcctgtgggcagggttcttagtggatgagttactgggaagaatcagagataaaaaccaacccaaatc

SEQ ID NO: 61 - DNA, 1763 bp, Homo sapiens
tagctggattccagccattgctgcagctgctccacagcccttttcaggacccaaacaaccgcagccgctgttcccaggatggtgatccgtgtatatattgcatcttcctctggctctacagcgattaagaagaaacaacaagatgtgcttggtttcctagaagccaacaaaataggatttgaagaaaaagatattgcagccaatgaagagaatcggaagtggatgagagaaaatgtacctgaaaatagtcgaccagccacaggttaccccctgccacctcagattttcaatgaaagccagtatcgcggggactatgatgccttctttgaagccagagaaaataatgcagtgtatgccttcttaggcttgacagccccacctggttcaaaggaagcagaagtgcaagcaaagcagcaagcatgaaccttaagcactgtgctttaagcatcctgaaaaatgagtctccattgcttttataaaatagcagaattagctttgcttcaaaagaaataggcttaatgttgaaataatagattagttgggttttcacatgcaaacattcaaaatgaatacaaaattaaaatttgaacattatggtgattatggtgaggagaatgggatattaacataaaattatattaataagtagatatcgtagaaatagtgttgttacctgccaagccatcctgtatacaccaatgattttacaaagaaaacacccttccctccttctgccattactatggcaacttaagtgtatctgcagctctacattaaaaaggagaaagagaaataacctgtctctcattcctaagttgcctcattaattttcatgaacaagaatatgtacctttttgatgctatattactgcgattaaaaaagttcttgcaggtaatgtttatgtatagttaaacgttgtaatttcttatcgtaattataacattcccattctttgtagatgaaactctacatatgaaccacagattttctgagcttctaaatgtagcctttcattgcacatttcagtgatcagaatagatatccttttacacgcacaaaagcaatagattcattcagtggacaagttccttgtttaactacacagctatgatggaatcatatatccaagttccttgcctcagtgaaatatgcatatgtatatcatgaagtgggatgccaagtaagcttaaaatgcattctctagcaaagagattagacttttaaataactcttataaaacaggttggcgatcatttcccaagattggtttcccttgagtttttgttaaaacaaatcttagtagttttgcccgtttaaaacaactcacaatcgtaaatgctactattcctaagatatcttacctttttatttcagtttagccatgtattgtatgagtgtattagtctaagcagtgagaatcttttctatgcctctattccagcaaaaagtagaagtatcaaataaaaagggcaacttttaaaatattaagcctgaagacttctaaaaagacaagaaacatggcctaaataaccaacatagatttacatagtaagtttcacactaccttattaccaaaagcaaacacctcttactttaaactacattatcatgtatatctattgtatgctggtctttactttttgccaaaatcaacatataatgaagagatgcctttgtttgatgagattcaaacttgatgctatgctttaaaataaactcagtacttttagaaacataaaaaaaaaaaaaaaaaaaaaaaaaa

SEQ ID NO: 62 - DNA, 1134 bp, Homo sapiens
cgacccctcgaggggcccagccttggaagggtaactggaccgctgccgcctggttgcctgggccagaccagacatgcctgctgctccttccggcttaggaggagcacgcgtcccgctcgggcgcactctccagccttttcctggctgaggaggggccgagcctccggtagggcgggggccggatgaggcgggacctcaggcccggaaaactgcctgtgccacgtgacccgccgccggccagttaaaaggaggcgcctgctggcctccccttacagtgcttgttcggggcgctccgctggcttcttggacaattgcgccatgtgtgctgctcggctagcggcggcggcggcccagtcggtgtatgccttctcggcgcgcccgttggccggcggggagcctgtgagcctgggctccctgcggggcaaggtactacttatcgagaatgtggcgtccctctgaggcaccacggtccgggactacacccagatgaacgagctgcagcggcgcctcggaccccggggcctggtggtgctcggcttcccgtgcaaccagtttgggcatcaggagaacgccaagaacgaagagattctgaattccctcaagtacgtccggcctggtggtgggttcgagcccaacttcatgctcttcgagaagtgcgaggtgaacggtgcgggggcgcaccctctcttcgccttcctgcgggaggccctgccagctcccagcgacgacgccaccgcgcttatgaccgaccccaagctcatcacctggtctccggtgtgtcgcaacgatgttgcctggaactttgagaagttcctggtgggccctgacggtgtgcccctacgcaggtacagccgccgcttccagaccattgacatcgagcctgacatcgaagccctgctgtctcaagggcccagctgtgcctagggcgcccctcctaccccggctgcttggcagttgcagtgctgctgtctcgggggggttttcatctatgagggtgtttcctctaaacctacgagggaggaacaccttgatcttacagaaaataccacctcgagatgggtgctggtcctgttgatcccagtctctgccagaccaaggcgagtttccccactaataaagtgccgggtgtcagca

SEQ ID NO: 63 - DNA, 1233 bp, Homo sapiens
gaattccgccaagcggggacctcaggatggaaaccagcagcctgcaccgcccgagaaggtcggctgggtccggaaattctgcgggaaagggattttcagggagatttggaaaaaccgctatgtggtgctgaaaggggaccagctctacatctctgagaaggaggtaaaagatgagaaaaatattcaagaggtatttgacctgagtgactatgagaagtgtgaagagctccggaagtccaagagcaggagcaagaaaaatcatagcaagtttactcttgcccactccaaacagcccggtaacacggcacccaacctgatcttcctggcagtgagtccagaagagaaggaatcgtggatcaatgccctcaactctgccatcacccgagccaagaaccgtatcttggatgaggtcaccgttgaggaggacagctatcttgcccatcccactcgagacagggcaaaaatccagcactcccgccgccccccaacaaggggacacctaatggctgtggcttccacctctacctcggatgggatgctgaccttggacttgatccaagaggaagacccttcccctgaggaaccaacctcttgtgctgagagctttcgggttgacctggacaagtctgtggcccagctggcagggagccggcggagagcggactcagaccgcatccagccctccgcagaccgggcaagcagtctctcccgaccttgggaaaaaacagacaaaggggccacctacaccccccaggcacccaagaagttgacgcccacagagaaaggccgctgcgcctccctggaggagatcctatctcagcgggatgctgcctctgcccgcaccctccagctgcgggctgaggaacccccaacccctgccctccccaacccggggcagctgtcccggatccaggacctggtagcaaggaaactggaggagactcaggagcttctggcagaggttcagggactgggagatgggaagcgaaaggccaaggacccccctcggtctccgccggattctgagtcagagcagctgctgctggagacggaacggctgctgggagaggcatcatcgaattggagccaggcaaagagggtgctgcaggaggtcagggagctgagagacctgtacagacagatggacctgcagaccccggactcccacctcagacagaccaccccgcacagtcagtaccggaagagcctgatgtgagggcagggtggggtctg

SEQ ID NO: 64 - DNA, 2396 bp, Homo sapiens
ggcacgagggctgtgcgggtggcggccggcgcgcggtggggcatggcgggttcgcggggtgcggggcgcacggcggcgccgagcgtgcggccggagaagcggcggtctgagcccgaactggagcctgagcccgagccggagccccccctcctctgcacctctcctctcagccacagcaccggcagcgattctggcgtctccgacagcgaggagagtgtgttctcaggcctggaagattccggcagtgacagcagtgaggatgatgacgaaggcgacgaggagggagaggacggagcccttgatgacgagggccacagtgggattaaaaagaccactgaggagcaggtgcaggccagcactccttgcccgaggacagagatggcgagcgcccggattggggatgagtatgcggaggacagctctgatgaggaggacatccggaacacggtgggcaacgtgcccttggagtggtacgatgacttcccccacgtgggctacgacctggatggcaggcgcatctacaagcccctgcggacccgggatgagctggaccagttcctggacaagatggacgatcctgactactggcgcaccgtgcaggacccgatgacagggcgggacctgagactgacggatgagcaggtggccctggtgcggcggctgcagagtggccagtttggggatgtgggcttcaacccctatgagccggctgtcgacttcttcagcggggacgtcatgatccacccggtgaccaaccgcccggccgacaagcgcagcttcatcccctccctggtggagaaggagaaggtctctcgcatggtgcacgccatcaagatgggctggatccagcctcgccggccccgagaccccacccccagcttctatgacctgtgggcccaggaggaccccaacgccgtgctcgggcgccacaagatgcacgtacctgctcccaagctggccctgccaggccacgccgagtcgtacaacccaccccctgaatacctgctcagcgaggaggagcgcttggcgtgggaacagcaggagccaggcgagaggaagctgagctttttgccacgcaagttcccgagcctgcgggccgtgcctgcctacggacgcttcatccaggaacgcttcgagcgctgccttgacctgtacctgtgcccacggcagcgcaagatgagggtgaatgtagaccctgaggacctcatccccaagctgcctcggccgagggacctgcagcccttccccacgtgccaggccctggtctacaggggccacagtgaccttgtccggtgcctcagtgtctctcctgggggccagtggctggtttcaggctctgacgacggctccctgcggctctgggaggtggccactgcccgctgtgtgaggactgttcccgtggggggcgtggtgaagagtgtggcctggaaccccagccccgctgtctgcctggtggctgcagccgtggaggactcggtgctgctgctgaacccagctctgggggaccggctggtggcgggcagcacagatcagctgttgagcgccttcgtcccgcctgaggagccccccttgcagccggcccgctggctggaggcctcagaggaggagcgccaagtgggcctgcggctgcgcatctgccacgggaagccagtgacgcaggtgacctggcacgggcgtggggactacctggccgtggtgctggccacccaaggccacacccaggtgctgattcaccagctgagccgtcgccgcagccagagtccgttccgccgcagccacggacaggtgcagcgagtggccttccaccctgcccggcccttcctgttggtggcgtcccagcgcagcgtccgcctctaccacctgctgcgccaggagctcaccaagaagctgatgcccaactgcaagtgggtgtccagcctggcggtgcaccctgcaggtgacaacgtcatctgtgggagctacgatagcaagctggtgtggtttgacctggatctttccaccaagccatacaggatgctgagacaccacaagaaggctctgcgggctgtggccttccacccgcggtacccactctttgcgtcaggctcggacgacggcagtgtcatcgtctgccatggcatggtgtacaatgaccttctgcagaaccccttgctggtgcccgtcaaggtgctgaagggacacgtgctgacccgagatctgggagtgctggacgtcatcttccaccccacccagccgtgggtcttctcctcgggggcagacgggactgtccgcctcttcacctagctgttctgcctgcctggggctggggtggtcgtgctgaagtcaacagagcctttaccctgtgcaaaaaaaaaaaaaaaaaaaaaaaaaaaaaaaaaaaaaaaaaaaaaaaaaaa

SEQ ID NO: 65 - DNA, 1048 bp, Homo sapiens
aggagctggggccatcaaggcggaccatgtgtcaacttatgccgcgtttgtacagacgcatagaccaacaggggagtttatgtttgaatttgatgaagatgagatgttctatgtggatctggacaagaaggagaccgtctggcatctggaggagtttggccaagccttttcctttgaggctcagggcgggctggctaacattgctatattgaacaacaacttgaataccttgatccagcgttccaaccacactcaggccaccaacgatccccctgaggtgaccgtgtttcccaaggagcctgtggagctgggccagcccaacaccctcatctgccacattgacaagttcttcccaccagtgctcaacgtcacgtggctgtgcaacggggagctggtcactgagggtgtcgctgagagcctcttcctgcccagaacagattacagcttccacaagttccattacctgacctttgtgccctcagcagaggacttctatgactgcagggtggagcactggggcttggaccagccgctcctcaagcactgggaggcccaagagccaatccagatgcctgagacaacggagactgtgctctgtgccctgggcctggtgctgggcctagtcggcttcatcgtgggcaccgtcctcatcataaagtctctgcgttctggccatgacccccgggcccaggggaccctgtgaaatactgtaaaggtgacaaaatatctgaacagaagaggacttaggagagatctgaactccagctgccctacaaactccatctcagcttttcttctcacttcatgtgaaaactactccagtggctgactgaattgctgacccttcaagctctgtccttatccattacctcaaagcagtcattccttagtaaagtttccaacaaatagaaattaatgacactttggtagcactaatatggagattatcctttcattgagccttttatcctctgttctcctttgaagagcccctcactgtcaccttcccgagaataccctaagaccaataaatacttcagtatttcag

SEQ ID NO: 66 - DNA, 1285 bp, Homo sapiens
ggggcccagggccctcctatggaccctgcccgctcccctcccattgtccacggctgtccgcccacccccattctccaagcttcagccccctccttagttcggcatctgcacagcactgaagaacctgggaatcagaccctgagaccctgagcaatcccaggtccagcgccagccctatcatgaccaaggagtatcaagaccttcagcatctggacaatgaggagagtgaccaccatcagctcagaaaagggccacctcctccccagcccctcctgcagcgtctctgctccggacctcgcctcctcctgctctccctgggcctcagcctcctgctgcttgtggttgtctgtgtgatcggatcccaaaactcccagctgcaggaggagctgcggggcctgagagagacgttcagcaacttcacagcgagcacggaggcccaggtcaagggcttgagcacccagggaggcaatgtgggaagaaagatgaagtcgctagagtcccagctggagaaacagcagaaggacctgagtgaagatcactccagcctgctgctccacgtgaagcagttcgtgtctgacctgcggagcctgagctgtcagatggcggcgctccagggcaatggctcagaaaggacctgctgcccggtcaactgggtggagcacgagcgcagctgctactggttctctcgctccgggaaggcctgggctgacgccgacaactactgccggctggaggacgcgcacctggtggtggtcacgtcctgggaggagcagaaatttgtccagcaccacataggccctgtgaacacctggatgggcctccacgaccaaaacgggccctggaagtgggtggacgggacggactacgagacgggcttcaagaactggaggccggagcagccggacgactggtacggccacgggctcggaggaggcgaggactgtgcccacttcaccgacgacggccgctggaacgacgacgtctgccagaggccctaccgctgggtctgcgagacagagctggacaaggccagccaggagccacctctcctttaatttatttcttcaatgcctcgacctgccgcaggggtccgggattgggaatccgcccatctgggggcctcttctgctttctcgggaattttcatctaggattttaagggaaggggaaggatagggtgatgttccgaaggtgaggagcttgaaacccgtggcgctttctgcagtttgcaggttatcattgtgaactttttttttttttaagagtaaaaagaaatatacctaaa

SEQ ID NO: 67 - DNA, 1820 bp, Homo sapiens
ggggatgcaactaagttgctgagacaagggaagagagatgaggaaccagagcttgtagaaaccactttaatcatatccaggagtttgcaagaaacaggtgcttaacactaattcacctcctgaacaagaaaaatgggctgtgaccggaactgtgggctcatcgctggggctgtcattggtgctgtcctggctgtgtttggaggtattctaatgccagttggagacctgcttatccagaagacaattaaaaagcaagttgtcctcgaagaaggtacaattgcttttaaaaattgggttaaaacaggcacagaagtttacagacagttttggatctttgatgtgcaaaatccacaggaagtgatgatgaacagcagcaacattcaagttaagcaaagaggtccttatacgtacagagttcgttttctagccaaggaaaatgtaacccaggacgctgaggacaacacagtctctttcctgcagcccaatggtgccatcttcgaaccttcactatcagttggaacagaggctgacaacttcacagttctcaatctggctgtggcagctgcatcccatatctatcaaaatcaatttgttcaaatgatcctcaattcacttattaacaagtcaaaatcttctatgttccaagtcagaactttgagagaactgttatggggctatagggatccatttttgagtttggttccgtaccctgttactactacagttggtctgttttatccttacaacaatactgcagatggagtttataaagttttcaatggaaaagataacataagtaaagttgccataatcgacacatataaaggtaaaaggaatctgtcctattgggaaagtcactgcgacatgattaatggtacagatgcagcctcatttccaccttttgttgagaaaagccaggtattgcagttcttttcttctgatatttgcaggtcaatctatgctgtatttgaatccgacgttaatctgaaaggaatccctgtgtatagattcgttcttccatccaaggcctttgcctctccagttgaaaacccagacaactattgtttctgcacagaaaaaattatctcaaaaaattgtacatcatatggtgtgctagacatcagcaaatgcaaagaagggagacctgtgtacatttcacttcctcattttctgtatgcaagtcctgatgtttcagaacctattgatggattaaacccaaatgaagaagaacataggacatacttggatattcaacctataactggattcactttacaatttgcaaaacggctgcaggtcaacctattggtcaagccatcagaaaaaattcaagtattaaagaatctgaagaggaactatattgtgcctattctttggcttaatgagactgggaccattggtgatgagaaggcaaacatgttcagaagtcaagtaactggaaaaataaacctccttggcctgatagaaatgatcttactcagtgttggtgtggtgatgtttgttgcttttatgatttcatattgtgcatgcagatcgaaaacaataaaataagtatgtaccaaaaaatattgcttcaataatattagcttatatattacttgttttcactttatcaaagagaagttacatattaggccatatatatttctagacatgtctagccactgatcatttttaaatataggtaaataaacctataaatattatcacgcagatcactaaagtatatctttaattctgggagaaatgagataaaagatgtacttgtgaccattgtaacaatagcacaataaagcactgtgccaaagttgtccaaaaaa

SEQ ID NO: 68 - DNA, 1314 bp, Homo sapiens
aggctcgcggcgggcgctgggcgcgggatccgactctagtcgtaatggaggcgggcggctttctggactcgctcatttacggagcatgcgtggtcttcacccttggcatgttctccgccggcctctcggacctcaggcacatgcgaatgacccggagtgtggacaacgtccagttcctgccctttctcaccacggaagtcaacaacctgggctggctgagttatggggctttgaagggagacgggatcctcatcgtcgtcaacacagtgggtgctgcgcttcagaccctgtatatcttggcatatctgcattactgccctcggaagcgtgttgtgctcctacagactgcaaccctgctaggggtccttctcctgggttatggctacttttggctcctggtacccaaccctgaggcccggcttcagcagttgggcctcttctgcagtgtcttcaccatcagcatgtacctctcaccactggctgacttggctaaggtgattcaaactaaatcaacccaatgtctctcctacccactcaccattgctacccttctcacctctgcctcctggtgcctctatgggtttcgactcagagatccctatatcatggtgtccaactttccaggaatcgtcaccagctttatccgcttctggcttttctggaagtacccccaggagcaagacaggaactactggctcctgcaaacctgaggctgctcatctgaccactgggcaccttagtgccgacctgaaccaaagagacctccttgtttcagctgggcctgctgtccagcttcccaggtgcagtgggttgtgggaacaagagatgactttgaggataaaaggaccaaagaaaaagctttacttagatgattgattggggcctaggagatgaaatcactttttattttttagagatttttttttttaattttggaggttggggtgcaatctttagaatatgccttaaaaggccgggcgcggtggctcacgcctgtaatcccagcactttgggaggccaaggtgggcggatcgcctgaggtcaggagttcaagaccaacctgactaacatggtgaaaccccatctctactaaaaatacaaaattagccaggcatgatggcacatgcctgtaatcccagatacttgggaggctgaggcaggagaattgcttgaacccaggaggtggaggttgcagtgagctgagatcgtgccattgtgatatgaatatgccttatatgctgatatgaatatgccttaaaataaagtgttccccacccctgccataaaaaaaaaaaaaaaaaaaaaaaaaaaaaa

SEQ ID NO: 69 - DNA, 1337 bp, Homo sapiens
gcggcggactcggcttgttgtgttgctgcctgagtgccggagacggtcctgctgctgccgcagtcctgccagctgtccgacgatgtcgtcccacctagtcgagccgccgccgcccctgcacaacaacaacaacaactgcgaggaaaatgagcagtctctgcccccgccggccggcctcaacagttcctgggtggagctacccatgaacagcagcaatggcaatgataatggcaatgggaaaaatggggggctggaacacgtaccatcctcatcctccatccacaatggagacatggagaagattcttttggatgcacaacatgaatcaggacagagtagttccagaggcagttctcactgtgacagcccttcgccacaagaagatgggcagatcatgtttgatgtggaaatgcacaccagcagggaccatagctctcagtcagaagaagaagttgtagaaggagagaaggaagtcgaggctttgaagaaaagtgcggactgggtatcagactggtccagtagacccgaaaacattccacccaaggagttccacttcagacaccctaaacgttctgtgtctttaagcatgaggaaaagtggagccatgaagaaagggggtattttctccgcagaatttctgaaggtgttcattccatctctcttcctttctcatgttttggctttggggctaggcatctatattggaaagcgactgagcacaccctctgccagcacctactgagggaaaggaaaagcccctggaaatgcgtgtgacctgtgaagtggtgtattgtcacagtagcttatttgaacttgagaccattgtaagcatgacccaacctaccaccctgtttttacatatccaattccagtaaccctcaaattcaatattttattcaaactctgttgaggcattttactaaccttataccctttttggcctgaagacattttagaatttcctaacagagtttactgttgtttagaaatttgcaagggcttcttttccgcaaatgccaccagcagattataattttgtcggcaatgctattatctctaattagtgccaccagactagacctgtatcattcatggtataaattttactcttccaacataactaccatctctctcttaaaacgagatcaggttagcaaatgatgtaaaagaagctttattgtctagttgttttttttcccccaagacaaaggcaagtttccctaagtttgagttgatagttattaaaaagaaaacaaaacaaaaaaaaaaggcaaggcacaacaaaaaaatatcctgggcaataaaaaaaatattttaaaccaaaaaaaaaaaaaaaa

SEQ ID NO: 70 - DNA, 664 bp, Homo sapiens
ggattgttggtctgcgtggaacttctcaggtggacaccagagcatggaacacatccacgacagcgatggcagttccagcagcagccaccagagcctcaagagcacagccaaatgggcggcatccctggagaatctgctggaagacccagaaggcgtgaaaagatttagggaatttttaaaaaaggaattcagtgaagaaaatgttttgttttggctagcatgtgaagattttaagaaaatgcaagataagacgcagatgcaggaaaaggcaaaggagatctacatgacctttctgtccagcaaggcctcatcacaggtcaacgtggaggggcagtctcggctcaacgagaagatcctggaagaaccgcaccctctgatgttccagaaactccaggaccagatctttaatctcatgaagtacgacagctacagccgctttcttaagtctgacttgtttttaaaacacaagcgaaccgaggaagaggaagaagatttgcctgatgctcaaactgcagctaaaagagcttccagaatttataacacatgagcccccaaaaagccgggactggcagctttaagaagcaaaggaatttcctctcaggacgtgccgggtttatcattgctttgttatttgtaaggactgaaatgtacaaaacccttcaat

SEQ ID NO: 71 - DNA, 1345 bp, Homo sapiens
aaaacagccggggctccagcgggagaacgataatgcaaagtgctatgttcttggctgttcaacacgactgcagacccatggacaagagcgcaggcagtggccacaagagcgaggagaagcgagaaaagatgaaacggacccttttaaaagattggaagacccgtttgagctacttcttacaaaattcctctactcctgggaagcccaaaaccggcaaaaaaagcaaacagcaagctttcatcaagccttctcctgaggaagcacagctgtggtcagaagcatttgacgagctgctagccagcaaatatggtcttgctgcattcagggcttttttaaagtcggaattctgtgaagaaaatattgaattctggctggcctgtgaagacttcaaaaaaaccaaatcaccccaaaagctgtcctcaaaagcaaggaaaatatatactgacttcatagaaaaggaagctccaaaagagataaacatagattttcaaaccaaaactctgattgcccagaatatacaagaagctacaagtggctgctttacaactgcccagaaaagggtatacagcttgatggagaacaactcttatcctcgtttcttggagtcagaattctaccaggacttgtgtaaaaagccacaaatcaccacagagcctcatgctacatgaaatgtaaaagggagcccagaaatggaggacatttcattctttttcctgaggggaaggactgtgacctgccataaagactgaccttgaattcagcctgggtgttcaggaaacatcactcagaactattgattcaaagttgggtagtgaatcaggaagccagtaactgactaggagaagctggtatcagaacagcttccctcactgtgtacagaacgcaagaagggaataggtggtctgaacgtggtgtctcactctgaaaagcaggaatgtaagatgatgaaagagacaatgtaatactgttggtccaaaagcatttaaaatcaatagatctgggattatgtggccttaggtagctggttgtacatctttccctaaatcgatccatgttaccacatagtagttttagtttaggattcagtaacagtgaagtgtttactatgtgcaagggtattgaagttcttatgaccacagatcatcagtactgttgtctcatgtaatgctaaaactgaaatggtccgtgtttgcattgttaaaaatgatgtgtgaaatagaatgagtgctatggtgttgaaaactgcagtgtccgttatgagtgccaaaaatctgtcttgaaggcagctacactttgaagtggtctttgaatacttttaataaatttattttgataaataatattg

SEQ ID NO: 72 - DNA, 1082 bp, Homo sapiens
agctccctttagcgagtccttcttttcctgactgcagctcttttcattttgccatccttttccagcaccatgatggttctgcaggtttctgcggccccccggacagtggctctgacggcgttactgatggtgctgctcacatctgtggtccagggcagggccactccagagaattaccttttccagggacggcaggaatgctacgcgtttaatgggacacagcgcttcctggagagatacatctacaaccgggaggagttcgcgcgcttcgacagcgacgtgggggagttccgggcggtgacggagctggggcggcctgctgcggagtactggaacagccagaaggacatcctggaggagaagcgggcagtgccggacaggatgtgcagacacaactacgagctgggcgggcccatgaccctgcagcgccgagtccagcctagggtgaatgtttccccctccaagaaggggcccttgcagcaccacaacctgcttgtctgccacgtgacggatttctacccaggcagcattcaagtccgatggttcctgaatggacaggaggaaacagctggggtcgtgtccaccaacctgatccgtaatggagactggaccttccagatcctggtgatgctggaaatgaccccccagcagggagatgtctacacctgccaagtggagcacaccagcctggatagtcctgtcaccgtggagtggaaggcacagtctgattctgcccggagtaagacattgacgggagctgggggcttcgtgctggggctcatcatctgtggagtgggcatcttcatgcacaggaggagcaagaaagttcaacgaggatctgcataaacagggttcctgagctcactgaaaagactattgtgccttaggaaaagcatttgctgtgtttcgttagcatctggctccaggacagaccttcaacttccaaattgatactgctgccaagaagttgctctgaagtcagtttctatcattctgctctttgattcaaagcactgtttctctcactgggcctccaaccatgttcccttcttcttagcaccacaaataatcaaaacccaaca

SEQ ID NO: 73 - DNA, 1487 bp, Homo sapiens
ctagcactctgacctagcagtcaacatgaaggctctcattgttctggggcttgtcctcctttctgttacggtccagggcaaggtctttgaaaggtgtgagttggccagaactctgaaaagattgggaatggatggctacaggggaatcagcctagcaaactggatgtgtttggccaaatgggagagtggttacaacacacgagctacaaactacaatgctggagacagaagcactgattatgggatatttcagatcaatagccgctactggtgtaatgatggcaaaaccccaggagcagttaatgcctgtcatttatcctgcagtgctttgctgcaagataacatcgctgatgctgtagcttgtgcaaagagggttgtccgtgatccacaaggcattagagcatgggtggcatggagaaatcgttgtcaaaacagagatgtccgtcagtatgttcaaggttgtggagtgtaactccagaattttccttcttcagctcattttgtctctctcacattaagggagtaggaattaagtgaaaggtcacactaccattatttccccttcaaacaaataatatttttacagaagcaggagcaaaatatggcctttcttctaagagatataatgttcactaatgtggttattttacattaagcctacaacatttttcagtttgcaaatagaactaatactggtgaaaatttacctaaaaccttggttatcaaatacatctccagtacattccgttcttttttttttttgagacagtctcgctctgtcgcccaggctggagtgcagtggcgcaatctcggctcactgcaacctccacctcccgggttcacgccattctcctgcctcagcctcccgagtagctgggattacgggcgcccgccaccacgcccggctaattttttgtatttttagtagagacagggtttcaccgtgttagccaggatggtctcgatctcctgaccttgtgatccacccacctcggcctcccaaagtgctgggattacaggcgtgagccactgcgcccggccacattcagttcttatcaaagaaataacccagacttaatcttgaatgatacgattatgcccaatattaagtaaaaaatataagaaaaggttatcttaaatagatcttaggcaaaataccagctgatgaaggcatctgatgccttcatctgttcagtcatctccaaaaacagtaaaaataaccactttttgttgggcaatatgaaatttttaaaggagtagaataccaaatgatagaaacagactgcctgaattgagaattttgatttcttaaagtgtgtttctttctaaattgctgttccttaatttgattaatttaattcatgtattatgattaaatctgaggcagatgagcttacaagtattgaaataattactaattaatcacaaatgtgaagttatgcatgatgtaaaaaatacaaacattctaattaaaggctttgcaacac

SEQ ID NO: 74 - DNA, 1543 bp, Homo sapiens
ggagtggccattcgacgacagtgtggtgtaaaggaattcattagccatggatgtattcatgaaaggactttcaaaggccaaggagggagttgtggctgctgctgagaaaaccaaacagggtgtggcagaagcagcaggaaagacaaaagagggtgttctctatgtaggctccaaaaccaaggagggagtggtgcatggtgtggcaacagtggctgagaagaccaaagagcaagtgacaaatgttggaggagcagtggtgacgggtgtgacagcagtagcccagaagacagtggagggagcagggagcattgcagcagccactggctttgtcaaaaaggaccagttgggcaagaatgaagaaggagccccacaggaaggaattctggaagatatgcctgtggatcctgacaatgaggcttatgaaatgccttctgaggaagggtatcaagactacgaacctgaagcctaagaaatatctttgctcccagtttcttgagatctgctgacagatgttccatcctgtacaagtgctcagttccaatgtgcccagtcatgacatttctcaaagtttttacagtgtatctcgaagtcttccatcagcagtgattgaagtatctgtacctgcccccactcagcatttcggtgcttccctttcactgaagtgaatacatggtagcagggtctttgtgtgctgtggattttgtggcttcaatctacgatgttaaaacaaattaaaaacacctaagtgactaccacttatttctaaatcctcactatttttttgttgctgttgttcagaagttgttagtgatttgctatcatatattataagatttttaggtgtcttttaatgatactgtctaagaataatgacgtattgtgaaatttgttaatatatataatacttaaaaatatgtgagcatgaaactatgcacctataaatactaaatatgaaattttaccattttgcgatgtgttttattcacttgtgtttgtatataaatggtgagaattaaaataaaacgttatctcattgcaaaaatattttatttttatcccatctcactttaataataaaaatcatgcttataagcaacatgaattaagaactgacacaaaggacaaaaatataaagttattaatagccatttgaagaaggaggaattttagaagaggtagagaaaatggaacattaaccctacactcggaattccctgaagcaacactgccagaagtgtgttttggtatgcactggttccttaagtggctgtgattaattattgaaagtggggtgttgaagaccccaactactattgtagagtggtctatttctcccttcaatcctgtcaatgtttgctttatgtattttggggaactgttgtttgatgtgtatgtgtttataattgttatacatttttaattgagccttttattaacatatattgttatttttgtctcgaaataattttttagttaaaatctattttgtctgatattggtgtgaatgctgtacctttctgacaataaataatattcgaccatg

SEQ ID NO: 75 - DNA, 1096 bp, Homo sapiens
gaattcattagccatggatgtattcatgaaaggactttcaaaggccaaggagggagttgtggctgctgctgagaaaaccaaacagggtgtggcagaagcagcaggaaagacaaaagagggtgttctctatgtaggctccaaaaccaaggagggagtggtgcatggtgtggcaacagtggctgagaagaccaaagagcaagtgacaaatgttggaggagcagtggtgacgggtgtgacagcagtagcccagaagacagtggagggagcagggagcattgcagcagccactggctttgtcaaaaaggaccagttgggcaaggaagggtatcaagactacgaacctgaagcctaagaaatatctttgctcccagtttcttgagatctgctgacagatgttccatcctgtacaagtgctcagttccaatgtgcccagtcatgacatttctcaaagtttttacagtgtatctcgaagtcttccatcagcagtgattgaagtatctgtacctgcccccactcagcatttcggtgcttccctttcactgaagtgaatacatggtagcagggtctttgtgtgctgtggattttgtggcttcaatctacgatgttaaaacaaattaaaaacacctaagtgactaccacttatttctaaatcctcactatttttttgttgctgttgttcagaagttgttagtgatttgctatcatatattataagatttttaggtgtcttttaatgatactgtctaagaataatgacgtattgtgaaatttgttaatatatataatacttaaaaatatgtgagcatgaaactatgcacctataaatactaaatatgaaattttaccattttgcgatgtgttttattcacttgtgtttgtatataaatggtgagaattaaaataaaacgttatctcattgcaaaaatattttatttttatcccatctcactttaataataaaaatcatgcttataagcaacatgaattaagaactgacacaaaggacaaaaatataaagttattaatagccatttgaagaaggaggaattttagaagaggtagagaaaatggaacattaaccctacactcggaattc

SEQ ID NO: 76 - DNA, 2691 bp, Homo sapiens
gcttgcccgtcggtcgctagctcgctcggtgcgcgtcgtcccgctccatggcgctcttcgtgcggctgctggctctcgccctggctctggccctgggccccgccgcgaccctggcgggtcccgccaagtcgccctaccagctggtgctgcagcacagcaggctccggggccgccagcacggccccaacgtgtgtgctgtgcagaaggttattggcactaataggaagtacttcaccaactgcaagcagtggtaccaaaggaaaatctgtggcaaatcaacagtcatcagctacgagtgctgtcctggatatgaaaaggtccctggggagaagggctgtccagcagccctaccactctcaaacctttacgagaccctgggagtcgttggatccaccaccactcagctgtacacggaccgcacggagaagctgaggcctgagatggaggggcccggcagcttcaccatcttcgcccctagcaacgaggcctgggcctccttgccagctgaagtgctggactccctggtcagcaatgtcaacattgagctgctcaatgccctccgctaccatatggtgggcaggcgagtcctgactgatgagctgaaacacggcatgaccctcacctctatgtaccagaattccaacatccagatccaccactatcctaatgggattgtaactgtgaactgtgcccggctcctgaaagccgaccaccatgcaaccaacggggtggtgcacctcatcgataaggtcatctccaccatcaccaacaacatccagcagatcattgagatcgaggacacctttgagacccttcgggctgctgtggctgcatcagggctcaacacgatgcttgaaggtaacggccagtacacgcttttggccccgaccaatgaggccttcgagaagatccctagtgagactttgaaccgtatcctgggcgacccagaagccctgagagacctgctgaacaaccacatcttgaagtcagctatgtgtgctgaagccatcgttgcggggctgtctgtagagaccctggagggcacgacactggaggtgggctgcagcggggacatgctcactatcaacgggaaggcgatcatctccaataaagacatcctagccaccaacggggtgatccactacattgatgagctactcatcccagactcagccaagacactatttgaattggctgcagagtctgatgtgtccacagccattgaccttttcagacaagccggcctcggcaatcatctctctggaagtgagcggttgaccctcctggctcccctgaattctgtattcaaagatggaacccctccaattgatgcccatacaaggaatttgcttcggaaccacataattaaagaccagctggcctctaagtatctgtaccatggacagaccctggaaactctgggcggcaaaaaactgagagtttttgtttatcgtaatagcctctgcattgagaacagctgcatcgcggcccacgacaagagggggaggtacgggaccctgttcacgatggaccgggtgctgacccccccaatggggactgtcatggatgtcctgaagggagacaatcgctttagcatgctggtagctgccatccagtctgcaggactgacggagaccctcaaccgggaaggagtctacacagtctttgctcccacaaatgaagccttccgagccctgccaccaagagaacggagcagactcttgggagatgccaaggaacttgccaacatcctgaaataccacattggtgatgaaatcctggttagcggaggcatcggggccctggtgcggctaaagtctctccaaggtgacaagctggaagtcagcttgaaaaacaatgtggtgagtgtcaacaaggagcctgttgccgagcctgacatcatggccacaaatggcgtggtccatgtcatcaccaatgttctgcagcctccagccaacagacctcaggaaagaggggatgaacttgcagactctgcgcttgagatcttcaaacaagcatcagcgttttccagggcttcccagaggtctgtgcgactagcccctgtctatcaaaagttattagagaggatgaagcattagcttgaagcactacaggaggaatgcaccacggcagctctccgccaatttctctcagatttccacagagactgtttgaatgttttcaaaaccaagtatcacactttaatgtacatgggccgcaccataatgagatgtgagccttgtgcatgtgggggaggagggagagagatgtactttttaaatcatgttccccctaaacatggctgttaacccactgcatgcagaaacttggatgtcactgcctgacattcacttccagagaggacctatcccaaatgtggaattgactgcctatgccaagtccctggaaaaggagcttcagtattgtggggctcataaaacatgaatcaagcaatccagcctcatgggaagtcctggcacagtttttgtaaagcccttgcacagctggagaaatggcatcattataagctatgagttgaaatgttctgtcaaatgtgtctcacatctacacgtggcttggaggcttttatggggccctgtccaggtagaaaagaaatggtatgtagagcttagatttccctattgtgacagagccatggtgtgtttgtaataataaaaccaaagaaacata

SEQ ID NO: 77 - DNA, 584 bp, Homo sapiens
acactcgcttctggaacgtctgaggttatcaataagctcctagtccagacgccatgggtcatttcacagaggaggacaaggctactatcacaagcctgtggggcaaggtgaatgtggaagatgctggaggagaaaccctgggaaggctcctggttgtctacccatggacccagaggttctttgacagctttggcaacctgtcctctgcctctgccatcatgggcaaccccaaagtcaaggcacatggcaagaaggtgctgacttccttgggagatgccataaagcacctggatgatctcaagggcacctttgcccagctgagtgaactgcactgtgacaagctgcatgtggatcctgagaacttcaagctcctgggaaatgtgctggtgaccgttttggcaatccatttcggcaaagaattcacccctgaggtgcaggcttcctggcagaagatggtgactggagtggccagtgccctgtcctccagataccactgagctcactgcccatgatgcagagctttcaaggataggctttattctgcaagcaatacaaataataaatctattctgctaagagatcac

SEQ ID NO: 78 - DNA, 2179 bp, Homo sapiens
ggcacgagggtcatggacctcctgcacaagaacatgaaacacctgtggttcttcctcctcctggtggcagctcccagatgggtcctgtcccaggtgcagctacagcagtggggcgcaggactgttgaagccttcggagaccctgtccctcacctgcggtgtttatggtgggtccttcagtggttactattggagctggattcgccagcccccagggaaggggctggagtggattggggaaatcaatcatagtggaagcaccaactacaacccgtccctcaagagtcgagtcaccatatcagtagacacgtccaagaagcagctctccctgaagttgagctctgtgaacgccgcggacacggctgtgtattactgtgcgagagttattactagggcgagtcctggcacagacgggaggtacggtatggacgtctggggccaagggaccacggtcaccgtctcctcagggagtgcatccgccccaacccttttccccctcgtctcctgtgagaattccccgtcggatacgagcagcgtggccgttggctgcctcgcacaggacttccttcccgactccatcactttctcctggaaatacaagaacaactctgacatcagcagcacccggggcttcccatcagtcctgagagggggcaagtacgcagccacctcacaggtgctgctgccttccaaggacgtcatgcagggcacagacgaacacgtggtgtgcaaagtccagcaccccaacggcaacaaagaaaagaacgtgcctcttccagtgattgccgagctgcctcccaaagtgagcgtcttcgtcccaccccgcgacggcttcttcggcaacccccgcaagtccaagctcatctgccaggccacgggtttcagtccccggcagattcaggtgtcctggctgcgcgaggggaagcaggtggggtctggcgtcaccacggaccaggtgcaggctgaggccaaagagtctgggcccacgacctacaaggtgaccagcacactgaccatcaaagagagcgactggctcagccagagcatgttcacctgccgcgtggatcacaggggcctgaccttccagcagaatgcgtcctccatgtgtgtccccgatcaagacacagccatccgggtcttcgccatccccccatcctttgccagcatcttcctcaccaagtccaccaagttgacctgcctggtcacagacctgaccacctatgacagcgtgaccatctcctggacccgccagaatggcgaagctgtgaaaacccacaccaacatctccgagagccaccccaatgccactttcagcgccgtgggtgaggccagcatctgcgaggatgactggaattccggggagaggttcacgtgcaccgtgacccacacagacctgccctcgccactgaagcagaccatctcccggcccaagggggtggccctgcacaggcccgatgtctacttgctgccaccagcccgggagcagctgaacctgcgggagtcggccaccatcacgtgcctggtgacgggcttctctcccgcggacgtcttcgtgcagtggatgcagagggggcagcccttgtccccggagaagtatgtgaccagcgccccaatgcctgagccccaggccccaggccggtacttcgcccacagcatcctgaccgtgtccgaagaggaatggaacacgggggagacctacacctgcgtggtggcccatgaggccctgcccaacagggtcaccgagaggaccgtggacaagtccaccgagggggaggtgagcgccgacgaggagggctttgagaacctgtgggccaccgcctccaccttcatcgtcctcttcctcctgagcctcttctacagtaccaccgtcaccttgttcaaggtgaaatgatcccaacagaagaacatcggagaccagagagaggaactcaaaggggcgcagcctccgggtctggggtcctggcctgcgtggcctgttggcacgtgtttctcttccccgcccggcctccagttgtgtgctctcacacaggcttccttctcgaccggcaggggctggctggcttgcaggccacgaggtgggctctaccccacactgctttgctgtgtatacgcttgttgccctgaaataaatatgcacattttatccatgaaaaaaaaaaaaaaaaaaaaaaaaaaaaaaa

SEQ ID NO: 79 - DNA, 3558 bp, Homo sapiens
cagaagccgaaagaactgttcacatggagctgtttattttccggcctgaggttgccgagacaattggcgagctgtcttgaatatatctctatcaattaaaacagcagctgagataaataatgcacctttgccggaactgccacagggactgcaggctcaggcttctcaagccagctcaccgtccagctgagcgagatgtcagcccaaggaaggaacttagatgccttggaaattgatgcctcacagttattttctccagaggaggtgcagggtctgggctagggaaacggaaaggactctgttgcatttaataaagcctgtatcctatggcagcagccactaaggagctcaccagaataagccaatgccattcctcatttggcctgagcagctcagagtcaggaagtcagagcgcagaaaatccagcagctgtcagagggctccatgtttggccacggtctgaagcacctgttccacagccgccgtcggtctcgggaaagggagcaccagacgtctcaggattcccagcagcatcagcagcagcagggtatgtccgaccatgactccccagatgagaaggagcgctctccggagatgcatcgcgtctcctacgccatgtccctgcacgacctgcccgcccggcccaccgccttcaaccgcgtgctgcagcagatccgctcccggccctccatcaagcggggcgccagcctgcacagcagcagtgggggcggcagcagcgggagcagcagccggcgcaccaagagtagctccctggagccccagcgtggcagccctcacctgctgcgcaaggccccccaggacagcagcctggccgccatcctgcaccagcaccagtgccgtccccgctcttcctccaccaccgacactgctctgctgctggccgacggcagcaacgtgtacctcctggctgaggaggccgaaggcatcggggacaaggtcgataagggagacctggtggccctgagcctccccgccggccatggtgacaccgacggccccatcagcctggacgtgcccgatggggcaccggacccccagcggaccaaggccgccattgaccacctgcaccagaagatcctgaagatcaccgagcagatcaagattgagcaggaggctcgcgacgacaatgtggcggagtatctgaaactggccaacaacgcggacaagcagcaggtgtcacgcatcaagcaagtgttcgagaagaagaaccagaagtcagcccagaccatcgcccagctgcacaagaagctggagcactaccgccggcgcctgaaggagattgagcagaacgggccctcgcggcagcccaaggacgtgctgcgggacatgcagcaggggctgaaggacgtgggcgccaacgtgcgcgcaggcatcagcggctttgggggtggcgtggtggagggcgtcaagggcagcctctctggcctctcacaggccacccacaccgccgtggtgtccaagccccgggagtttgccagcctcatccgcaacaagtttggcagtgctgacaacatcgcccacctgaaggaccccctggaagatgggccccctgaggaggcagcccgggcactgagcggcagtgccacactcgtctccagccccaagtatggcagcgatgatgagtgctccagcgccagcgccagctcagccggggcaggcagcaactctggggctgggcctggtggggcgctggggagccctaagtccaatgcactgtatggtgctcctggaaacctggatgctctgctggaagagctacgggagatcaaggagggacagtctcacctggaggactccatggaagacctgaagactcagctgcagagggactacacctacatgacccagtgcctgcaggaggagcgctacaggtatgagcggctggaggagcagctcaacgacctgactgagcttcatcagaacgagatgacgaacctgaagcaggagctggccagcatggaggagaaggtggcctaccagtcctatgagagggcacgggacatccaggaggccgtggagtcctgcctgacccgggtcaccaagctggagctgcagcagcaacagcagcaggtggtacagctggagggcgtggagaatgccaacgcgcgggcgctgctgggcaagttcatcaacgtgatcctggcgctcatggccgtgctgctggtgttcgtgtccaccatcgccaacttcatcacgcccctcatgaagacacgcctgcgcatcaccagcaccaccctcctggtcctcgtcctgttcctcctctggaagcactgggactccctcacctacctcctggagcacgtgttgctgcccagctgagtggccagccacaccaaccctgtgctctctggcccccagctggccacacttctccaggagggacccttggacttctttgtgtgtccagtttggcctcctgcccaaactgtccattccagcagctcctgcccccttctctgtacttgcttctgtctgacaccttctccctgttggcctgaagggagcttagaatgcagccctacctggagatagtgcgggcacctgtggccaagtggagcagaggtggacatggggttggattgttttgattatttatagttacacaaggacttctcccagctgaccctcaggatgccccaagtcaggaagaccattaagaataggaggagagggctctgcctcaactttcctaggaaagagcccacctcggagatagctacggtttcctctggtggagatggtgaggatgaaggctggagagtgagggaggaggctctgctggccgcagagaacacagggatgggagggtccctagccttcgggcacctccagggccagagagcaggctcagagcagctagtgtggagctcagcatccccaccccacccctcctccctgtagagctgatttgaggcctccttctggggctgggctctgcaggccaggtgggtgtggcctgtgttttcccttctgttctttctgcctgtactggatctgttattttcagggaaacaggccccagggcccccctgagcctcaccctaagcccttaggcctctgagagtgctgttgggttctatttatttatttatttgttcctttgttccctacccgtgcccccagtgtcttccctgctgagtaccaggagaggtcctgccccatcctctctctgaagccagggcccttccattccatttagcctttggatcatcctggctgggagaagtgggaccgagccacccagccccactatccccaagcagccctacagccgggatgggaggcacgtggcctctcttttatccgtctatttattttgtaagtgtattcgtgtggaggaggttgttgctttatttttttaaggctctggagtgttgtgtatggtttcttttcacatcccagcctcccatgggcacttctaagaagagaggggatttcttggaaaaggagagaggaatcccctagagcagggaaagcagtgcctgccagctgttgtgcaccttcctgagaaataaatatcctctaaattttcaaacc

SEQ ID NO: 80 - DNA, 39455 bp, Homo sapiens
cgataggatgactcaaagggacaatgccaaatacagtgacggaaagggggaactagaagggccacacattatgtttgggaatataaagtggtaccacaagttggagaactgacactgaatatataatcccttttaatccagcccttccactcagaaatgtgtacagatgtgcacagaaagaaatgtgcaataacacttggccgggcgcagtggctcaagcctgtaatcccagcactttgggaggctgaggcaggcagatcacaaggtcaggagtttgagaccagcctggccaatatggtgaaaccctgtctctactaaaaatacaaaaattagctgggcgtggtggcggacgcctatagtcccagctactagggaggctgaggcagaagaatcgcttgaacccgggaggtgaaggttgcagtgagccgagatcatgccactgcactccagcctgggtgacagggtgagactacatctcaaaaataaataaataaataaataaataaataaataaataaataaaataacactcatagcattattagtgatagccccaaactgggaatattctaaatacaaatcaagagtaatttgaataaataaaatgaggtaggtgcatacaattaaatactatggatgaatgaaaatataaaagctgctactacatccatgaatgtgggtgtatcttactagcataataatgcgcaaaagacgttagaaataaaaagctcactatccatgattcctttttatatagttcaaaaaccgccatcactaaatcaatgttactgaaagtgagatttaaatttgcattggagaagagtggggctaatgtttgggaggagacagaaggtgcttctaggagaccgggagtgttctgctttggtacggttgttatacagtgtgttcaatctctgaaaaatttattaaaacctgcattataatttgtgagtgcatatacacatgttgagatttgtgaatatacatgtatgggtaagttttatcttatcaaaagtttattttaaaaaagttatgaagcataatgttatttgcaccaatcaatgcatcctaacttctttccttatctaatcaaattatatttaattataatctgtattcattttcacattccatctgtgaaaccagggcaccaaatgtaaggaagcccagggtttacaaggttaccacactcttagtgtcatcaggaacacatgagtcactataatctcttttatttttttgtcctggaaagcatcaaaattctaagctactcaaaatgtattgcattttaatgatggttcctatttaccctaaatgtacgaatccaattaagtcaatatttgtagaatcagaacaatttgcttcaatgtgtttttcacttttatttattcactgaagacactggtaattttacactataaaaagtgaaataaaaacatacacaaaattatacttgctatatccttcagtaaagatgagatgactaaaacccagatagatttgttgataggaattattcaagatcatccagctagttgaagagcatcacttagaattctggtgacccctttttaggacaaagctgttcctaaataattctaaagatgtgccagtaacttgctaagaacattgaagtacaagtttttgtgtagatatatgttttcctttttcttgggtccacacttaagagcttcctggatcatgtggtaactctatgtttaaccacttgaattgcagactgttttccaaacctgctgcaccatttttcatttccaccagcagtgcaaagattctattttattgccaacctatgcaatgagaagaaaaacctctgagtgaggaggtatttagaagaactagaatatatccagatgtaagaaaataaatccaaggtagcttagagatgcccattaaatagtttttaaatttttcctagtcttcccaaacctggttacatgtttttactacctggtggatggcactcactcgcaatggtgtttagagttgggaatggactcaggaagtggaaaagttccttcagacaaggaagaactggttcaagacacaaactaaggagtgctaatcggaatgaaagacggggatctgaggaaagtgaagtgaaaatttcctttaggaaggaggtaacatttaagcagaatgccttgttctttaggtagtgtgtctgtccttaggatcttgtgttctggactagtgcctgacataaaaggactgagcactgacatctctttctctcactaattaactttttgtgtcagttgttgtaattccttatatagagtagaatgatctcgaaaggttagatgttttatttaaaaaaaattaataaatgaccaccgtgagtgaatcctaaacaagatagaatgggaataaactgaaagaacaaaatataaacgtatatgtcatatttgctttttgttatgcctatataaatctataattttaaattttgaagtcaaggaaaatactggttattaaattttatcatctattaaaccagtatgatggtaaaacttgttattgcccttcaattatgattcctaattttgcatgagtaatattgtcgttgttatagtcagattattacaattaaattgcgttgcattatatgccttatatttgaggaatttttcctatggaatgactttgcatttatcaacacatttttaacttaggtagattaacttataggttttgttgatttttatcctcaccaacattcttttacaatcacaaaccacagcttcctcttcttgagcaaccgactttacttcatctctttatcagctgtaatacatttttcaagggtttctagtttcataaatccttatgcatatcataatttacttgtttcaaattaaaaattttcttcatattttatttccctagttcaatagaaaatgcatgcagtataatttcttttataaaaactttgcacattttcaaatataattacattgattactgggagttcattttgcaggccaggactctgaagcaagcctgacatttatctttgaaaaaaataacccttacattctttgaatttgtattttattatgaaatatatgtgttttctcattttataaatgtttgaatacaattgtgtgactccattgaatttacactcattagtagttaacagacatggaaattttatttcagattacatttcttcttactggttcttttctaaggactcatttcttccttaggaaaatgtttaattctcaggtttaactttctactctgtttttctgtctgagctctctctttattatctaatgtcatgattctctcctttgaaaaacaaaagtgctactctagtttgccttccatatcactgttttgatcaattgcagtgccaattctgctatattgtcttgaatattgggttttgtttttaatgatgcagtttgttttatttttcttatattgcagcagagtttaaggaactatgcttacattttcgataattacatattttgtgctatttttcatcctaggttatatatttttctttattttattgattatgcaaaacataatgtagaaatgttctggagtccacaagagtgtttttttttttacttaacttttctctttattttttttacaacatcttcttttcctcctttcaattcctccttcctccctttcatttttctttttctattatcttttttaatgggcctcaactttattaactgattgcaaggaataataatcaatgatggttaataacacaattataatgttggtccataatgcacttttattattagtccattatggttcttatttatttattcatatttttagcactcactaattcattcattaatattagtaatataataaattcatgttactatcctgcaaaacaaccacttaagatatcaacatatccagtttgaggttctccacaatctcttaaacatattatttcccaccaccatcaagttaatcaaaattttcaattcaatattctttatcaatgtagtttatttcttctacatgtattcctttaaaaagctgtttatttcttttaaacattataaaaaggatgtcatactagtgaagtctaatttattaatttctttctttatgctagatattttgtttattttctctaagaatttttttttatctctagggtcatgaaatatgcttctatacccttttgtagaggatttactcttgggcctttcatatttatatttacaatttattgatgattaatatttgtatatggaatagaattaagattcattttcatataatacagatactgaattgatccagtatgattaatttattttacttctactactttgaagtagcacttttattgtaaatcaaatgactacgcatgggtggagcagtttctggattctcaaactgattgaactggctaatttgtttgacacttcactgataccatatattttaattcctgtaacttacaggctttggtattgtgtagtattagtcctccaacattttttatcttagcaagactgtcttggttactttttgcattttgaatgttcatatatatttaagtaatgtcttttcaattgcaacaataattctctgagattttttattgtgaatgttttcaacaaatttagggagaatatacactattaagtctcccaattcatgagcatggtgcaaccttccatttattggagttttctttatttttatccaactgcattttgtacatttctgtttggttttgttgaacatattttatgtgactttttatttgggcatattgttaaagaaaaattgccaaagtaatataagaactccaatgtatacgttacccaaattcatttagtaaccatagatgactttctacttccaaattctttctatatttatgagttggcatctagttactactgattcagaacaaatcacccaaaacttaatgacacattacaattgacatcattatactattatctttgtagttgttaggtgtttcctgggctgaccaagatttctgcttgggatttcttacatggatgtagtcagatagcagctggggatggagtcatataaaaggtggccaattcaggctataggatgagtcctcagctgaggctgtgaatctctacatgctcctgcttggcttcttgtacacttcctcgaagagtaccagacagatgttttataacctcttatgacttactatagcctcagaagacacatagtgttacttctatcacaattataggttcactaagattccaaagggggaaaagtatgctaatatgtccaatagggaaattatcaacatcacactattagaggaactaataagatggaagatcttgtgactatcttggagtatccagttggcaactctctacgcttgtttaaatcaatctacatttttactgtatgcaacatatactaattttcatctgcaacatctacaagtatttcccatgatggtggtaagttaaagttcaagatctcctcatctagatcagactctgtgcagttgagcctctttgcccatagttcctaaatagcacctgtccccctatcccactcaagatttgtgaacaatgatgagacaggactaggatgcacatacttgacagacaatgctgtagatactccctttcaggaagaaggcactcagcagtcaaaattccacagagcataaagccacagcttcctttcagggcttcctgcttcaaatgtctgtgttttttaaattttttttccctcaaactgtacttttcttttttatttttttgccttggaaataatgtaattattatttaaaactcagtgaaatcatgaggatacagtcaggcaaaccctaaatgtgggaaatcctataggataaattatttctttcttttttgttttttaagtgtgtaattcttttttttattatactttaagatttggggtacatgtgcacaacgtgcaggtttgttacatatgtatacatgtgccatgttggtgtgctgcactcattaacttgccgtttagcattaggtatatctcctaatgctatccctcccccctcctcccaccccacaacaggccccggtgtgtgatgttccccttcttgtgtccatgtgttctcattgttcaattcccacctatgagtgagaacatgcagtgtttggttttttgtccttgtgatagtttgctgagaatgatagtttccagcttcatccatgtccctacaaaggacatgaactcatccttttttatggctgcacagtattccatggtgtatatgtgccacattttcttaatccagtctatcattgttggacatttgggttggttccaagtctttgctattgtgaatagtgccacaataaacatacgtgtgtatgcgtctttatagcagcatgatttatattcctttgggtatatacccagtaatgggatggcagggtcaaatggtatttctagttctagatccctgaggaatcaccacactgatttccacaattgttgaattagtttacagtcccaccaacagtgtaaaagtgtttctatttctccacatcctctccagcacctgttgtctcctgactttttaatgattgtcattctaactggtgtgagatgctgtctcattgtggttttgatttgcatttctctgatggccagtgatgatgagcattttttcatgtgtctgttggctgcataaatgtcttcttttgaggtgtgtctgttcatatcctttgcccactttttgatggggttgtttgtttttttcttttaaatttgtttgagttcattgtagattctggatattagccctttgtcagatgagtaggttgcaaaaattttctcccattctatatgttgcctgttcactctgatggtagtttcttttgctgtgcagaagctccttagtttaattagatcccatttctcaattttggcttttgttgccattgcttttggtgttttagacatgaagtccttgcccatgcctatgtcctgaatgatattgcctaggttttcttctagggttttcatggttttaggtctaacatttaagtctttaatccatcttgaattaatttttgtataaggtgtaagaaagggatccagtttcagctttctacatatggctagccagttttcccagcaccatttattaaatagggaatcctttccccatttgtttttgtcaggtttgtcaaagatctgatggttgtagatatgtggcactatttctgaggtctctgttctgttccattggtttgtatctctgttttggtaccagtaccatgctgttttggttattgtagccttgtagtatagtttgaagtcaggtagtgtgatgcctccagcgttgttcttttggcttaggattgacttggcaatgcgggctcttttttggttccatatgaactttaaagtagttttttctaattctgtgaagaaagtcaatggtagcttgatggggatgacattgaatctataaattaccttgggcagtatggccattttcacaatattgattcttcctacccatgagcatggaatgttcttccatttgtttgtatcctcttttatttcattgagcagtggtttgtagttctccttgaagaggtccttcacatctcttgtaagctggattcctaggtattttattctctttgtagcaattgtgaatgggagttcactcaaactgtactttttatcccttcaagcaacttcatcaaatcaaacaacaaataatgagtttttagcagtgtcttctatgttgatcaaaactctcattatcctttgaggcagtttaatgtaaactttcttcattaattctttgtgttttcactttattatgaattttttttcttgaatttacactgtaaggcatggattttttattttcagttatagtcggtatggcttttgtataaaattctccacattcttcttttgctttgcttccctcaactctaaatccccaaattctgttagtatggtaactgacctcataatcttgatccattttgtatggaacattcccaggttaggttcataccaagaaaatgactctgtattcaagccacttgaattaatagctgtatcagtgattattatttatgatgaccatggtcttataaggttcatataacatgcttgtggtcacttgcattagtcatcatcagaacaagaccagctgcagctgaggactgaggaaatgttgtggtgatttggagtattattaagcgaggggttccacatagtccctctacagactgaagacactggggaaggagcatccgtgtgtgtgtgacagctgtgaaataatctgttctggaacaagaagctccaaaatatcacagcctgggatgactttgtgtgctttccatagagcatttggctacatatcaaagccgttattagtgggctgttccctggctcagggcaggtgtctgcctcagccatgtacataatggacataaggagctcaactcttctgtctcctgctgcctgatcccagatgaggaaaaggattatgaggaggtgccacatgatggtgaaatttgctttcttctcattgtaagttgaatctttagtaccttttttggtctgtgacatttgatttctcatggagcactcacagtgttgagtaacatgataagctcatagagtgggatgtgtttaacctcactgacatttgtgcttatgtgattttttcaaaaaaattcagatgtcaatgagaatattgtgccgcctcagttttatttatttttatttttttaacttttgttttaggttcagggatatatgtgaagttttgttacataactgaacttgtgccatgggggttccttgtacagattactttgtcacccaggtattattcccagtgcccaatagttatcttttctgctcctttcctttcttccaccctccaccctcaggtagaccccagtgtgtattgttcccttatttgtgttcatgagttctcaattttcaagttctggacaaaggttgagggaagcaagccactatccagaaccctagtgtctctgcatggttgagtgaccacgagtctgaggtagattttgctcccacaatcagcagcctgaagcctgaagatgcagggtactgttactgtcaacaacatcaaatcttgcctctctcatgtgacgaaactgagcaaaggcagtgcaatgatccagcagtgttatcttgttcaagttactcatacataattgatgaaatcaggtagaaagctcagtgaaagagattttgaaatattagtttctgtgataacagaacacacagattgtaatcacatatcattggttggaattttgtctcttacacttaatatatgtgtaaatttggcaaatgacttaaacacttttaccttgtttttttatctctaataaaggaaaataaagaagtaactataccataagactattataataattaagtaattgaatacttataaaatgtttataactttcaaatgtattaaacactaaataattactaataatcattataattttgctacatctcttaattatgtagatccagtgtttccccaaatactgttttctttgacgttatttacaaaattatgatttttcccctaaaactcccactatgttaaatagcagataaatttatttcatgccaagctgctaaaaacagatataaaaagctggacaaaatataaaaagctgatactctaaggtaccatgtaccttcgaataagtgctatgtaataagcatctgactccatttttgatgtttgatcagtgacagctttcaatcaccacctcccactttcccttccaccacatatttgtgcaactgcctgcaggacagtcaaacctcatagatcctcagcaatgcaagatagcatatctccagtccaactataaaaactcagccctctgtgtaactcgagccagcttataccagcttgtgcatatcctgctttcccccagattcccttgtgtgagttagaaaatttctcccaaattctcttgtacatggagtgtcaacagcttcaccataatatctactaattagaaaagatccatctcacctccgtgggtgaccacaaaatatgccaagagagcaagtatttgatgaatcaagaaaataaggtaagcttttatgaactgaatatttgtgtcccctcaaaattcaccagttgaagccctaactccatgtgcgagtatatttggaggtagctctaagaaactaacagtcaaatgaggccataaggttgagattctgatctgattcaattagtgtctttattaaaaaaaaaaaaaaaaaggagagattgggctcggtggctcatttctgcaatcccagtactttgggaggtggaggcaggtggatcacgaggtcaagagattgagaccatcctggccaacatggtgaaaccccgtctctactaaaaatagaaaaattagctgggtatggtggcacacgcctgtagttccagctactcaggaggctgaggcaggagaatcacttgaacccaggaggcagaggttgcagtgagccaagattgcaccactgcactccagcctggtgacagagcgaaactccatctcaaaaaaagaaaaaaaaaaaagagaccaaatctattaggccattcttgcagtgctacaaagaaatactgagactggtgatttataaagaaaagagttttactcagctcacatttctgcaggctttgtaggaagcatgatgctggcatctgctcagcttctgggaaggcctcaggaagcttacagttatgatggaaggctaaggggtagtaggcccatcacaaggccagagaaagagcagaagagagagaaggagttgccatatgcttttaaataagcagatctcatgagaactcgctatcatgagaacagcaccaagaagatggtgctaaactgttcatgagaaatctatctccatgatccagtcacctcccatcaggcctgacttgcaatactggggattacaattccacatgatatttgagcagtaacaaatatgcaaacaacatccttttacccctgggctctctcaaatctcatgtccttttcacatttcaaaatacaataattccttttccatatctgcccaaagtcttaccttattgtaattttaacacaaaagtcccaagtccaagtttaaagccacatctgatactcatattcttccactgataagtctctgaaatcaaaacaagttatctactttcacaacaatcaaaagacaaaatcccattgattagtcacagcaggaattaaaaacttagaaaaatatctattttgagaaataagtaccatgttgatatagccacatattcttcaacttagtccctaggatttcagattcttggaaatcatgtctcaactgtgtgcatcctagtatggcaccaatagcatctcaacctcccactttagaagtagctcaatcaattctaaactttttcatttagtttctgaaatattctaagtgatgcgtaggactatatatttgtccaaattactcaggaacatccatccactggtgggtaccactatgttttaatagacaccagtcctctcttccttccttcacgtcatcaacattccagtgttgaatggccatgatggaaatatttgacatttaagagtgagcataatttatttaatcagtattctctattggagagcaggctttaagtagaactgaattctgaaaaaaataaataagtaaaaagagaatcagatagtgtctgagttctttcatgcaactataacaaactcacagactgggaaatttataaacaataaatatttatttctcacagttctggagttcagaactctaggatcaagatgctaacagattcagtgtcggtgaagctgtctggtggagccagaaaaggcaaaggagacaaattgaatcttgcatctgcacatggcaacagagatggaagggccaggcagctctctgaaatcttctatataaggccattaatcccatttattaagggcagagcccatgacttaatcacttcccaaggggttctaccttttaatatcaacttaggctttaaattccaacattaagtttggaacatcacaaacatctaaaccatagcagatgggactagacaattcctaacaaagtcagcacataaccatataggaggagtgacaaaagcagctgccttggttacctttgaccaagactttcttacaaaaagggttccttagcaatattcatttatcaacaccagtgatgacatgttgatactgtgtaactcttgataggatgtactgaagacacatccctgctgtaatattcttgccaaaaatgaaaaatctgactttaatcaatagaaaataccaaacaatagaacttaagggacattctgaaaaataaccagccagcataaatcaaaagtttcaaggtatttcaaaacaaagactaaaaagctgtcagagattgaaggaaattaagaaagcatgaaaactgaatgcaatatgggatccagaaattttatcctaaaacattaaaagtaaaaatggtaaatacatgtatcagtggaaagctcagtgaaattcaaatgtagattgtaacttcgttaataatagtggattaaccattaatgttaaagctatttgaagtactagaaaaatcagtttaaaatgattttatattcagcaaaactatccttaaagaaaagaaaagaagccgtgactagcatatatgtcctataagaaactcaggaagaaatccttcagaattcagaatcacagtaaatgacaatgaacagtaatttaaatccatgaaattaaatgaaagcttcataaatatacttacctcaactcatatgttgttgatgttcacgaaaactgaatctttgtgatagatatcagagttgcagttcccttggtaggttagaggcagaagctattgactagaaaggtgaatgaaggcagcatgtggagaatttcaaatcattcatatttgtatctgggtagtgaatgtgagtactttatttggttgagcagtgaacatgtttgcactttactcagggcacaatttattttgatttataaaattaacagcaaaccaagaccctttcaacacacatgaagaaaaaaataagaagcaccaaatatttacagaaactcagccgtattaaagagaagtgtaacaagcactgggaaaatactaggaagtaaaaaaattgacagtaaacacagtaaacatagaaatatatcctgtcccaatcaggctgcatagattgttatttctgccagttttttctcaagcatacaaaatatgttgttcataggaaaggcccccatacccctgcacatatcatgttatttctataccactgcacccaccaggggatttgcatattgtcccccagggaggaccttcccttgcaagtctgagataaaagctcagcaccaaccttgacttgactaattaggactcctcaggtcaccttctcacaatgaggctccttgctcagcttctggggctgctaatgctctgggtccctggtgaggacagaagagagatgagggaggagaatggggtgggagggtgaactctgggggccccattgcctcccatgtgtgttctgtcctcatgttagatgtgtacgtcttgtactccaggatggggcttgtaacttttatatctgcgtgagtaaggcatgtgaggtttagatctgtaagaatgaggaagattccagaaggaacaaagaccagtgctccggtgaagactctaacagagaaagagggaatggtagaggaaacttctagcactcaaagcactctgctgtgctttgaaaatatgtttttattttgaaattatatattactagggtctgaatcaaattataaaaattgatttagcctgaaataaataacagaagaaaaattattttaaaattgtgcttaaagtttctacataaccttgcacttctctctcattatttcaggatccagtggggatattgtgatgacccagactccactctcctcgcctgtcacccttggacagccggcctccatctccttcaggtctagtcaaagcctcgtacacagtgatggaaacacctacttgagttggcttcagcagaggccaggccagcctccaagactcctaatttataaggtttctaaccggttctctggggtcccagacagattcagtggcagtggggcagggacagatttcacactgaaaatcagcagggtggaagctgaggatgtcggggtttattactgcacgcaagctacacaatttcctcacacagtggtacagccctgaacaaaaacctcccgctggagtggcccagctgctcaagtgtgttgtttctctggggagcagttgaacagaatctctatctgtatgagataaacatgttggagaactcagggcaacaggttgcatctgagggttctgtcccatgggtgcctcagttgtacgtcaggcaaaacctgttcacagccctgtcagctgcaacagccttggcatggcataagccataggaaaccagaggtgatcccagtgcctgcacaggtaatagactgccctgagggagagcttaagaaaatcctattccaatcttccctgccttgcctgcattgggaaataagacttaaagaggtaaataaccagacaagtaacccagatttgttgcaacacttgaatatatcttgaggtttagcagtttaaagtctatatttaggaggataatatgtggtaatatcccaaaattgaacttttcaactttcctaacttcttatttttctctttcaccacctatcttcccaccacatattgatggtggaaagagccttccgcacaagctgtcatcatgaggagctggatgagggcaattagtgaaaatcttggatttcagcctcagaatggacttttgtaaattggtgagagatagaaaatatgaatgctaaaattattttattcgcttcaattgtgtcttgctgacagaaaaggatagtttttgaaatttcagaagttgagtttcataaacagaaacttaaactagaagacataggttatagaatttacctcatagaacactgaaataacacagaatgatgtgcgatttctttccccaaaatgtaagagtttgaagacagtgggccgacttcaagaatgggagaattaatggaagatagtggaggtcaactatggcccaataacctgctctttgacttacattaggtacagttgtggatgacagtgactgttgggggttggtgatataaactcagaaaggagcccaaatgtctttcttatgaagaatcacagaggagaaagtatcactccctggctccatgggttgagcctgcaccactgcaagtttcaaggaaaagtagttcatcaagaatgatcttttagttctgcaatcatcaaatgtttattgaagttcctgtgcaaatagacctgaggttctgtgacttagtcacagtcaaactaaaacaacccagcagatgccatgtggttgggtttgagaacacaaatcatgcagtggcatgctaacctgaagtcccaatagagcctacatcaattggggagcagtggcaatgatgaccaatatatccatgattcagacatgtattatgaatggtctgcgcagaatttatcaacaacaaaaactccatgaatcctctgtatggggagtttctgtctttctagaccagcacccaaagactgcacatgtcatcaaaccacagccaatgttccatggagaacactatctgtgagttgaggctgcattgtgcaaccaaagaggcacagccagattctcctttcacagatgagtttctctgcctgtgccaaagcagaacttgggtccaaatgccaacctggcaaatatggcaggagaacaaaaagtcaggtaagcatcagctcaattagagaggatttcctcaccctggaattttagattacctaggccttattctgtccactgttctctgatgttataatttcataaattttgtattttttgtaccttttgcagcagttgctttagggcttttaaccacaatgttattgtacctgggagtggagataactttttcaactaaataatgttttagaaatgacaattttggtattcaattgtcatgaaaagaataaatggttttcaatatataagtacatgcatcgttttcacacaatgtagtcattacatgaaaatgaacctcattcctaccttctagtagtaattgtatagaaaatatatagcttgcatagatgacacttaaaataatgccctaaaagtatttctaaactaatcatgacatgatatgatcaaagtaaaggggcatttgaatcagcaggacaacatactcttttccttgttaaggaagtaaaccatattagaaatgactgtatattccaagataatgcattctgtggtgagggaagttaaaatccaatttttgaggagagaaatccagaaaaaaatggattatggcaagacgtttgtaacataggcaaagaatgacaatccttcaaagtatttttctgcacatattcaaaagtggagacacacatgcagtcaaaattttaatgattacatactcacaatcacttctgtggggcctggagatactgcacatacgactgttagcaagacactcactgggacgctgcgttgtgtgatggccccacatacaaacctcaaggaggctcagcctctcaatgcagcaggagcagctggggtacccaggccacacgtccataccaggtgggctcagttagagatggctggagagccttccaggaagaggccatgaggtttcagtcacaaacactggctcctcttctgtgtaaacaggggctagagccctccaggacaattcctagagcctctccctttctctccaattagtgcgctgacaccctacagactctccaggaagtggttgtcatgtcctccctgcaacagccactaaagttccctactgctgtcatgaatgcagggacacttagtcacatcactgggaggcgaccctagtgtatcctgacctcacctgctgccactgatgactttcagggcacctctttctccctttgctgagtgactctcactctcaccaaccatcaggagaatggaaagctgcctgcaatgcatgatgttggctgttgagcaaatcaaagctcacaggagtctcaaacatgtacaccacataataatattttctgataatactatttggacttttcttcctttcaattctggaagtaattgagaatattttttgaactcttagaaacacttagtatatatgtgtagtaggtagtaactagttttgtctactggtttattttgtttgcttgtttcaggccatgatgcggcatgttaaaatactgaagacaaagatacattttagaattaagcatactgtacattggctctttccacaccactgcaaccaccaggggatgtgcatattgtcccttaggaatgaacttcccttgtgagtctgggagaaaagctcagctgtaaccttgccttaactgatcaggactcctcagttcaccttctcacagtgaggttccctgctcagctcctggggctgctaatgctttgggttcctggtaaggacagaggagatgagggaggagaatggggtgggagggtgagctctgggggccccactgtcacccatgtgtgttccgtccacatgttagatgcacgtgtcttgtgctccaggataaaatgtatggtggcacttttatatgtgaaagagtgaggaagattccagaaaaagcaaagacctgtgctctggtgcagattctgacatagaaagaggagggtagcataagtgacttccatagggcaacttgggccttcaaaatgtctgtttttttttttaattgaatttttttggtgcatgaatcaaaattacacacacactcacacacacacacacacacacacacgccgcaatacaattatttagcattaaataattgtagagaaattatgataatgtctcatgatttacataacattgtacttcttttttatattactttaggatcctgtgggaatattgtgatgacccagactccactctctctgcccgtcaccaatggagagccggcctccatctcctgcaggtctagtcagaaccttttacatggtaatggatacacctatttgtattagttcctgcagaagccaggccactctccacagctcctgatctgtaggacttccaatcagttttctgccttcccacacaggttctccccaatgggaggagagagtagaccagtcatccccagatatatcacaggactagtttcaacctttggaagctggtctatatcctatggttaaataggcatttgtgatacgacctgaaatacatttggacaagaacttcactaacaattgagtcactgaagacttacggccctgtgtgacgcaccacataaccgtgagtttgcagtggttgcaggtcagggacagattttatgcttaagatcagtagggtggaggctgaggatcttggctattacaactgccaccacactctacaatatcctcccacaatggttcagcaccaaacaaaagcctcctgcttggattgtcccagctgcccaaattagttccttcactgaggagtagacagggtatattctctaaatctatgtaacaggaagatgttggtgaactcaggggattagtatgaagctacacctcaggcatcacacataagatcacttcagcagtcgcagccttagcatgggcagaacctacagaagatgcaagtgccctctgagccaggagacaggaggaaggaggaagggaaaggtgacttagctcatctcaatcctctctcctttgcatacatttgtcaaccagatgtattcagcctaccagtcacacaactgaggctgatacatgacaacatagcactggtatattcttggtattgtttggcttagcagttactagtatatatttaatgggagaatatttggtggtgttaacacattgcttatctcccttaccccagttgtactttacacttgttctcggcacacattctcctccaggactggagcattcacagggttttatgttactgttcttatgggagtaaaaagaaaaacgattcacattcttgctactgagctaggctgggatgtcctgggccaagctgaaaatgtgaaaaataagagtatgaatatttattaagttttatctggatctaagatacttatccatgaaccagtcctgcagctgtgcccagcctgctccattccctgctgatttgcatgttcccagagcacaaccccctgttctgaagacttcttaataggctggtcacaccctgtgcaggagtcagtctcagtcaggacacagcatggacatgagggtccccactcagctccaggggctcctgctgctccggctcccaggtaaggatggagaacactaggaatttactcagccaatgtgctcagtacagcctggcctttcagggaaatcatcttacaaatagttgtgtggattatttgtttttatgtcccaggagtcagatgtgatttccagatgactcagtctccatcctccctgactgcatctgtaggagagagagtcaccatcacttgctgggcgagtcagggcatttgcaattatttaagctagtatcagtagaaactagagaatcctcctaagctcctgatctatgctgcatccagtttgcaatctggggtcccgtcacggttcagtggcagtaggtctgggacacatttcacacattctcaccatcaggagcctgcaacctgaagatgttataacttattactgtctatagacttacagcagccatcctagagtgttacaggtcataaaataaacccccagggaagcagaagtatgactcatggctgccccaggtgcttccactggtgcctccatctgctgagagtgtttctcaggtgcagccaagatttaaaggtttttgtaggaatggtcagaagtctcatctgcattctaattctttttcttcctgcttagccccagcagcacagacatgacactatctctcctgatttaataaaggatagcatttacaatacctgaagaatctgtgttattgcatccatctgggtcatagattaaaagagaaaccactctacagattgccagaaggcattgttttaatacagggaattagagttgaatatacaaaactgggagtgtggtagttagggaagctgacactagaaacacgggagtctctggaggtctgccagaagccagagttcatcagccgctaaaggcatgggctatctaaccatatagtcttctttgtctaggaagtccgtatgcgaagatgctgatgctatcagttgttgcagcacctcaccaggtgattctccagtccttatctcagtgaacatgtttgcctaccggtgtcaaagaatattgaatcgccttcttcttaccttcaaatatgatgagaggtcttctctttgagtaactctacaagaaaccatagagggtttaatgggtttcaggaaaggtgcttttagaaatcatggtgaatatgaggaattacagccaagtgggataagtatttcccaaaatctcagaattttccaggtatggggtggcttcagaatacatttggatgttcttacatgtattattagaaagtttggtattattgcaagaaaattttattaagtcgtaaagtaaaagaaaaaaatgacaacattgcttgaaatacatagcaatcctttgacaaatgaaaaaaaaattgacaaaacaaacaagaacacctataggtgcatgtagcatactttttccttaatataagagcactttgctacttaaaatttgtccagattccagtggcattctcagcgtcactatgaacacagtacaaatgcaaagtagcagatgtgctttagaccttgttgcatgataacctgcacttcaactagttaagaggtaacgtacgggtgtttcaagaagccaagttttagaagacatttactttagctaaagatttttttttcccccacagtgagaccatttatgttaaaaccacttaaaaatatatgctgctttatttctaattaatgcaaaattacattcaaaaatatttttaatattctaaaagttgaaaaacaattattttttatcaatggatcaaatactttgatagttaaatgcagtaaacgtttttagaaactttaggacttaacaaagtaaaagaataaattaaattgtgttcactgttttagagaacattaggataccatttgcctggtcagttttgtttgaaaattgtgttcctttttgctgccttccatacaaatgttgtgtcttggctaggcccttccttgatcccaaatgaaacacaatctaaaggcagaagaaccactccactaagctcttccttgatcagccacatcattgttatcataaacatctattaacaagaaaatatctgcttagttttattatccgctgagttttgagcagtggataagtgcatgtttccgtaagtgcactttttccataagtgaggtgaatttcacttaattcatatcatttagctttaatttcctctaagtgtctttataaatggatgactaaatatttatatttatgctatcagatttgataacatgcatctatctatatgactggatgtgtgaatattatattggtcagctttcacccaggtggtcatgtcagaaaaggctgttagtttagcctgagtgtagaatttctatcttagatcacatatatcatgtgtcttcctgtcttatatccctgtgtcttcctgtctcaccaattatctagattcagtgaatggtgtgtggtacaagacttgtaggaactaaattaagttgtgtggtcccatttcttttgtttctaccctaaatatgcctagttgttttccctggtgcatgacagaatatggttggaatgaagagttattggaactttatctcccaagtacacctttcacttgctgcttagggatcttttctgagggccctgaagcttcctcaaagagcaacactcaagtacccacagtgctgcaggtgcaggggtgaccacaactgcacagatgagaagcacccaggttctgacccttcaggttaccaatgccatttccctgaagacagacaatcatgctgtccatgcaggtaacagacaatgatgctgtccatataggcaggggacaactccttgggtgatcctctaatctacacaccgcttgattctgtgcaatgcttatatcaatccagagtcaggttctcttctccttaatagttcccagaacctctgcttacaccccctgaatctcatttcatatactgctgctcctttcctttaatcagttaaaatcgtttgctttttcttcctttctcttaggtatcaaggaagcagttttactaatgctgctctaagtttcaattggatcttcattcattctggaaatagagtcaacaatatttatctaactgtcaagacgttatcttggcaagccctgaaatcaaatccattgtgttggagacagagctttaatccttatagattatgtgccattagtaaatttgcttatgtgaaactttggcaataatagaatctacctaaaaggtctctttacaatttatacaaggtaaagcatttacaatagtatctaatcattatatgtgctggtattaattttgttgttactattatgataacatttagcactgtaataatcattattatcatcactagactaatttagaagagagttaggagaaacaatcttaattctaatccaaggatgtttcatctatagccacattagtttctgagatgggattttcactgactgactcacaattcttaaaatgctaatgatttgttcttgatctatactaacttgctcagactttcaatcatgcccacccagatgggtccattgcatttcttctcatcattcattatcataactttatcctatgaaaggttagaatgtcatattgctgtcctttcttacataatctttattctgtctttttaaccttttctcattttttctactacatctgccataactcaaaaaccaaatctcaggtttttcccaggattggcatgcttctgtgctaaagatgttgttcattctcttactttctggatttctacgggacaaattatttcaaactcaggcctttctaatacctcagaggtatagggcataaaagagaaagaaaaagcatatgtatgagtgtgatttgacaaattgaaaagtcacttcacctttttgtgaagtcatctattctttcttgcaagggttttcaagttgtgcctatatttttaaacacgtatgacttcttcaaacacttttcttctctaaatcttttcctccaaaagccccagtcagattaactgtatccagtaaagtatggttgacccttctctgatatcctctctatatatacccaaaagtttccattctcttctaacatttttgtttcattaccatccaaagacaaaattctattaaattttcagataataacttaaaaatttggagaagtacatatttctagaaataactgtcatgcatatgtagccacatgttctttaactgagggaccagaacctcttatttccacaaagagtgtctgaactgtgtgcatactaaaatggtacaaatggtatctcagtctcctcagcagaagtagctcagggcaagctgttcctatccatttgattcttgcagtattccaagtgctagaaaattatgtttttccaaacagttgattcagtaactgctgttcatttgttggtaccactacattttaataaatctcattcctctgggttttttttcaggctattaacatttaaatggtaaatggccatcatagtaacatttgccatttaaaagccaactcatttatttgttcaatattctctattgtacagtaagtgtgaagagggttaaagcctaagaaacataaaaaaaaatagtttcagacaggaataggttatttctcagaaagtcagcaaataaccaaatacaaagagtgatagaagcagctggcttaattagctttgtccaagacctcctttcagaaaccagaatctttgggacacagcaaaagcagtgtttaaagggaaatttatagcactaaatgctcacgggagaaagcaggaaacatctaaaatcgacacccttacatcacaattaaaataactggagaagcaagagcaaacaaattcaaaagctagcagaagacaagaaataactaagatcagagcagaactgaaggagatagagacacgaaaaactcttcaaaaaaaatcaatgaatccaggagctgttttttttgaaaagagcaacaaaatagataaaccactagccagactaataaagaagaaaagagagaagaatgaaataaacacataaaaaatgataaaggaggtatcaccactgatcccacagaaatacaaactaccatcagagaatactataaacacctctaaacaaataaactagaaaatctagaataaatggataaattcctcgacacatacaccctcccaagtctaaaccaggaaaaatttgaatccctgagtagaccaacaacaaagtctgaaattgaggcagtaattaatagcctaccaaccaaaaaaaagtccagggccagatggattcacagccgaattctaccggtagaaaaagaagctggtaccattccttctgaaaatattccacacaatagaaaaagaaagaatactccctaacttgttttatgaggccagcatcaccctgataacaaaacctggcaaagacacacacaaaaaagaaaatttcaggccaatattcatgataaacattgatgcaaaaatcctctataaaatactggcaaaccgaatccagcagcacatcaaaaagcttatccacccatgatcaagttggcttcatccctgggatgcaaggctggcttaacatatgcaaatcaataaatgtaatccatcacacaaacagaaccaatgacaaaaaccacatgattatctcaatagatgcagaaagggtctttgataaaattcaatacctcttcatgctaaaaactctcaataatctaggtattgatggaatgtatctcaaaataataagagctattcatgacaaacccacggccaagatcatattgaatgggcaaaactggacatattcttgtcaaataccggcacaagacaaggatgccctctctcaccactcctattcaatatagtattggaagttctgggaagggcaatcaggcaagagaaggaaataaagcatattcaaataggaagagaggaagtcaaattgtctctttttgcagattacatgattgtatacttagaaaaccccatggtctcagccccaaatctccttaagctgataagcaacttcagcaaagtctcaggatacaagatcaatgtgcaaaaatcacaagcattcctatatatcaataatagacaaacagagagccaaatcatgcatgaactcccattcacaattgctacaaagagaataaaaaacttaggaatacagcttacaagggatgtgaaggatctcttcaaggagaactacaaaccactgctcaaggaaataagagaggacagaaacaaatggaaaaacattccatgctcatggataagaagaatcaatatcgtgaaaatggccatactgcacaaggtaatttatagattcaatgccacccccatcaagctaccattgactttcttcacagaattagaaaaaactactttaaatttcatatggaactaaaaaagagcccacatagccaagacaatctagacagaaagaacaaagctggaggcatcacgctacctgacttcaaactatattacaaggctacagtaaccaaaacagcatggtactggtaccaaaacagatatatagacaaatggaacagaacagaggcctcagacagatgctggagaggatgtggagaaataggaatgcttttacactgttggtgggagtgtaaattagtccaaccattgtggaagacagtgtggcgattcctcaaggatctagaaccggaaataccatttgacccagcaatcccattactaggtatatagccaaaggattataaatcattctactataaagatgcatgcacacatatgtttattgcggcactgtttacaatagcaatgacttggaaccaacccaaatgcccatcaatgagagactggataaagaaaatgtggcacatatacaccatggaatactatgcagccataaaaaggatgagtttatgtcttttgtagggacatggatgaagctggaagccatcattctcagcaaactaacacaagaacgcagaaccaaacaccgcgtgttctcattcataagtgggagttgatcagtgagaacaaatggacacagggaggagaatgttataccccagggcctgttggggggtggggggctaggggaacagtagcattgggagaaatacctaatgtagatgacaagttgatgtgtgtagcaaaccaccatggcatgtgtacacctatgtaacaaacctgcacgttctgcccatgtatcccagaacttaaagtataataaaacattttttttaaaaaaagggttttattgttcatattaattgatcaccattaataggatatgttgacattttgtaattcttgctgtgcactgaggttgcaccccattttttttgtttttgtttttttgctaaaaataaaaggtatgaatctaatcagtagaagacttcaaacaaatgcaacttaagagattctccaaaataacttgccagtacacttcaaaggtttcaaaatcatgaaagacaaaactaaaaaactgtcacaatttgggaaatattaaggacacaataattaaatgcagtgtgggattttggattttttttctggaacataaagaaggagattactgaaaaaatcagtgaaatacgaggggatttcaaattacttaattaatagcattgcatttatgttaatgttttggtattgatacttaccctatagttacgcttgatgttgacattacagaagaagctagtggaagagtacatgagaacaatcttattatattatgcaaattttaagtctaaaaacatttcaatgttattaaaatatataaataaaaataattaaaacataacaaaggacatggattcttatgaaacaatttcacaagattcatcatgttttcatatttgtgtttcaatcatctgttaaagacaatcctggctcccattatgtagagaatattcacttacttggtcaattctagaatatgcataaggcatattttacagatttgtagtgcattccctgaaaatgtgaaatctagtgattagagttacatatatatttttattttattttattttattttattttattttattttattttattattttattttatttattttattttactttactttgacagagtctcactctgttgcccaggctggagtgcagtggtgcgatctcggctcactgcagcctccgcctcccaggttcaggcgattttcctatctcagccccctgagtagctgggactacaggtgtgcgtcaccaagcctggctaattttttgtatttttagtagagatggggtttcaccatgttggccaggctggtctcaaactcctgacctcaggtgatctgcccacctcaacctcccaaagtgctggcattacagtcatgagccaccgtccccagccaagagttaatatttgttaagtgcacgatttctcttcaaaccgtgggtattgagttcaaattctttacttcagaattacttatgttttaacatatatctatgtcctttcagtgttgctgtcatattcattaaaattcattttagaaggcatctctctttattgtgttacagagagattgttaaatcctctcagcaaaaatatatgagaaagacaaattaagcataaagctaaaaaatatcaaatcggtttcagcgctctgaaaattggcaaagtataaaacatttaatactgtatactattcataacatgaaagaatatgttttgagtaaggaaggaaattatgtctgtagccttttgcctgggatttctcccttccatctccgctctgtcagcatgaattgcagatctggggttttaatgaggatgtcagcttgcagcttgcagtcgaagggagtggacttgagttgaggtggagagtcaagcaagatccttcagtgtttccagctaaatgtgatgaattctgcaggaaatgaacagagcaagctagttcaaactgagggctctagctggggcaagtggtacaccagctgaaagttactagtggactcctggaagtgatggaatgatagaattgctaaaataatgtctgcacagatttctggtgacttaaaagctgccgttatgaataacagggatcaaagggggtgcagtgaaaagtaaaacagagggagataagaactggctacattttgtatacacttttcagaacacacacagatgaataggtttatgagtttcacacatttgggaaaaacccattgctatgatcttcttttccaggaccttagccagccagctattcagaaatctatatgtatacttgactccagacacttctctatctacactaatttgatgaacatgtgctctgctcagatgtaagataactcaaggtagtatttgacagccatgcatgaccgttgccatagtgtggacacagtccacacttacttacacaaacatatgatgccaagccattcaagaggaagcccagcttgttctcatttttgctttgattttctttgtttttgcttattttcttttttttctttttctttttttgtattatctctctggcattagctgatcaggaaaacccatgatatcatagagagagctgatgcagaggtgttaagttgagagagaaaagtgatataaggaactggaacatctgtgatggaaatgaagcatgccttctgaatctgcttgaacccagtcactaaactaccatctgcatcccaatattgaatggtgctgagcttcacctgatcttaaaattggtgagagtgacattctcagtttatgaggggcagcttagtcacttaattatttagtcaaacagtcaactactcatggacatgcctacatggaccctgtgatattttgagagctgcattttgagtagtgagttgtttgtgtgttgtttgtttgtttattttgggggcatttcaggatcttgctcaagaactgtagagatttttttctgtgactcttttttggtgcttgcatggaggtttacagagtttcctcatctaatatagattatctagcaccaggcaatgtgctggatctcatggctgaagtgacagaggcatttgcattaaaactcaaacttactacagaatattttctttctcagagtttattcataaaagacagccttccaagttagctgataaatgggatggtatagtaaacccaagtgcaaaatgcattgtcaacactctaggatggcttaaccagtaatgtgcttcattgctagtggttggaagtacaaggtgcaattatttttccttactttggaggggataagccagcatgactcataccccttttataaacacttgacatcttctctaatgtgacaagcccttgatgttttggggcgtgcatcccaccctctagagcacatgtgttttcacaagaaattcagagttcttacaatgtccagctcatcacgtctaattaccatgatgtcatcaatatagtgttgatgctttgtggaacgttcacaaagctttttcagcctacattgtgacagagagcaggagagttaacatagtcctgggacgagactgaggatgtgagctgttattcaccccagataactgcagactctcccagagatggcgatggactctgccttcactctgcagctgtgccctggggtctggtcaagccctgccagagcctcagcggagctcgtctgcaggtgccagcagagggcgcttcacacccctcatggaaggggccgggagggcgctctcctggcaacagtgatttctgtttatttaaaccagcaggacatccccataatttgcatgtatcgttcctcctatatgtgaagaggccctgcctctcggtatcttaaaagaggttctttctctgggatgtggcatgagcaaaactgacaagtcaaggcaggaagatgtcgccatcacaactcattgggtttctgctgctctgggttccaggtgagaatatttccacaaacctaggcggagatattctttcaatctgtaatttctttcattggggactctgcaataggtgatttttggcttgattttaaaatcctaattttaaaaatgtaatgcatattctttcttcatgtctagcaagattaaaggtgattttcatacacagatatttatgttgtactgatgtttgctgtatattttcagcctccaggggtgaaattgtgctgactcagtctccagactttcagtctgtgactccaaaggagaaagtcaccatcacctgccgggccagtcagagcattggtagtagcttacactggtaccagcagaaaccagatcagtctccaaagctcctcatcaagtatgcttcccagtccatctcaggggtcccctcgaggttcagtggcagtggatctgggacagatttcaccctcaccatcaatagcctggaagctgaagatgctgcagcgtattactgtcatcagagtagtagtttacctcacactgtgttacaacccagaacaaaaactagttcagcctggctgaacggagaaactgggtgataccctagaatacttctgattgttgcaggtgctttgggggcaatgagttaaccaatacaatgaagtctggctcacccagcagagaggaaactagagtcactgctgcatactttcatctttttaaaaatgatttatttcaatagtttttgggggtataggtggtttttatttacatggataagttctttagtggtgatgtctgagattttggtggacctgttacttgagcagtgcatactgtgcccaatatgttgtcttctagccttcacctccccttctatccttcctccccagtccccaaagtccattatatcattcttacgcctttgcatcctcatagcttagctcccacttacagatgaaaacatataggttttccattcctgagttacttcatttagaataatagcctccagcttcatccatgttgctgcaaaggtcattattttgttctgttctgttttatggctgagaagtatttcgtggtgtatatacaccacattttctttatccacccgttgcttgattggcacttatggtggttccatatttttgaaatggagaaatgtgctggactaaacatgcatgtgcatgtttctttttcctatactaactttttttttctttgggtagataagaaaaataagtactggaattgctgaactgaatggtatttctacttttagttctttaaggaatctccatactgtttttcatagtggttgtattagtttacattcccaccagctgtgtaaaagtgttccctcttcaccacatccatgccaatatctattattttttgacattttaattatggccattcttgcatgagtaaggtggtatttcaaggctatggttaccaaaacagcatggttctagtataaaaataggcacatagatcaatggaacacaatagagaacacagaaataaacccaaatgcttataaccaactgatcttcaacaaagcatacaataacaaacagtggggaaaggacaccctattcaataattggtactggaaaaactggcaagccacaggtagaagaataaaactggatcttcatatctcaccttatacgaaaatcagctcaagatgaatcaaaggcttaaatctaagaactgaaaccatataaattctagaagataacattggaaaaactcctctagaccttggcttagtgaaagaattcatgactaagaccccaaaaggaaatgccacaaaaacaaaaaataaataaatggaacctaactaagctaaaaagcttctacatagcaaacagacaacccacaaagtgggagaaaatattcacaaactgtgcatctgttgaaggaataaccagaatctatgaggaactcaaacaaatcagtaagaaaaaaacaaataatcccaccaaaaagtgggcaaagaatatgaacagacaattctcaaaagaagatatacaaaccgccaacaaatacatagaaaaatgctccacatcactaattatcaggaaaatgcaaattaagaccataatgacatactttcgtctttacccatatttactttcaaactacatggacagttgttgaaggtcacctctcccttttctttccataaactatcttttacaagttggtaaaaactttagatttctcttcagagctacagtttctcatttatagcaaaagagtttaaaagggtaaagattaggaaacaagcaggtgatggcctagagctatagtgacagaagatcccatggattgaggtttcagttattgtgggttcacgggtgtgacaaattaattctatttccaaagcagccccctgaagcatgatgtttgttaagtcagattaacgttaaggttcactttcaccagtgcggcattcaactgagaattcaggaaatgctgaatatttgggttgcgatttctgaaaactggtccacggaaaatgtaactatagacatttctcttgggattttgaaaaggagacttttccaaaaagaacatttacctggaataaaaaaccagaaggatccagagccctttgttgccagtctagggagcaggacaagattccaggcccaaggaagttgaaattaagaatcctcgattccctaataagaataacttcaccaaaagttgagtgtaccaaggcactaacatgtcagagaaaatagtctgggagctcagatgaggtggaaaactcaatgggcattttatgttatatcttgccctgacatatgaaatacaggggggcaaccctccaccctgagagtaaatattcttttctgtgtatcagaggtattgtttatgtcctctttcatccacctccaaaatccaaactgcagtttgaattttctttttttaaaaaaaaaatttcaccattcttgattataggaccagtatcctgctcctagaattttttaataccaagagcaactcagcttatttgttttactttgtttcctgtgcacattaagtcactcattcaaaaataatttttggcatacaatgtagtcattgagaaaacagacatatcagatttggtgatatttttgtgagtgactttcaccgtatttggtcacaaaaagttatatcggttttcaatacattttttatcacatatattttacaccaaagtgcaatgatctactacaagaaattgtatttctacattatggtatcaggcagacagtcaccagttctttcacagggtagtttcaagttgcagaccctcatgtagagaaactcaaattgtgtgccatgattggttaaacccaaatggcaagaaaaggtgaggaagaggtaacattttgtgagatacttttgtttgaatgtctgtgagctgtttgtatgtgtttagaaacatgctgtttccaacccgtattccactcatgctatgactattcccaaagcttccccatcaggactttcctcttgcatcaaaacccatggaaaaaggaattactcatagtcatgtctggtcctgatattggatgcttgcctgaggtcactcatcacaccctcccccaccttccagggacagacaccctgaccctctccatcaagcccctcccactgtgagggcctttcttctgcctactggacatcttacatgaaaatcgagtttatctaatttcaagatgatgcttgttactcctatatatgtgtttctttcatgtccagtggatctttttcaactataaaagtagttaattgtctttagctgaggggaagccatgatatcttcttcaataaaaaataaacatatttttgcatttaatggattttaacataatatcggagttttcaggaacaattcaaagccatcatgtgagggttaggagcatttgagtaaataagacaatttttgatcccaagtactgatattcagtagggaaatgagccattcagagaacaatacctacacagtgaaagtgaaaagaatcatttcaatagctgataaattgtataaaattcaggcagtggcatgtggtatctggaggccgagaccatttatttatgcggaccagggaaggtctcggggtcatactggagatgcttctgaacggtgaggaggcagccaagtgaccataggaacagcaaagaccataggatcatcacgagaagggcagggactgggagatttcaggtaaaccattgtgcattgaaaaagccaaccagtaccataataataagatgtcttctgtgattttattcctttaaggagaaaatttatactaatatctttcatcaaacaccttgacctgggtcacacccataacatgaaatgttccctggctcagaagctggaagttcagttttgcatccctgttgtaagtctgcaggctccacaaagcccctccctgccactcaagcccttatcagtgggttggttgctgcctttagggtgggatcacctgaggcagaggaagcactggacctggggctctggcccttgggtcctggcatcagctatgggagctccatgtgacagggttcttatgtcccgtgctgagatacagaccatcgctcagcaagcccagcattcatctcccgcttgatcagccaacacgagtctctgggaggcctgtagagtgagacatcattaacactggggaagagttgtgttttgtttccacctcagattccagtggcaacattgtgggccccagattccagcttctccctcagtatctccaagacagagagagagtttccatcaccagcctagaagcagatgaatccagggaaggtttcaaagatccacccatgtgctttgtctacattggccatggtccacccctgcttggcacggtggtcctggggcagacacttccttaactttcagcagctcgagtaccctgatgacattgctgattattattgtctgaaactgtatcctctcacctggtaaacacttgcagtgcccagccacaaataatgtgaattagaattaaaaattaaaaacatgttttctcagttacactagctacatttcaagtgttcagtagccacatatgactaatggctaccctattgtacagcataaatgtagacatttttattgtcttagaaaattattttgcttaaaaccgctctaaatgttgacaagtgttccctcattgtgttatagctcagagcataaatctcaccagccgttagtctggaaaactgggagtcctcagaagctctccagctggtgcaaccactgtggtcctcagatctgctctggaagagtttccagaataacgggaatgagcctgggctgacagatccataaaagaggaccttggatttcctctccagcccctgccattatgcccggcagggtctctcacacccctttttctctcttccaaaactacattttcagcatttcacatggatttcagaacctaattcctaatcgttttgtgagcaacatcttttctggatatcccttgtcctcaactttgggactggtttatcaaggagaggtgtcattctgtgttccttataggatctggcctactgatggatgtaataggatctgcttcatcattacccatgaaaagactcaccgtcaagattgactgggactcagcatctaaaatcctataagatgctatgtcaccaaccagccattagatggcagacaaaccccacagtaaacaccagaaataagcctgatcttagaaatactaggaaaatcaacagggatattttagggctaaaatgaggtctcatttatgacctagattacatgggaggagctgccagtgcactgagttgtgggaaactccctctgtgctctgtgctctgagactggaagcccagccttttcctccccaccgcgttggctgtatccccaaaccctacctgatgtgggctgaatccaggcagaggggaggctgccaatggtccctggaatggtttctccctgttaccacacagccactgggccatgtgtgctactctgtctcacaaaggccaccaggggaggacctgcccaccctgagctctggggacaaaagtccctccagttggggtctagaaccactgcccatctccccagcacctgctgctctgtgattccccagacccccgtcaggacagtcagtgtccttagcaatgggcagggaggtaccgctcagcccagaatggatgtaggtttggtcctgagcttcctgaccctcaggctgtgtagtgatgaaggggccatggggtggtgcaaccattgctggttttaaatgtttgtgctcaatttatcaaagtttaaaaatcatatcttacactgacaattaaagttatatctattaacatataagtgtgcatattatacttattcctaatatagatgcacagtatatccaaatgtataaatataatttatatctaaaatattatatgtatatttaatatgtaagggttacattacaaatatatacctatgcatgtaattttatgtttgttaattacttatatctaaaatattatatgtatatttaatttgtaagggttacattgcaaatatatacctatgcacgtaattttaagtttgtttatttagcatgtgttctttttctttctaaccagaacagagcctggctgagtaaagactctggggacatttgctgttcctccttctttgactccagcagggccccagccatgcagaatcagtgaggacagagctgagagcagccagctccaggagctcaggcccagccctaagggtcgtgtatctgagactttcacactggcagtggactctatgcttggtgcagcgcccatagaagtatgagcagtttccttccctgaaaccctgccaggcagctctgtgggcaggacctttggttcctcccaagtcctcagccccatggctcaagagagcagctacttcctccacagcccagggccagagcccagcagtctcaagttgtgcaagcttcaccttagtcctgggttgaggaccctattccaaatctctcctcatttattcccataactgaaagcctgtcctggtcttaaatgcacaggccacatttacgcaattcttaaagctaaagatgtcgtatgagaaatcagaaatttgatttcattttcatcctcagagcctggcttcttccagctgtatcagatcgaagtgttcatacgttctcctccctatacaacttaacttagaagcacagcgaaatttaaaatgtgacaaagctcttggcagctatgcagcagtcatccccttcttcctttggtgtatagggcaccaactatgtcttgccgtacatggtgagggtggtgagtttctcccagctcaggatgggagcagggattaagggcacatgtgatcagctccaaaatgataatgtcagaggagtgggcagggatcatgggaaaatggttatacctcagaaaaggacagaaagtgaagagctttgctttgcatttcttcctgtaacagttaagagaggatatgatgcttagagctgccgcaatcctcttgagaccatggggcatttacaacaagaatgaaaagccagtgataatgcaggtgcaaagcaaaaatgtagtaacaatctggggcctttcagctgtcaccaagctgttgtaccaaccttaagtgcttcaaccttcagacttcttgtcattacttaaaccattactattatttctttgacttgtttctaaaattattccaacttatctataaaagacacttaagagaaagatcctggctgggccacagactgtgcttcagaagaagaaacatattatcagaagtgtgtgtgtttgtaagagtctgaggcatgaagggcaggaaacatgataagtgatattctccctggcaccttcgtcctgctatgcccatggcaagagaaacccaaacaatgccaaagagttcctcaattctgctctttcattatctccatttctccttttatatcctaagcatgaaacatccctttgttctccttaattcctcccttttccaaggtcatgaattgttgtcaagaaagagacaggaaccgtttgaaaagataaaacctggtgatactgtgcatttcctcaacaccaacatggttctgcaagtttcctcccttctcagtggttttcttatgggaagttgctggctgcctcagccaggtctctgtcagaggttgcatttggagcgtttactaagcaaagcttccaggtagttagtgctggattcccaggagagtagcaggatggtgggtctgtattcccagcatgcaggaggccagaatgagacctgggggaaggctgtgggtgtgggaagaatggatttagaactcagacctgtagccacggcctttggaacccaatagtgtacactaaacagatggagctcaggggaaatctggtttaaaggtgttatagtcatttgtcatcttgtttatgtttctagtgctacacaggaatggatttatggaagtttttattgtggaaataatgtacatgaaaccccattgcctatagtgagtcacatgttagttgtagaataactattaaagaatttgatttgaaaatgacatatggttaataatatcttccatagcctctttttctaagatactcaagggtgcatttaaagaaaactgggtatataaaatgtgcatataatgtgtgtgtgtgtatgtttatgggcacacatatacactcttcagggtgcatcatttggttaaactctcacaataccccatgacttccaaagtgctccatttcacatatgagagaaccagctatgagagctcatgactggtttgccaaaagtcacatggtcagcaaatgcccaaagtcacatggtcagacttgggattgaagcccaggtctgtctggctttagtatgttccttctacgtggccactttcatcccatggttgagcccaaagcctataaataggaagaagggaccataaaaacagtgtggaatccacagctccctgctgcctctgtctcatgccaggctggccctaatcttaaactagccccttctgtggttttctcttcaaaatataaccctctcg

SEQ ID NO: 81 - DNA, 885 bp, Homo sapiens
ctgcagctgcgcccagcctgccccatcccctgctcatttgcatgttcccagagcacagtctcctgacctgaagacttattaacaggctgatcacaccctgtgcaggagtcagacccagtcaggacacagcatggacatgagggtccccgctcagctcctggggctcctgctgctctggttcccaggtaagaaaggagaacactaggattatactcggtcagtgtgctgagtactgctttactattcagggaacttctcttacagcatgattaattgtgtggacatttgtttttatgtttccaatctcaggttccagatgcgacatccagatgacccagtctccatcttctgtgtctgcatctgtaggagacagagtcaccatcacttgtcgggcgagtcagggtattagcagctggttagcctggtatcagcagaaaccagggaaagcccctaagctcctgatctatgctgcatccagtttgcaaagtggggtcccatcaaggttcagcggcagtggatctgggacagatttcactctcactatcagcagcctgcagcctgaagattttgcaacttactattgtcaacaggctaacagtttccctcccacagtgttaccaacccgaacataaacccccagggaagcagatgtgtgaagctgggctgccccagctgctcctcctgatgcctccattggctgagagtgttgctcagatgcagccacactctgatggtgttggtagaggggtacgtgaaatcgcctctgcaccctaattcttttctctttctcagccccaactgcacagacatagcaatgcatctcctgatttgataaatacagagatcatgacacttgaggagtctagtttatggcttcagcttgaa

SEQ ID NO: 82 - DNA, 2167 bp, Homo sapiens
gcatttgtgcctgaagctgccgggtctgctacggcaccgcggggctgcagaaacccgggggccaagggcgggctgcttgccgctatggctggcagtcaggacatattcgatgccatcgtgatggcggatgagaggtttcatggggaagggtatcgggaaggctatgaagaaggcagtagtttgggtgtgatggagggaaggcagcatggcacgctgcatggagccaaaatcgggtctgagatcgggtgctaccaaggttttgcttttgcatggaaatgtctactgcacagttgcaccactgagaaggacagcagaaagatgaaggtcttagaatcattgattggaatgatccagaaattcccttatgatgaccctacttacgataaactccatgaagacttagacaagatcagaggaaaatttaaacagttttgttcgttactcaatgttcagccagactttaaaattagtgcagaaggttccggactttcattttgaggaggatggatgaacagagaccgaacgtcgaggaacagatgtgtgtgtgacgtgtttagaaatgcggtgaagggccagacggtgctgggaaggcagttgttcattgggagggtgagggttccggttcggccgtgggagggcttccttccctggggttttctgcctgtgtcaccttggtgcccgtcttggggcctctccacacatgccctttgttgggctgaagccgtccctggcagagccctcgtgcattgacttgacagcctctccggcagcacaggcctagctggttctgggttggagttggctctggatagggttagtcaccaggcctggactgaaggcagttatttttattattattattatttgcaatgagagagatggttggccccgaatgaggctcatgggaggtttggacgggtgctgtgccgcatgtcgaggccgattgtgtgccaggcggtgcgggacgtgcctcccgtgtgttatttaatcccttcaggagcccacaagatgggtgttattctcattttacagaggagggaggggagacgcgaagggattgcctggtctaagggcacccagcagcagagctaggacttccgccctaaggctgtgcctcactgccaccaggcacagccgcctccggaatgcacaggcgagtccctgccctccctcccaggccgcacaggtcctgccaagcctcacggagcacgggggagtctgtggtggccagtttacctgggcatctggagacgttcttcgccgagagtcgtcggggtttcctgcttcaacagtgcttggacggaacccggcgctcgttccccaccccggccggccgcccatagccagccctccgtcacctcttcaccgcaccctcggactgccccaaggcccccgccgccgctccagcgccgcgcagccaccgccgccgccgccgcctctccttagtcgccgccatgacgaccgcgtccacctcgcaggtgcgccagaactaccaccaggactcagaggccgccatcaaccgccagatcaacctggagctctacgcctcctacgtttacctgtccatgtcttactactttgaccgcgatgatgtggctttgaagaactttgccaaatactttcttcaccaatctcatgaggagagggaacatgctgagaaactgatgaagctgcagaaccaacgaggtggccgaatcttccttcaggatatcaagaaaccagactgtgatgactgggagagcgggctgaatgcaatggagtgtgcattacatttggaaaaaaatgtgaatcagtcactactggaactgcacaaactggccactgacaaaaatgacccccatttgtgtgacttcattgagacacattacctgaatgagcaggtgaaagccatcaaagaattgggtgaccacgtgaccaacttgcgcaagatgggagcgcccgaatctggcttggcggaatatctctttgacaagcacaccctgggagacagtgataatgaaagctaagcctcgggctaatttccccatagccgtggggtgacttccctggtcaccaaggcagtgcatgcatgttggggtttcctttaccttttctataagttgtaccaaaacatccacttaagttctttgatttgtaccattccttcaaataaagaaatttggtaccc

SEQ ID NO: 83 - DNA, 1914 bp, Homo sapiens
ggcacgaggcgtcctgttgctggtctccgtccggtcgccggccgtctaggtctccggccctccccagccgctcctgcgcccttgccggccccgccgcccgcagccctggcgctccctgcgggccccgccgaggccgcctgcgccctgtgccagcgcgcgccccgggaaccggtgcgcgccgactgcggccaccgcttctgtcgggcgtgcgtggtgcgcttctgggccgaggaggacgggcccttcccgtgccccgagtgcgccgacgactgctggcagcgcgccgtggagcccggcaggcccccgctcagccgccgccttctggcgctcgaggaggcggccgcggcgcccgcgcgcgacggcccggccagcgaggccgcgctgcagctgctgtgccgcgccgacgccggcccgctctgcgccgcctgccgtatggctgcgggccccgagccgcccgagtgggaaccgcgctggaggaaggcgctgcgcggcaaggagaacaaggggtctgtggaaatcatgagaaaggacttgaatgacgcccgggacctgcatggccaggcagagtcagcagctgcagtgtggaagggacacgtgatggaccgtaggaagaaggcactgaccgactacaagaagctgcgggccttctttgtggaggaggaggagcatttcctgcaggaggctgagaaggaggaggggctccctgaggacgagctggctgaccccactgagcggttcaggtcactgctgcaggcggtctcggagctggagaagaagcatcgcaacctgggcctcagcatgctgctgcagtgatggcgccaacccgtggcagtcccagagctggaggcaggaggatggatcctcatctccatgggaagtgtcagcgtgtggctgccagggaagcgtggcaggcgcctggccttgggtccatctacatagttgcgtgtttcaacaatgtccatttatccttcaccctgaggcgtgttttgggggctgcaaacacctcccggtagaggctggacctgaggacccttcccacctgtgcccgtcccttcctgaagtcctagccacagcccatcctccatgagtcccggcagctctgggtcatgcccttccctggtcacccatctgcccctcacctcgtcatccagggacccagaccctgcaccttccatgtgggcccacagatccttggcaggtacctgaggtgcaccattgagtgtcggatttggggttagcatccagaaagaagaatgcgcatgacgctctgtgaaggctggaactcaggtcttcagggagagaaaggaagactggattgcaccttgatgcctcctgaggaggcggcccccctcttgaggtgggcgtgggcccggcccagccttatccaagtcgctctgtccacctcccccttcctggcccccaccccactcctgtgcctcccaggagccctccctgtgctccacctgcctccgcagaaggaagcctctttctctgtttccctgggtgagggggctggcaggtggctaaccccatttagcatctccaggccctgccatggtgtctcatcttgctgttatctctagctctttccctcctcccatttcctttagtagttgaattttgcaaagcttgtagcagtagctcagttgcctgcagcatccttgtgtgtagataaattagtcgacagaaactcagcactggggacaggattgcaaagtcggggacatagatgcagacagttgttgagatttggggatagccgggcttgtgagcggtgcccatttccagatgaagcctttcagcccttctgagtccccggcccttggtgcgatgtctgtgagtttgacctgcccagcgtgtgggctggctcaatgctgaataaagtgggtttgtgtcaaaaaaaaaaaaaaaaaa

SEQ ID NO: 84 - DNA, 1119 bp, Homo sapiens
cggccggccgcccatagccagccctccgtcacctcttcaccgcaccctcggactgccccaaggcccccgccgccgctccagcgccgcgcagccaccgccgccgccgccgccctctccttagtcgccgccatgacgaccgcgtccacctcgcaggtgcgccagaactaccaccaggactcagaggccgccatcaaccgccagatcaacctggagctctacgcctcctacgtttacctgtccatgtcttactactttgaccgcgatgatgtggctttgaagaactttgccaaatactttcttcaccaatctcatgaggagagggaacatgctgagaaactgatgaagctgcagaaccaacgaggtggccgaatcttccttcaggatatcaagaaaccagactgtgatgactgggagagcgggctgaatgcaatggagtgtgcattacatttggaaaaaaatgtgaatcagtcactactggaactgcacaaactggccactgacaaaaatgacccccatttgtgtgacttcattgagacacattacctgaatgagcaggtgaaagccatcaaagaattgggtgaccacgtgaccaacttgcgcaagatgggagcgcccgaatctggcttggcggaatatctctttgacaagcacaccctgggagacagtgataatgaaagctaagcctcgggctaatttccccatagccgtggggtgacttccctggtcaccaaggcagtgcatgcatgttggggtttcctttaccttttctataagttgtaccaaaacatccacttaagttctttgatttgtaccattcttcaaataaagaaatttggtacccaggtgttgtctttgaggtcttggatgaatcagaaatctatccaggctatcttccagattccttaagtgccgttgttcagttctaatcacactaatcaaaaagaaacgagtatttgtatttattaaactcattagtttgggcagtatactaaggtgtggctgtcttggattcagatagaactaagggttcccgactctgaatccagagtctgagttaaatgtttccaatggttcagtctagctttcacagtttttatgaataaaaggcattaaaggctg

SEQ ID NO: 85 - DNA, 520 bp, Homo sapiens
caggctcgaggcgtctgccgcacctcagcccacgacctgccccgctgggaggtgcgggccgctggccaggccctgaccgcaacctggcccagaggccccagccctcaggcaaggttctccggtgaagccacagcctggccacctgtcttgatctccccaccgagaaggccccgcccctcccgctgcagccccacagcatgcagccccaggagagccacgtccactatagtaggtgggaggacggcagcagggacggagtcagcctaggggctgtgtccagcacagaagaggcctcacgctgccgcaggatctcccagaggctgtgcacgggcaagctgggcatcgccatgaaggtgctgggcggcgtggccctcttctggatcatcttcatcctgggctacctcacaggctactatgtgcacaagtgcaaataaatgctgccccgcatgcacgcggggggctggccgcaaaaaaaaaaaaaaaaaaaaaaaaaaaaaaaaaaaaaaaaaaaaaaaaaaaa

SEQ ID NO: 86 - DNA, 894 bp, Homo sapiens
ggcggcgctatgctgtcctgcttcaggctcctctccaggcacatcagcccttcgctggcgtctctgcgcccggtgcgctgctgcttcgcgctcccgctgcgttgggccccggggcgccccttggaccccaggcagatcgccccccgccgccccctggccgcagccgcctcctcccgggaccctaccgggcccgccgccggcccctctcgggtgcgccagaacttccaccccgactccgaggctgccatcaaccgccagatcaacctcgagctctatgcgtcctacgtgtacttgtccatggcctattacttctcccgggatgacgtggccttgaacaacttctccaggtatttccttcaccagtcccgggaggagaccgagcacgcggagaagctgatgaggctgcagaaccagcgaggaggccggatccgcctgcaggacatcaagaagccggaacaggacgactgggaaagcgggctgcatgccatggagtgtgctctactcttggaaaagaacgtgaaccagtcgttgctggaattgcacgctctagcctcagataaaggtgacccccatttgtgcgatttcctggaaacctactacctgaatgagcaggtgaagtctatcaaagaactaggtgaccacgtgcacaacttagtgaagatgggggccccggatgctggcctggcggagtacctttttgacacacatacccttggaaatgaaaacaagcagaactaagccacgagctgccttcctcccaggctagtggatccaaagaccaaagtcagctgtctcctgctttcttgcccttaaaatcacctccatctttatattcttctgttatactattcctccaataaagtgatttgtagaaaaaaaaaaaaaaaaaaaaaa

SEQ ID NO: 87 - DNA, 1613 bp, Homo sapiens
ggaagaggaggcttgaggcccagggtgggcaccagccagccatggccacagccgagaccgccttgccctccatcagcacactgaccgccctgggccccttcccggacacacaggatgacttcctcaagtggtggcgctccgaagaggcgcaggacatgggcccgggtcctcctgaccccacggagccgcccctccacgtgaagtctgaggaccagcccggggaggaagaggacgatgagaggggcgcggacgccacctgggacctggatctcctcctcaccaacttctcgggcccggagcccggtggcgcgccccagacctgcgctctggcgcccagcgaggcctccggggcgcaatatccgccgccgcccgagactctgggcgcatatgctggcggcccggggctggtggctgggcttttgggttcggaggatcactcgggttgggtgcgccctgccctgcgagcccgggctcccgacgccttcgtgggcccagccctggctccagccccggcccccgagcccaaggggctggcgctgcaaccggtgtacccgggggccggcgccggctcctcgggtggctacttcccggggaccgggctttcagtgcctgcggagtcgggcgccccctacgggctactgtccgggtaccccgcgatgtacccggcgcctcagtaccaagggcacttccagctcttccgcgggctccagggacccgcgcccggtcccgccacgtccccctccttcctgagttgtttgggacccgggacggtgggcactggactcggggggactgcagaggatccaggtgtgatagccgagaccgcgccatccaagcgaggccgacgttcgtgggcgcgcaagaggcaggcagcgcacacgtgcgcgcacccgggttgcggcaagagctacaccaagagctcccacctgaaggcgcatctgcgcacgcacacaggggagaagccatacgcctgcacgtgggaaggctgcggctggagattcgcgcgctcggacgagctgacccgccactaccggaaacacacggggcagcgccccttccgctgccagctctgcccacgtgctttttcgcgctctgaccacctggccttgcacatgaagcgccacctttgagccctgccctggcacttggactctcctagtgactggggatgggacaagaagcctgtttggtggtctcttcacacggacgcgcgtgacacaatgctgggtggttttcccacgaatggaccctctcctggactcgcgttcccaaagatccacccaaatatcaaacacggacccatagacagccctgggggagcctcttacggaaaatccgacaagccttcagccacaggggagccacacagagatgtccaaactgtcgtgcaaacccagtgagacagaccgccaaataaacggactcagtggacactcagaccagctcccagatggccctggacagcaggagagggtgtgggatgaggcttcccagagaccctgggtctagaaagcggctcctgaaggtcccttattgtggctgatattaactgtcaatggttatgggtcctataaaaatgcccctcccagataaaaaaaaaaaaaaaaaaaaaa

SEQ ID NO: 88 - DNA, 14709 bp, Homo sapiens
agggaattctctggggctttggggaatttagtgcgtgggtgagccaagaaaatactaattaataatagtaagttgttagtgttggttaagttgttgcttggaagtgagaagttgcttagaaactttccaaagtgcttagaactttaagtgcaaacagacaaactaacaaacaaaaattgttttgctttgctacaaggtggggaagactgaagaagtgttaactgaaaacaggtgacacagagtcaccagttttccgagaaccaaagggaggggtgtgtgatgccatctcacaggcaggggaaatgtctttaccagcttcctcctggtggccaagacagcctgtttcagagggttgttttgtttggggtgtgggtgttatcaagtgaattagtcacttgaaagatgggcgtcagacttgcatacgcagcagatcagcatccttcgctgccccttagcaacttaggtggttgatttgaaactgtgaaggtgtgattttttcaggagctggaagtcttagaaaagccttgtaaatgcctatattgtgggcttttaacgtatttaagggaccacttaagacgagattagatgggctcttctggatttgttcctcatttgtcacaggtgtcttgtgattgaaaatcatgagcgaagtgaaattgcattgaatttcaagggaatttagtatgtaaatcgtgccttagaaacacatctgttgtcttttctgtgtttggtcgatattaataatggcaaaatttttgcctatctagtatcttcaaattgtagtctttgtaacaaccaaataaccttttgtggtcactgtaaaattaatatttggtagacagaatccatgtacctttgctaaggttagaatgaataatttattgtatttttaatttgaatgtttgtgctttttaaatgagccaagactagaggggaaactatcacctaaaatcagtttggaaaacaagacctaaaaagggaaggggatggggattgtggggagagagtgggcgaggtgcctttactacatgtgtgatctgaaaaccctgcttggttctgagctgcgtctattgaattggtaaagtaataccaatggctttttatcatttccttcttccctttaagtttcacttgaaattttaaaaatcatggttatttttatcgttgggatctttctgtcttctgggttccattttttaaatgtttaaaaatatgttgacatggtagttcagttcttaaccaatgacttggggatgatgcaaacaattactgtcgttgggatttagagtgtattagtcacgcatgtatggggaagtagtctcgggtatgctgttgtgaaattgaaactgtaaaagtagatggttgaaagtactggtatgttgctctgtatggtaagaactaattctgttacgtcatgtacataattactaatcacttttcttcccctttacagcacaaataaagtttgagttctaaactcattagaattgttgtattgctatgttacatttctcgacccctatcacattgccttcataacgactttggatgtatcttcatattgtagatttaggtctagatttgctagctccaagtaattaaggccatgtaggagagcatggtaaccacagatagaactggtattatcccaagtggtctgcagactgctgagtggggatgggatctgctctctgttgagagttggtaatcattggtttgaaatgtgatgaaaccactcaagccaatgaaggtgggtgtgtaggtggggagtactttgccataatattttaaaacattacctggttagagttctaagtggtacttatttttgtttggttaggggaaagcctgaataaaaacagaaatggacacataatatgcatattccatagtctttgggaggctggaatgtgcctgggatttgggtctaagtgtatgcgtaattcttacctcactaaagaatttgccttgtttttttccttttggtgagtgactaaaacgtctgggcttccctgtgtgcgtgctacagtaagcaagcagaggctgtgcaaaggtgtgagcaggatcacgtggaatctggaggatacatcttggcttgcaaactgcctctgtctcctgggtgggactgttctgtccttgcactgctgttctgtgttacctcttggggtgtaaggttttgcttacaggagacaaactttgggcgtagaatggaagccactgccagcctctgtgctgagaaggaaggtgcttgtttcaaagggagcagcaagggaggcttgttctactcacctgggcctgtttgcctgagaaggggagataagggctgaactgggactagccagggggaccaacacaaatggtgggggatcatgacctgaaggattctttccttcccatgagctgcagggctggttgccgtccttgcaactgtgtcttatttgcctgtgccgttatatcttggtgacccctccacgtgtacactactgacaaacgggtggagtgctggggagaagtcactgtgccgcccacctagtaaaccttctgtctgtgctcatggcatctccaagatggggcactgctgtgtgcagaatccagggtcctctttctgcttgcaactcctttccctggatgccccagaaacaatccaggcctcctttcctatcttacccctttgctttgctttttaccccagcacctctataaccgccttctcttcttttcagaactccttgtttctcatcctgttttttatgattacaaaactcttgcttccaccctggaagataactgctatagatgcctgtatgtaaatggtgctgtctccagcaactggcatgctgaagaagaattgattcacggggtataaatgttggggattggaagtggggatgaaatggcacttgttgatacaggagcagagaggtgaggccgactgctgaagacagctcgccaccctccttgcctccactccaatccaggggctggggccacattctttgccttcatttatcctcagatcaggtgagatcgacaggaggtgttgatggcagtgccagcaattattgctaatccgtttgcatccttatgcatagatctgaattcagactttgtgaatttccagaggtgtgggtaatataatagaattcagtgagtgggcatggctgatcttgtgcaaattaaaagttatggggcataagaatagcaaaagttgaacttcttttaaaaaggaaagtaccctgagagccagtattggttgaggctcttcagtatgcccaggttggcagcactgagaaccgcaggaacggcctgttgttacaaaaaggagattgactcagctgcccttggtgcatctgactgactatgactgctgagagattccaaggacccttaatgccagggctaacctctccatgtgcagtgagacctctggaggaagtgtcatcctctggctttgtgtggtactcattatggtgcagtgcgggcatgaaatgaagacacccaaataggcttacagatacgatatgttttaaatgttcgtatttaacaaaaacatactgacactgtttggaaatggcaacaggaagatagcaaaatgaatactaacattacgaaaagatgaacaggtacatgttccaaggcaggtggctgtgaacttcctctgagtgaaggcatcccctccagcacctttcagcctgctagttaggacgacccgccgccaccctccaggacctccagccctgcactgcctttcctctcttttaaataattcttcattgagttctaatatgtaaaaaaaaaaagtttactgtaaagtttgcaaataaggaaattttttttaaaagtcctcagtaatcttaccagtaacaattgttatgggcacatttgcttttggaagatttcttttgtatgcatgggataagtacatttttaaacaaaaatgggattatgccataaattctattttgtgactttaatatatagtgaacaccttttttaatgatgacaggatgttcccttgcatggctgtatcaatttaaacaatcttgtttcaatgggcatacagggtattttctagtttttttttcctcttagaaaataatacttgcgatgactttccttgtagctcagactttttcacgtctgttgttatctctttgggaatgctgaatacatacatttcgagaaggaaatgactgttaaactcttaagacttcaggttcatattgctaaactgcccagcagggagggattttttcaattagtgttctcactggtgaggcaaacctgatgccttcccctcttcctcagaaccggctttatcacattgaaaacctttgctcctccgacggatcgagtctgctttccctgtggatgtgagcattgctttgtctgctggtgactgaacatctctaccatgtgtcaattggccatttgtggtgtgtgtgtgtgtgcgtgtgtgtgtgtgtgtgtgtgtgtatgattttctaattcctagtcatttttctattgattgttttgcaaaagccatttacatcttaaggatattgataatcttttgttatatttgatgcaaatatttttttccagtttataggttgccttttaattttgtgtttcaggtagataaaagttaaacgattttcttaggttagtttatcactgtggtttctgaacttgttatgtgtagatcttttccaccccaagagtacataaatattaatccatactttcttatggaacttgtatggtttcgttttttacatttaaaccttcttccccgtggtgtgtgttgtggaatctgtgtttgtgtgaggaggggcatggtgctctcagaacccacctcctgtggccagagagccctgtcctgtgagggtggttatcacagtggcagggttcaattcagaagaccttgagggcaggctgatgtttcctgaatgggcccctggttgttgcttgtccctgactctccatttccccatctgagtggatttggacctaatagggcactggagctggttcgaatcctgactggactacttggcaactttatgtctgggagcaagttacttaacctccccaagcctgtgtctgtgaaatgcgggtaaatgaatgtagatgtttggcagcagctactccttgttgagctctcacagtgaactctcctgcctctgccctccttccccgcctcccctggtgcctagcgtcaggtctagccacttcctcctgggcccctctcccttttctgtggctggctgcctgcccgcctggcgctggacctttcatgtaacgggaatcagcatgtatattctggtctggtctgtttctacacttaattttgtttccagtagtatttccctgtaccggcagagttcacaaacacatttgaagaggctttttctcaggattcttaaccttcccaaaggaagtcccatggatgggtttctagaagtctataaatgctctgaaattgtatttttctgtggaaagcataactttcatctgcttgttcgtgctcaaaaaagatcatgaatgaatgattgcatgattttatgccattgtgcttatactaaaggatatgtagcccatctcttgagctgttaaactgttttgactactttaaatcgtgcagctgtgagcatctctgtaaatttagtgtacacatgtatcccctggagtggcattgcctcggcagtgagcacttatggttttataactctcttcacagactcaaatgactccagaaagctacacttcctgttgtgagtatatgatatccatttccctacatagccactaacatcaggtttttacaattttatttatttcttgctactttaagaaatttttgtggtgaaatacatataatagaagttgactatctgaatcatttttaagtatacattcagtagtgttaagtatgtcgccattgttgtacaaccaatctccagaactttttcatcttgcaaaacaaactctgtacccattaaataacattaaacattccattccctccagcctcagcaaccccattctactttctgtttctgtgagtttgactattccaagcacttcatatcagttaaatcatgaagtatttgtctgtctgtgactggcttatttctctgagcacagtgtcctcgagatgcgtctatgttgtagcatatgtcagaatttccttcctttttaaaagatccaaataatattcttattttatatcttttttttatccattcatccattagtggacacttgggttgcttttggctattgtaaataatggtgctatgtacaaatatctatattattgtatttacaagtataatgctgtaatgtacacacatctttttgagatcctaccttcagttcttttgagtatatagccagaagtggtattactaaatcttacgatatttctatttttaatttattgaggaaccactgtagtttttcatagcaactgcaccattttacgttctcaccaagagtgcacaagggttccgaggttcccacatcctccccaacacttgttattttctgctttttttagattgcagccatcatagtgggtgtgaggtgacatttcattgtggttttgatttgcatttccctaatgaggagtgatgctgagcatcttttcatatgcttactggtcatttgtatgttgtctttggaaaaatgtctattcaagtcctttgactattttaaaaattgggttattagagttatcgttgttgttgacttgtaggagtttctttctatattctggatattaatcccctatcagatatatgatttgcaaatatcttctcttattccataaggttactttttcactttgttgattgtgttctttgatgtatagaagtttttagttttgaaatagtctaatttatctgtttttacttttgtggtctgtgcttttggtgtcatatccaagaaatccttgccaaatccaacgttataaggtacttttaaggtattttagttgtcttagtctatatttctgtactcacctttctttatccactcatcagttgatgggcatgtaggttggttccatatctttgcaattctgaattgtgctgtgatcaggtgtctttttagtataatgatttactctcctttgcgtagatacccagtagtgggattgctggatcgaatggtttttataattttctattttaccacagtttctctctgcatttttcctctttgaccactaaccatgtgaaattctcatattgacctttataatgatcatgaactcttagtatcattgggaaggccacatttgccacttatgattgtaaaccttatcctccatttttcctgttattgttggtgcaaaaagcacctattataccaggactttaaaaatcagtctgataagtctttgataagtctaataataataactgataagtccattgaatttgcttctgattactttttctttagtagctaaacatgtatgtactcctatgattacaatgaacactcctctccatttaaattaattatttacattgatgaaatagcaaaatgttaatgactaaatactgtcttggttttttcgttccaggtcagtcaatattaacttcttataattttcttttttttctttatgtgtgtgtgtgtgtgtatttttttttttttaatttcaatggcttttggggtacaaatggcttttggtcatatagatgaattctacagtagtgaagtctgagattttactgcaccggtcacctgagtagtgtacattgtacccaatatgtggttttttataccttgcccccctcttaccctccccactttgagtctctagtgtccattatgtcactctgtatacctttttgtacccataagttagctctcacttataagtgagaacacacagtatttggttttccattcctgagttgcttcacttagaataatatcctccagctccatccaaaattgctgcaaaaaaaaaaaaaaccacaaacattattttgttcttttttattgctaagtcatattccatggtgtagagataccacattttatttatccactcactggttgatgggttggttccacatctttgcaattgtgacttgtactgccatcaagtgtctttctggtataatgacttcttttcctttgggtagatacccaggagtgggattgctagatcaaatggttcttaacattttctctctggatctatttctggaaattttaggctccagtttttgttgttgttgttaataaaatgcaatggaatgtaatgatcatcacttttcattatgctttaaaatctggtaaatggaggctagaacactcctgtaaggcaagaatattctctctgttggaactcaaatacacagaactgggtaaatctcaatcttaatctttgattcaggacacaacatggctctcttttacttgctttctttaattgttttttaataatgtggtaagcatttctgaatctcctatccaatacaaaaactaggacaatacagacagtaactcctatggttacaatgaacactcctctccacttaaattaattatttacactgatgaaattgaaatagcaaaattttaatgactaaatactgtctttgattttttgttccaggtctgtcaatattaacttcttataattttctttttttttctttatgtgtgtgtgtgtgtgtatatatatatatatttaatttcaatggcttttggggtacaaatggcttttggtcatatatatgagttctacagtagtgaagtctgagattttactacaccttccacttatgtggtcccacaccacccgcctcccctgccgcctcctgccaccccctaggccaaggtaataatcatcctgaatcctgggtttatctctcacttgctttcttttcatataattttgcaaaagaatctgatctaaatgtgtttttcagagtatatatttatattttagctgttcttagagaaaatttattattttgcatgtaatcttatggaacattctcatttaataccatggtaagattcagcccttgcccaggggatagttcatttagtttgtttactggatagagctcatcatgtgactatacctcagttagtttatcagttctcccatccatggtgactaggttgcctctcagcctctcaacaacactgtttctcagtgtccttgtagaagtgatatgtgggtgttttctccttacacagagttgaaaggtgacgacaacaacgttggcactaccaatcccccaccctccagaggggtaaccagtgttaccagtttgctgtgtttcctgctacacctcgccttattcacttccatttgtatctgaaaaacgtgttgcatggtttcttttctatagaagtggtaaaatgctattgtgtcctgtacattattgattactttttttcatttaacagtagggagatgcctgggagtacacagagaactgccctcattgttttcaacttctgcactgtatgtctgtgagtttagccattctgctgttaatggaaatttacagtattctaatcttttgatattacaaacagttctgtgcgatcatcgtcatacacaaccccttgtgcacaatgcatgagtgtttctcagggtaggtaccaagaagtgaaattcctgggtcatagggcgtgagtccgacatttttctccattctgccctgttgccctccagagtgggtgtccagctttgcatacctaagtatgagagtatctgttgttcatatcctctacgacgctccatatatgaaacttaagtttctgctagttgccatctttgatctatcatgtatgcagtgacctactaagactgtaattggtacagtagattcttgtcatctgtgtgtgaatttagcattcatgggcttaatgctgacaaggcccccagggtccaagacatataatcatgtataattttgtcaaggtataattttttaaattgcttttgtcatgtgtctgctggtgatgcccaacccagtgctctgcacccaggtcacactgtggctttgtcctctgcttatgcctgcattgcagcaactgtcctgaagagaccaaaattatgcagatttaggtaagtccatggctaatgttattatattatgtgctattgtaatggatggggctgtggagtgtatgaatttataaatcactggtcttgtaattaaaattcaaacactatagaaaaaggccatgtagaagataaaagttcctctataatcccggacccctaagataactactaatgacaacttcatttatattccttcagacattttctggctgtggatgtactaaaatgtatcctattattctctgccctaaaatggaatcatacaaggtgtactgttatttttatggctctataacatgtcatattgtacgtgttggtatggtcattttaaccatttttctagtgatggctttgaggttatttgcagtttcctagccatctcaaagtgtgctgcggggatctcttttgcatccctctgggtgcagagctgaggcacccagaggcagtgtccagaggaggcagcatctgtaggtgtcttcacctgctctggctcttggcacatctggttggtgacactgttttgtgagatgggttgaaagcacgtgctgccaaaatagaataatgttggtcctctcctcatgtgccgtggaactggggtaaaactgcgtagtggctgcagctgcctgtccataccggaatcgagtataacacggtgcctggcttagcacaaaacagtagtgggtcctgcaggccccagagtctaattcctggtattctttcccctacacagattaaataaaccaaaaacaaactattctaggaaagcgtctgtgacatttgtaaaaagtggtatttaatgatcttttattcacttgtctgtttagtttgttgaaatcttaagtggcatcctggtctgggaaggagtgctgtctgcgcctgccctccgctgggcacagcgtggctgcttcaggggctaagcacacactttctgtcttctaaagggccgccacatgccaggagctcaggtgtgagcccggctctggctcttacctcatagggtcactcataggggcacagggagcagaacattgtacacagcgaggcaccacccggcttggcatctgcctcggtggacttactacctctagaaggaaatacctgagttcctctggcctcagctcctagagtgactggtgtgctgtccctgttactcttctgtcaaggtgacaactgtgtgacccatcatctgtgtgtcaaagcaaggccctgcctgggcctctgctcctgtgctgaccccaaaggcaaatgctttgctagtttccttccagttaatttcacctatgaatagatgtgtgaaaactgttcaaagccatacctgcacatgtttgaacttcaaaccctgtgggtgattcagtggcatctttctctaacccccagcctcccttcccacagaggccaccgtcatggccagttgctgcagtttctttccagagaacctgtgtatgtgtaaagctgtacaggcgtgggtacaccacacagcctgtcttgcactgtggactgttgagttactagtacatctaggtaagcaccgcatatctgtattcatgtctgccttggtcttttcaacatctgtgtggtagccgtgtttgaattacccattccctttttggggaaccattaagttgtttcagcaatttttactgtagataaggctataccgcatatctgtgtacatgggtttttatgtacatgggcaagtatatctgtgagagaaaagtttcctcaggaggaattctgggcacagcatgtgtaaatttctaaatatgatggacacccccagcttccacctcaaggaggttggtcccattgacatttccccacaccttcacccaggctgtgcccttaaacttggttatttgtcaatgtgagaagtggaaaatagtatttaattgtagtttggatttgtatttctattgggttgtatacttactgattaataataagagctctttacatattaaggaaattaacccttttcaaatacattcctatttctcactaatctttaagttttattgtaatattttgctctttagtttatatatatatgtatatatatatatatgtatatatatatatatacatatatatatatatatatatatatatatatatatatatatatatatatatatatatatatatatatacatatatatatacatatatatatactaattttcttttatggttcctggattttgtgagtagtttgaaaaggctaatccagctgaagattttgttgttgttgttaaaccccatgttttctcctaactctttttatttttattttggaggactctatctagacttaattttagcataacaagtgacagggttagttagcctgttgtccttacaccattttctggctaatacagctattaactattgatctgtctattcacgtgccagttcctaatggttttacatagtgtaatctgcacttcaaaatagcgaagggaagccctacctcattattctacttttccagaattctcctggctattccaggctgcatgtttaccttaaccttccctgtgatgtcttcatgccgttgtcttcttatgcaagaataaggtacgtctttccatccactcacgtctatttaatttgactttgcattacacagaaagctggtcttggtctgtctacctcggcatctagttgtcctcactgccccctagccgaccccaccccatctgactgactaccccatcacagagtacttttatttacgttttgctctgcctaatggttacttgatactgtcacgccgacagtgtccagttcagtggtctttgcagttgaaatgctcccgtacacactgtcttgttaaaaatgccagtaagttcatacaaacccagcttgcacccaaggtcacattcagagagcgtagggctgggatgggttgttttccaagcttctgccactgtgtggctagctcttcccactgggaagttctgtgtacccggaatgtcggagtggagtcctgttctagtgtccagcacctgaccctgtgcccaacccctcaacagcctattcctgctgtccacagcctgctggaactttttacaaaatatgttgccatgctggaccctgggcactggacataagccccctggcagcctttttcatgtcacccaaaggggtaattgtcctactggtggtctgtaagatgagttagggtgacttgctaatagacattgtaaatcttaatatttatgtatgtattttattattaccggttttccatttatgatggtaatattgtttcttctaagaatatttatttttccttctaaatattgagataaaattcatgcttttgaaatgttctattcagtggcttttagtatatttgctatgttgtgcaaccatcgacactatccatttctagaactttttcgtcatcccaaacagacgctctgtattcataaaaaaataacttcctacctgtctctccccctagtctttggtaacctttgttatactggtaaactttgttgtgctctctgtctgtgtgaatttgcctattctaggggcctcatataagtgtaatcatacagtatttgtctttttgggtctgtctgatttcacttagcgggttttcagggttcattcatgttgcagcatataacagtactgcgttcctttttctggctgaataatattccactgtatggatagaccccattttgtttattcacacatcatttggacatttggattatttctggtttttggctattatgaacaatggtgctatgaacagttgcgtacaagtttttgtgtgaacatatgttttcaattctctcattatatacctaggagtagaattactgggtcatatggtaactgtatatttttgaggaactgccaaactattttcccacgtccatgcaccatttcacattcccaccagtaagtaagagggttccaatttctgcgcattcttgccaacactagttattatctgactttctggttataatcattctaatgagtgtgaagtagcctctggtgtcatttggatttgcatttctctgatgagtgatgctatcaagcacctttgctggtgctgttggccatatgtgtatgttccctggagaagtgtctgtgctgagccttggcccactttttaattaggcgtttgtctttttattactgagttgtaagagttctttatatattctggattctagacccttatcagatacatggtttgcaaatattttctcccattctgtgggttgtgttttcactttatcgataatgtccttagacatataataaatttgtattttaaaagtgacttgatttggctgtgcaaggtggctcacgcttgtaatcccagcactttgggagactgaggtgggtggatcatatgaggaggctaggagttcgaggtcagcctggccagcatagcgaaaacttgtctctactaaaaatacaaaaattagtcaggcatggtggtgcacgtctgtaataccagcttctcaggaggctgaggcacgaggatcacttgaacccaggaggaggaggttgcagtgagctgagatcatgccagggcaacagaatgagactttgtttaaaaaaaaaaaaaa

SEQ ID NO: 89 - DNA, 1821 bp, Homo sapiens
aatgaggccagctggactacgccgagacaactgggagaggcgcgggactcgcccgttccgcggaacgccgggaaggggtcacctcctgatgaagtttccggttccggtgtcagcggcggttgaattgccatggcaatgcggtgggcgcgcgcttgtcgtgttggtctcttgggaggtagtggggctaggccgggcgggtatccgcctctcccagcttaggtgagcgtccccgggcgcctccggagcgccgcggccgcatgcagttcgtcgtggcggggagccggagcctgaccggggttccagcgctcgggccgtagccttggctcctggactttccctggctccgccgccacgtgggagctgaggctctggggcttccgcctccggcgcgcgattatttctctagaacagttttcatttttaaaatttgtaaagcgcttttgcctgtgtgatttcctctgggtttttttttttttttcttcctttttgtagagacggaattggcggcgggggcggggggtcgatgtctcacttttttgcccaggctggtctcgaactcctggcttcaagggatcctcctgcctcggcctcttaaagtgctgggattacaggcgtgagccaccgcccccggccgcctctgagtttccagcctcgttggccctccagccttttaacctgttgggcctaggatcaggaaaggtttgttgaatggggaactaagaagtgaattcgttcgttcgacaaacgtttcctgagcagccgctgggtgctaggcgcagtgccagcgcggaatgtccagggagacctggtgcccaaagcttggacccatcgtgagaaatgagaagcagatacaaagcagtgtgggagtgcagaggagacaaagcaagcctcatcaggcccattgcttgctctgctctcccttgtacttaccagtgcttgacaatatacagttatttactagcttggttattgacttcctatccagcactcagttttattcactgctgtatcctcagtgcctaggacgatgcttggaacgtggtaagtgctcctattggcgggaagaataaatccggaagagcaggaccagtggacttgctacataatctgtagtcttggagccgcacagggttggtggtaccctcgagcacaccagacttgcagaaaaagcatactccagaggaagctgaggcatgcctgctcgagagccagctgttccatgtgcaattttcctctgatagtttctggtcactgttgccacggtgataatgactgggctatgtcattatctatccgccaacagtaagagaagctttgcagtcgagatattgtttagcagatggagtgttttctgttgaacactaagtactgccacaagttactttttttttttttaaactttgagtatttttttacaatgttgctggaggtgatctgtttatgctttgagagtgttcgaatttaaaatcagaaaatcatgtcagtgagtgagtctttcaaataatccttcggcatgaaacctgagcctagtaaactatgaaagtaaactcggcacattacccgaaagtctcaatgtcatattttcacccccatcaatattattgatgattgctcattttctaatgtgggacctgaaatttaccaggtgcttaaagaatctttttgtttttcagattcattgattccaggtaaatcagaggaacaagcaacatgaacagaaatatgtagaaaaagctattatgcagaagcataattgttgtttcagaagtccagcatctggtgcacttaacaatagagaatatattaaactctttccaaaat

SEQ ID NO: 90 - DNA, 2856 bp, Homo sapiens; identical to SEQ ID NO: 4

SEQ ID NO: 91 - DNA, 920 bp, Homo sapiens
gcacggaggggcagagaccccggagccccagccccaccatgaccctcggccgccgactcgcgtgtcttttcctcgcctgtgtcctgccggccttgctgctggggggcaccgcgctggcctcggagattgtggggggccggcgagcgcggccccacgcgtggcccttcatggtgtccctgcagctgcgcggaggccacttctgcggcgccaccctgattgcgcccaacttcgtcatgtcggccgcgcactgcgtggcgaatgtaaacgtccgcgcggtgcgggtggtcctgggagcccataacctctcgcggcgggagcccacccggcaggtgttcgccgtgcagcgcatcttcgaaaacggctacgaccccgtaaacttgctcaacgacatcgtgattctccagctcaacgggtcggccaccatcaacgccaacgtgcaggtggcccagctgccggctcagggacgccgcctgggcaacggggtgcagtgcctggccatgggctggggccttctgggcaggaaccgtgggatcgccagcgtcctgcaggagctcaacgtgacggtggtgacgtccctctgccgtcgcagcaacgtctgcactctcgtgaggggccggcaggccggcgtctgtttcggggactccggcagccccttggtctgcaacgggctaatccacggaattgcctccttcgtccggggaggctgcgcctcagggctctaccccgatgcctttgccccggtggcacagtttgtaaactggatcgactctatcatccaacgctccgaggacaacccctgtccccacccccgggacccggacccggccagcaggacccactgagaagggctgcccgggtcacctcagctgcccacacccacactctccagcatctggcacaataaacattctctgttttgt

(19) United States
(12) Patent Application Publication
(10) Pub. No.: US 2008/0286763 A1
(43) Pub. Date: Nov. 20, 2008

(54) Method For The Identification Of Sepsis

(76) Inventors: Stefan Russwurm, Jena (DE); Hans-Peter Deigner, Lampertheim (DE)

Correspondence Address: AKERMAN SENTERFITT, P.O. BOX 3188, WEST PALM BEACH, FL, 33402-3188, US

(21) Appl. No.: 10/591,371

(22) PCT Filed: Dec. 15, 2004
(86) PCT No.: PCT/EP04/14310

§ 371 Date: Dec. 8, 2006

(30) Foreign Application Priority Data
Mar. 1, 2004 (DE) 10 2004 009 952.9

(51) Int. Cl. C12Q 1/68; G01N 33/50; G06G 7/58; C40B 30/04

(52) U.S. Cl. 435/6; 436/94; 506/9; 703/11

(57) ABSTRACT

The invention relates to a method for the in vitro discrimination between systemic inflammatory non-infectious conditions and systemic inflammatory infectious conditions. The method comprises the following steps: a) sample RNA is isolated from a biological sample; b) the sample RNA and/or at least one DNA which represents a gene activity that is specific for distinguishing between SIRS and sepsis and/or a specific gene or gene fragment, is marked with a detectable marker; c) the sample RNA is brought in contact with the DNA in hybridization conditions; d) control RNA is brought in contact with at least one DNA in hybridization conditions, the DNA representing a gene or gene fragment that is specific for distinguishing between SIRS and sepsis; e) the marking signals of the hybridized sample RNA and control RNA are quantitatively recorded; and f) the quantitative data of the marking signals is compared in order to make a statement as to whether genes or gene fragments that are specific for distinguishing between SIRS and sepsis are expressed more prominently or less prominently in the sample than in the control RNA.

### CROSS REFERENCE TO RELATED APPLICATION

This application is a national stage of PCT/EP2004/014310 filed Dec. 15, 2004 and based upon DE 10 2004 009 952.9 filed Mar. 1, 2004 under the International Convention.

### BACKGROUND OF THE INVENTION

### Field of the Invention

The present invention relates to a method for the in vitro differentiation between systemic inflammatory non-infectious conditions and systemic inflammatory infectious conditions according to claim 1.

The term “generalized, inflammatory, non-infectious condition” as used in the following corresponds to the definition of SIRS according to [1] and “generalized, inflammatory, infectious condition” corresponds to the definition of sepsis according to [1].

The present invention in particular concerns the use of gene activity markers for the diagnosis of sepsis.

The present invention further relates to new diagnostic possibilities, which can be derived from experimentally verified insights in conjunction with the occurrence of changes in gene activity (transcription) in patients with SIRS and sepsis.

Despite advances in phytopathological understanding and the supportive treatment of intensive care patients, systemic inflammatory conditions such as SIRS and sepsis, defined according to the ACCP/SCCM Consensus Conference of 1992 [1], frequently occur in patients in intensive care facilities and contribute substantially to mortality of infections [2-3]. The mortality is approximately 20% in the case of SIRS, approximately 40% in the case of sepsis, and increases up to 70-80% in the case of development of multiple organ dysfunctions [4-6]. The morbidity and lethality contribution of SIRS and sepsis is of multi-disciplinary clinical-medical significance, since in increasing measure the success of treatment in advanced therapeutic processes of various medical field [for example traumatology, neurosurgery, cardiac/pulmonary surgery, visceral surgery, transplantation, hematology/oncology, etc.] is endangered thereby, since without exception an elevation of the risk of affliction with SIRS and sepsis in imminent. This also finds expression in the continuous increase in the frequency of sepsis: between 1979 and 1987 by 139% from 73.6 to 186 incidents of illness per 100,000 hospital patients [7]. Any suppression in the morbidity and lethality of a number of seriously ill patients is thus associated with a simultaneous advance in the prophylaxis, treatment and in particular the recognition and the monitoring of the progression of sepsis and acute sepsis.

On the molecular plane sepsis is characterized as an illness caused by pathogenic micro-organisms. Following the exhaustion of molecular control and regulation options near to the infection, a generalized, comprehensive organism-encompassing inflammation reaction develops, which is responsible for the doctor verified clinical symptoms/diagnosis criteria/SIRS-criteria according to [1]. This generalized, inflammatory condition (defined as sepsis according to [1]) accompanies indications of activation of various cell systems (endothelial cells, also all leucocytic cell systems and above all the monocytic/macrophage system). Finally, molecular mechanisms, which should actually protect the host against invasive micro-organisms, damage its own organs/tissues, and thus contribute significantly to the development of clinically dreaded organ dysfunctions [8-11].

The concept of sepsis has experienced a substantial evolution in meaning over time. An infection or, as the case may be, an urgent suspicion of an infection, remain today essential components of actual sepsis definitions. Of particular consideration is however the description of organ failure functions remote from the location of infection in the framework of the inflammatory host reaction. In the international literature in the meantime the criteria from the Consensus Conference of the “American College of Chest Physicians/Society of Critical Care Medicine Consensus Conference (ACCP/SCCM)” from 1992 have accepted the broadest definition of the sepsis concept [1]. According to the criteria [1] distinctions are made between the clinically defined degrees of seriousness: “systemic inflammatory response syndrome” (SIRS), “sepsis,” “severe sepsis,” and “septic shock.” SIRS is defined as the systemic response of the inflammatory system to an infectious or non-infectious stimulus. For this, at least two of the following clinical criteria must be satisfied: fever >38° C. or hypothermia <26° C., a leucocytosis >2 G/l or a leucopenia <4 G/l or, as the case may be, a shift to the left in the differential hemogram, a pulse rate of 90/min, a respiration >20 breaths/min or a PaCO₂ (partial pressure of carbon dioxide in arterial blood)<4.3 kPa. Those clinical conditions which satisfy the SIRS criteria and for which causatively an infection can be confirmed or at least is very probable are defined as sepsis. A severe sepsis is characterized by the supplemental occurrence of failure of organ functions. Common organ function failures include changes in orientation or awareness, oliguria, lactacidosis or a sepsis-induced hypotension with a systolic blood pressure of less than 90 mmHg or, as the case may be, a drop in pressure of greater than 40 mmHg from the initial value. If such hypotension cannot be alleviated by the administration of crystal iodides and/or colloids and a catecholamine binding of the patient occurs, then one refers to this as septic shock. This is confirmed in approximately 20% of all sepsis patients.

Sepsis is the clinical result of complex and highly heterogeneous molecular processes, which are characterized by the involvement of many components and their interaction on every organismic level of the human body: genes, cells, tissue, organs. The complexity of the underlying biologic and immunologic processes have spawned many types of research studies, which encompass a large range of clinical aspects. One of the results to be recognized therefrom is that the evaluation of new sepsis therapies is made more difficult by the relatively unspecific, clinical based decision or involvement criteria, which do not sufficiently take into consideration the molecular mechanisms [12]. At the same time, due to the lack of specificity of the prevailing sepsis and SIRS diagnosis among the clinicians, large differences exist, beginning with the point in time at which a patient is to be administered a specialized therapy, for example antibiotics, which for their part can have substantial side effects [12]. A survey carried out of the European Society of Intensive Care Medicine (ESICM) showed that 71% of the questioned doctors were unsure regarding the diagnosing of sepsis, despite many years of clinical experience [22].

Ground-breaking discoveries in molecular biology and immunology during the last two decades have allowed the development of a deeper understanding of sepsis oriented more towards the basic mechanisms. The knowledge resulting therefrom regarding relevant targets has formed the basis for the discovery of target and adjuvant therapeutic concepts, which primarily concern the neutralization of essential sepsis mediators [13-16]. One cause for the failure of almost all immuno-modulator type therapies in clinical studies—despite affectivity in animal experiments—is considered to be the poor correlation between the clinical, primarily symptomatic oriented diagnostic criteria and the fundamental mechanisms of the systemic immuno-response [12, 17-18].

In retrospect it is not surprising that even healthy humans, in the course of their daily activities, experience fluctuations in cardiac or, as the case may be, respiratory frequencies, which fall within the definition of the diagnosis of SIRS. In consideration of our current bio-medical possibilities it must appear to be an anachronism, that annually 751,000 patients in the USA are diagnosed, classified and treated on the basis of the above-discussed ACCP/SCCM criteria. Prominent authors thus have already long criticized that, at the expense of an improved sepsis diagnosis, in the past decade too much energy and financial resources have been lavished on the search for a “magic bullet” for sepsis therapy [19]. Recently published experiments also indicate that for a better pathophysiologic understanding of sepsis a modification of the consensus criteria according to [1] is necessary [21-21]. Besides this, there is consensus among many medical practitioners that the consensus criteria according to [1] do not provide any specific definition of sepsis. Similarly, a survey of the European Society of Intensive Care Medicine (ESICM) shows that 71% of the questioned doctors were uncertain regarding diagnosis of sepsis, despite many years of clinical experience [22].

As a result of the above mentioned problems with the application of the consensus criteria according to [1], proposals are being discussed among intensive care medical practitioners for a sensitive and specific definition of the various degrees of seriousness of sepsis [2, 23]. What is new therein above all is that molecular changes are to be directly taken into consideration in the evaluation of the seriousness of sepsis, and further the inclusion of innovative treatment processes of sepsis (such as for example the therapy with activated recombinant protein C). These consensus process [23], which currently is being carried out by five international professional associations, is at the current time long from completion. The goal is the establishment of a system for judging the seriousness of sepsis, which makes it possible to classify patients on the basis of their individual patient reaction on the basis of their predispositioning conditions, the type and the magnitude of the infection, the type and the seriousness of the host reaction as well as the degree of the accompanying organ dysfunctions. The described symptomology is referred to PIRO, abbreviated to the English terms for “predisposition,” “insult infection,” “response,” and “organ dysfunction.” From this, the individual probability of the survival as well as the potential response to the therapy can be derived [23]. At the same time non-infectious conditions, which are currently subsumed under the concept of SIRS according to [1], corresponding to the individual seriousness of the SIRS can be more precisely classified. For this also bio-markers are sought, which reflect the seriousness of the SIRS on a molecular plane and which enable a clear delineation of infectious conditions (currently classified as sepsis in accordance with [1]). Similar phase introductions are already used today by other medical specialists with success, for example for classification of the different degrees of illness in the field of oncology (TNM System, [24]).

One important criteria for the diagnosis of sepsis is, besides the systemic inflammatory reaction, the evidence of infection. From [25] its is however known that, for example, from approximately 8500 blood cultures from one division of internal medicine, only in approximately 15% of all blood cultures could the pathogen or causative organism be identified. For blood cultures of an anesthesialogical intensive care station evaluated over the same period of time (I year), only in approximately 10% of all blood cultures could the cause of the infection be identified. This research proves the problem of finding an early proof of infection and therewith an early diagnosis of sepsis. As cause for the lack of identification of causative agent using blood cultures, it may be the lack of consensus of methods of culturing special pathogens in general, as well as the frequent simultaneous implementation of antibiotic therapy in particular, which leads thereto, that the pathogen is no longer metabolically active and thus non-proliferating. Compared with the consensus criteria according to [1], in the future supplemental molecular parameters are to be taken into consideration in the establishment of the diagnosis [23], in order to make possible an improved correlation of the molecular inflammatory/immunological host response to the degree of the sepsis. At the present time various professional and commercial groups are intensively searching for such molecular biomarkers, since a conventional parameter such as, for example, the determination of the C-reactive protein or the procalcitonins do not satisfy all clinical requirements [26]. Also, do the insufficient specificity and sensitivity of the consensus criteria according to [1] at the lack of or delay of proof of the cause of the infection, there exists thus an urgent requirement for a new diagnostic processes, which improves the ability of the professional to provide an early diagnostication of sepsis, to do this reproducibly in the clinical setting, and to allow conclusions to be derived with regard to the individual prognosis and the response to specific treatments.

Technological advances, in particular the development of the micro-array technology, make it possible for the clinician now to simultaneously compare 10,000 or more genes and their gene products. The use of such microarray technologies can now provide information regarding the status of health, regulatory mechanisms, biochemical interactions and signal transmitter networks. The improvement in the understanding regarding how an organism reacts to infections should facilitate the development of stronger detection, diagnosis and treatment modalities for sepsis afflictions.

Microarrays are derived from “Southern Blotting” [27], which represented the first approach to immobilizing DNA-molecules in a spatially acceptable mode and manner on a solid matrix. The first microarrays were comprised of DNA-fragments, often with unknown sequence, and were spotted on a porous membrane (normally nylon) routinely cDNA, genomic DNA or plasmid libraries were employed, in order to mark the hybridized material with a radioactive group [28-30].

Recently the use of glass as substrate and fluorescence for detection together with the development of new technologies for synthesis and for the application of nucleic acids in a very high density made it possible to miniaturize the nucleic acid arrays with simultaneous elevation of the experimental through-put and information content [31-33].

Further it is known from WO 03/002763 that microarrays could in principle be used for the diagnosis for sepsis and sepsis-like conditions.

A justification of the applicability of microarray technology was first provided by clinical research in the field of cancer research. Here, expression profiles have demonstrated their utility in the identification of activities of individual genes or gene groups, which correlate with a certain clinical phenotypes [34]. By the analysis of many samples, which originated from individuals with or without acute leucamine or defused B-cell lymphomas, gene expression markers (RNA) were found and subsequently employed for the clinically relevant classification of these types of cancers [34, 35]. Golub et al. have discovered that reliable predictions could not be made on the basis of any individual gene, while however predictions based on the change of the transcription of 53 genes (selected from more than 6000 genes, which were represented on the arrays) were very precise [34].

Alisadeh et al. [35] researched large B-cell lymphomas (DLBCL). The expression profile was processed or worked up by the authors with a “lymphochip”, which carried 18,000 clone complimentary DNA and was developed to monitor genes which are involved in normal and abnormal lymphocyte development. With use of cluster analysis they were able to separate DILBCL into two categories, which indicated strong differences with regard to the survival chances of the patients. The gene expression profiles of these subgroups correlated to two significant changes of the B-cell differentiation.

In the field of neurology a large number of studies were carried out for identification of gene active markers by means of microarray technology [36]. The same applied for the research of molecular changes, which could be triggered by individual components of bacterial Gram negative pathogens (for example using stimulation experiments with lipopolysaccharides) [37]. Such research was as a rule carried out by means of the cellular model system, for example human endothelial cell cultures [38], or in human leucocytic cell cultures [41], or also for example by means of research of human tissue but however not blood in [39]. Therein the orientation of the experimental goal was concerned with identification of until now unknown participants of the cellular signal transmission pathways, in order in this manner to be able to better to describe the molecular nature of an inflammation. Alternatively, for such problems animal experiments, for example with mice, were also routinely carried out [40].

A further example of the use of the differential gene expression for deeper research into the molecular processes in a generalized inflammation reaction could be shown on the basis of cDNA based microarrays in [42].

The measurement of gene expression profiles for distinguishing between SIRS according to [1] and sepsis according to [1] was not described.

The starting point for the invention disclosed in the present patent application is the recognition that gene activity of various genes in biological samples of an individual, in which sepsis-typical indications (corresponding to the definition [1]) have been confirmed, can be distinguished from the gene activity of the same genes in samples of individuals which have been diagnosed with SIRS. This distinction in the gene activity thus enables distinction between patients with sepsis, that is, a supplemental infectious complication, from patients without this infectious complication (SIRS according to [1]). As already explained in another location, this distinction has until now been associated with substantial disadvantages, however is very significant for the introduction of a specialized medical therapy and therewith for the improvement of the individual prognosis for survival.

The present invention is thus concerned with the task of providing a process which makes possible the distinction between systemic, inflammatory, non-infectious conditions (SIRS according to [1]) and systemic, inflammatory, infectious conditions (sepsis according to [1]).

This task is solved by a process with the characterizing features of claim 1.

The invention is further concerned with the task of providing a possibility of use of markers in a process according to claim 1-25.

This task is solved by the use according to claims 26-32.

The inventive process is characterized thereby, that in a sample of a biological fluid from an individual the activity of one or more marker genes is determined, and from the determined presence and/or amount of the identified gene product distinction can be made between SIRS and sepsis (both according to [1]).

The carrying out of the invention is characterized thereby, that the process for in vivo distinguishing between SIRS and sepsis comprises the following steps:

a) isolation of sample RNA from a biological sample;

b) marking the sample RNA and/or at least one DNA which represents a gene activity that is specific for distinguishing between SIRS and sepsis (both according to [1]) and/or a specific gene or gene fragment, with a detectable marker; bringing the sample RNA in contact with the DNA in hybridization conditions; bringing control RNA in contact with at least one DNA in hybridization conditions, said DNA representing a gene or gene fragment that is specific for distinguishing between SIRS and sepsis;

c) quantitatively measuring the marking signals of the hybridized sample RNA and control RNA; and

d) comparing the quantitative data of the marking signals in order to make a determination as to whether genes or gene fragments that are specific for distinguishing between SIRS and sepsis (both according to [1]) are expressed more prominently or less prominently in the sample than in the control RNA.

A further embodiment of the invention is characterized thereby, that the control-RNA, prior to measurement of the sample-RNA, is hybridized with the DNA, and the marker signals of the control-RNA/DNA-complex are determined and in certain cases recorded in the form of a calibration curve or table.

A further embodiment of the invention is characterized thereby, that mRNA is used as the sample-RNA.

A further embodiment of the invention is characterized thereby, that the DNA is placed in predetermined areas on a carrier in the form of a microarray, and, in particular, is immobilized.

A further embodiment of the invention is characterized thereby, that the process is employed for differential diagnostic early recognition, for control of the therapeutic procedure, and for risk assessment for patients.

A further embodiment of the invention is characterized thereby, that the sample is selected from: body fluids, in particular blood, serum, urine, peritoneal fluid, seminal fluid, saliva, tissue fluid; cellular contents, or a mixture thereof.

A further embodiment of the invention is characterized thereby, that cell samples are in certain cases subject to lytic treatment, in order to release their cellular contents.

A further embodiment of the invention is characterized thereby, that the biological sample is of human origin.

A further embodiment of the invention is characterized thereby, that for researching SIRS and sepsis, specific genes or gene fragments are selected from the group comprising SEQ-ID No. 1 through SEQ-ID No. 91, as well as fragments thereof with at least 5-2,000, preferably 20-200, more preferably 20-80 nucleotides.

These sequences with a sequence ID: 1 through sequence ID: 91 are included within the scope of the present invention and are disclosed in detail in the attached 42-page, 91-sequence-covering sequence protocol which therewith becomes part of the invention. This sequence protocol includes, besides this, classification or correlation of the individual sequences with the sequence ID: 1 through sequence ID: 91 to their GeneBank Accession No. (Internet access via www.ncbi.nim.nih.gov/).

A further embodiment of the invention is characterized thereby, that the immobilized or free samples are marked. For this embodiment, self complimentary oligonucleotides, so called molecular beacons, are used as the testing samples. They carry a fluorophor/quencher-pair on their ends, so that in the presence of a complimentary sequence they are present in the form of their folded hairpin structure and provide a fluorescence signal only with a corresponding sample sequence. The hairpin structure of the molecular beacon is stable until the sample is hybridized on the specific capturing sequence, which leads to a conformation change and therewith to release of the reporter fluorescence.

A further embodiment of the invention is characterized thereby, that at least 2 to 100 cDNAs are employed.

A further embodiment of the invention is characterized thereby, that at least 200 cDNAs are employed.

A further embodiment of the invention is characterized thereby, that at least 200 to 500 cDNAs are employed.

A further embodiment of the invention is characterized thereby, that at least 500 to 1000 cDNAs are employed.

A further embodiment of the invention is characterized thereby, that at least 1000 to 2000 cDNAs are employed.

A further embodiment of the invention is characterized thereby, that as the DNA the genes listed in claim 10 are replaced by their RNA derived sequences, synthetic analogs, aptamers as well as peptido-nucleic acids.

A further embodiment of the invention is characterized thereby, that the synthetic analogs of the genes include 5-100, in particular approximately 70 base pairs.

A further embodiment of the invention is characterized thereby, that as the detectable markers a radioactive marker, in particular ³²P, ¹⁴C, ¹²⁵I, ¹⁵⁵Ep, ³³P, or ³H is employed.

A further embodiment of the invention is characterized thereby, that as detectable markers a non-radioactive marker, in particular a color or fluorescence marker, an enzyme or immuno marker and/or quantum dots or an electrically measurable signal, in particular potential/potentiometric and/or conductivity and/or capacitance changes during hybridization are employed.

A further embodiment of the invention is characterized thereby, that the sample RNA and control-RNA and/or enzymatic or chemical derivatives thereof carry markers.

A further embodiment of the invention is characterized thereby, that the sample RNA and control-RNA and/or enzymatic or chemical derivatives carry different markers.

A further embodiment of the invention is characterized thereby, that the DNA-samples are immobilized on glass or plastic.

A further embodiment of the invention is characterized thereby, that the individual DNA molecules are immobilized via their covalent bond to the carrier material.

A further embodiment of the invention is characterized thereby, that the individual DNA molecules are immobilized by means of electrostatic and/or dipol-dipol and/or hydrophobic interaction and/or hydrogen bonds to the carrier material.

A further embodiment of the invention is comprised in the use of recombinant or synthetic produced specific nucleic acid sequences for distinguishing between SIRS and sepsis (both according to [1]), partial sequences individually or in partial amounts as calibrators in sepsis assays and/or for evaluation of effectiveness and toxicity in active substance screening and/or for production of therapeutics and materials and material mixtures which are provided as therapeutics for prevention and treatment of SIRS and sepsis.

It is apparent to the person skilled in the art that the individual characteristics of the invention set forth in the claims can be combined with each other without limitation.

As marker genes in the sense of the invention all derived DNA-sequences, partial sequences and synthetic analogs (for example peptido-nucleic acids, PNA) are included. For the determination of the gene expression with regard to the description of the invention on the RNA-level, no limitation is implied, but rather this is to be considered only an exemplary use.

The description of the invention with respect to blood represents only one exemplary embodiment of the invention. The biologic fluids could, in the sense of the invention, be any body fluids of the human.

An application of the inventive process is in the measurement of the differential gene expression for distinguishing between SIRS and sepsis (both in accordance with [1]). For this, the RNA is isolated from the whole blood of appropriate patients and a control group of healthy test persons or non-infectious patients. The RNA is subsequently marked, for example radioactively with ³²P or with color molecules (fluorescence). As marker molecule all molecules known for this purpose in the state of the art and/or detection signals can be employed. Corresponding molecules and/or processes are likewise known to the person of ordinary skill in the art.

The thus marked RNA is subsequently hybridized on a microarray of immobilized DNA-molecules. The DNA molecules immobilized on the microarray provide a sufficient selection of genes according to claim 10 of the present invention for distinguishing SIRS and sepsis.

The intensity signal of the hybridized molecules are measured in conjunction with suitable measuring devices (phosphorescent images, microarray scanners) and analyzed by additional software supported evaluations. From the measured signal intensities the expression relationship between the patient sample and the control sample is determined. From the expression relationships of the under and/or over regulated genes, conclusions can be made, as shown in the following experiments, with regard to distinguishing between SIRS and sepsis.

A further application of the inventive process is comprised in the measurement of the differential gene expression for therapy accompanying determination of the probability whether patients would respond to the planned therapy, and/or for the determination of the response to the specialized therapy and/or on the determination of the therapy termination in the sense of a “drug monitoring” in patients with SIRS and sepsis. For this, the RNA (sample RNA) is isolated from blood samples collected over time from the patients. The different RNA samples are marked together with the control sample and are hybridized with selected genes according to claim 10, which are immobilized on a microarray. From the respective expression relationships it can be evaluated or determined which probability there is that patients would respond to a planned therapy and/or whether the initiated therapy would be effective and/or the length to which patients still need to be subject to therapy and/or whether the maximal therapeutic effect has already been achieved with the employed dosing and duration.

A further application of the inventive process is comprised in the use of the RNA of the gene according to claim 10 for obtaining quantitative information by hybridization-independent processes, in particular enzymatic or chemical hydrolysis, subsequent quantification of the nucleic acids and/or derivatives and/or fragments of the same.

A further application of the inventive process is comprised in the use of the gene activity for distinguishing SIRS and sepsis for the electronic further processing for purpose of production of software for diagnostic purposes (for example for patient data management systems), or experiment systems for modeling for cellular signal transmitter pathways or for purposes of computer supported modeling of inflammatory conditions also in model organisms such as for example C. elegans or Saccharomyces cerevisiae.

Further advantages and characteristics of the invention can be seen from the description of the example.

### ILLUSTRATIVE EXAMPLE

Research into differential gene expression for distinguishing between systemic, inflammatory, non-infectious conditions (corresponding to SIRS in accordance to [1]) and systemic, inflammatory, infectious conditions (corresponding to sepsis in accordance to [1]).

For the measurement of differential gene expression for distinguishing SIRS and sepsis analysis of whole blood samples of patients, which were treated in an operating intensive care station, was carried out.

Whole blood samples of five male and one female patient were obtained (patient samples). Each of these patients developed sepsis in the framework of their intensive medical intervention following a by-pass operation. The patient samples were drawn immediately (within 12 hours) after initial diagnosis of sepsis corresponding to the classification according to [1] was obtained. Selective characteristics of the patients with sepsis are indicated in Table 1. Therein, indications regarding age, sex, cause of sepsis (see diagnosis as well as clinical acuteness), measured according to—in clinical literature well documented—APACHE-II- and SPFA-Scores (respectively in Dots) was made. Likewise the plasma protein level of procalcitonin (PCT), a more recent sepsis marker, the Center for Disease (CDC)-criteria (see http://www.cdc.gov) and the individual survival status are indicated.

As control samples whole blood samples of the same patients were used. These were respectively collected one-day postoperative. At this point in time each had an operation associated SIRS defined according to [1] (on the basis of employment of the heart-lung machine).

TABLE 1
Data for the Patient Group

| | | | | | | APACHE-II | SOFA | | | |
|---|---|---|---|---|---|---|---|---|---|---|
| | | | | | Classification | Score | Score | PCT | | |
| Patient | Age | Sex | Sample | Diagnosis | per [1] | [Points] | [Points] | [ng/ml] | CDC-Criteria | Mortality |
| Patient 1 | 60 | male | Control | 3-Vascular-KHK | SIRS | 9 | 6 | 5.38 | Pneumonia | Survived |
| | | | Sample | | Sepsis | | 11 | 13.1 | | |
| Patient 2 | 80 | female | Control | Aortic flap stenosis | SIRS | 14 | 8 | 2.09 | Pneumonia | Died |
| | | | Sample | | Sepsis | | 8 | 3.81 | | |
| Patient 3 | 76 | male | Control | Mitral flap failure | SIRS | 15 | 9 | 9.11 | Pneumonia | Survived |
| | | | Sample | | Sepsis | | 10 | 1.2 | | |
| Patient 4 | 61 | male | Control | Mitral flap stenosis | SIRS | 11 | 12 | 14.5 | Intra-abdominal | Died |
| | | | Sample | | Sepsis | | 21 | 44 | infection | |
| Patient 5 | 63 | male | Control | Atherosclerotic heart | SIRS | 12 | 11 | 1.23 | Focus unclear | Died |
| | | | Sample | disease | Sepsis | | 14 | 3.64 | | |
| Patient 6 | 65 | male | Control | Atherosclerotic heart | SIRS | 16 | 8 | 4.22 | Pneumonia | Survived * |
| | | | Sample | disease | Sepsis | | 5 | 0.3 | | |

After drawing whole blood, the total RNA was isolated using an PAXGene Blood RNA kit according to the directions of the manufacturer (Qiagen). Subsequently, from the total RNA, the double stranded cDNA was synthesized by means of reverse transcription using the Agilent Low RNA Input Fluorescent Amplification Kit (Agilent) according to the protocol provided by the manufacturer, wherein a T7 RNA polymerase-promoter was attached on the Poly-A and of the cDNA. Subsequently the cDNA was synthesized using the T7 RNA polymerase promoter and simultaneous insertion of fluorescence nucleotides Cy3/Cy5-Cytosintriphosphate (Amersham) in cRNA, which served as hybridization molecules. All RNA-samples were divided into two aliquots, of which one aliquot was marked with Cy3-CTP and the other aliquot with Cy5-CTP. Thereby each co-hybridization could be carried out twice with use of the reversed RNA/fluorescence dye combination.

Each of the prepared combinations of the hybridization molecules was hybridized both with the Microarray 1A Oligo as well as with the 1B Oligo from the manufacturer Agilent according to the protocol of the manufacturer. Together these two microarrays contain 36,000 genes and ESTs (Expressed Sequence Tags). The fluorescence signals of the hybridized molecules were measured using a reader device (Agilent DNA Microarray Scanner) and computed using the software Agilent Feature Software.

### Evaluation

For evaluation, the average intensity of one spot was determined as the median value associated with the spot pixel.

### Correction of Systemic Errors:

The median of the pixel of the local background was subtracted from the median of the spot pixel. For all further computations the signal was transformed by means of arcus sinus hyperbolicus. The normalization occurred according to the approach of Huber et al. [43]. Therein the additive and the multiplicative bias within a microarray was estimated from 70% of the present gene sample. The intensity signals from the red channel were then corrected.

### Statistical Comparison

For comparison the paired student test was employed. The test was carried out independently for both experimental conditions. For the selection of the differentiated experimental genes, the associated p-value and the average expression change within the sample was evaluated.

### Results

It applied for the group of deselected genes that in both experiments the associated p-value was smaller than 0.05 and the average expression change was greater than 1.2.

The magnitude of the expression relationship of each gene provided the criteria for a sorting of the examined genes. Of interest were the genes which were most over-expressed or, as the case may be, under-expressed in the patient samples relative to the control samples.

It can be seen from Table 2 that 51 genes of the patient samples were found which were significantly over-expressed in the patient samples relative to the control samples. Further, it is clear from Table 3 that 17 genes of the patient samples were significantly under-expressed in the patient samples relative to the control samples. From the results it is clear that the gene activities listed in Table 2 and Table 3 distinguish between systemic, inflammatory, infectious conditions (corresponding to sepsis according to [1]) and systemic, inflammatory, non-infectious conditions (corresponding to SIRS according to [1]). Therewith the listed gene activities provide markers for the distinguishing between SIRS and sepsis.

Table 2: Significant elevated gene activities in samples of patients with sepsis according to [1], indicated as their relative relationship to the corresponding gene activity of the same patient in the condition of SIRS according to [1].

TABLE 2

| GenBank Acc. | | mean: | mean: | p: | p: | |
|---|---|---|---|---|---|---|
| Number | HUGO-Name | Cy5 vs Cy3 | Cy3 vs Cy5 | Cy5 vs Cy3 | Cy3 vs Cy5 | Seq.-ID |
| NM_006986.2 | MAGED1 | 1.33 | 1.36 | 0.01 | 0.01 | 1 |
| NM_005319.1 | H1F2 | 1.21 | 1.09 | 0.01 | 0.01 | 2 |
| NM_001925.1 | DEFA4 | 1.16 | 1.26 | 0.00 | 0.00 | 3 |
| NM_006516.1 | SLC2A1 | 1.02 | 0.84 | 0.02 | 0.02 | 4 |
| D87452.1 | IHPK1 | 0.97 | 0.88 | 0.01 | 0.01 | 5 |
| NM_020070.1 | IGLL1 | 0.97 | 0.98 | 0.02 | 0.01 | 6 |
| NM_022771.1 | FLJ12085 | 0.97 | 0.90 | 0.00 | 0.00 | 7 |
| NM_001738.1 | CA1 | 0.88 | 0.89 | 0.00 | 0.00 | 9 |
| L05148.1 | ZAP70 | 0.82 | 0.74 | 0.02 | 0.01 | 10 |
| BC021275.1 | FLJ32987 | 0.68 | 0.65 | 0.03 | 0.01 | 13 |
| NM_005321.1 | H1F4 | 0.65 | 0.61 | 0.01 | 0.01 | 15 |
| NM_005564.1 | LCN2 | 0.58 | 0.60 | 0.01 | 0.00 | 17 |
| NM_003250.1 | THRA | 0.56 | 0.45 | 0.04 | 0.02 | 18 |
| NM_005067.1 | SIAH2 | 0.54 | 0.54 | 0.00 | 0.00 | 19 |
| NM_016417.1 | LOC51218 | 0.49 | 0.30 | 0.01 | 0.04 | 21 |
| NM_005764.1 | DD96 | 0.47 | 0.60 | 0.04 | 0.01 | 22 |
| NM_033445.1 | H2AFA | 0.46 | 0.40 | 0.00 | 0.04 | 23 |
| M18728.1 | CEACAM6 | 0.45 | 0.29 | 0.01 | 0.03 | 24 |
| NM_003516.1 | H2AFO | 0.43 | 0.47 | 0.05 | 0.05 | 27 |
| NM_018639.1 | LOC55884 | 0.43 | 0.28 | 0.04 | 0.04 | 28 |
| BC029812.1 | ZNF145 | 0.40 | 0.27 | 0.02 | 0.02 | 29 |
| NM_021052.1 | H2AFA | 0.39 | 0.42 | 0.04 | 0.04 | 30 |
| NM_001911.1 | CTSG | 0.39 | 0.42 | 0.02 | 0.01 | 31 |
| NM_005907.1 | MAN1A1 | 0.38 | 0.28 | 0.01 | 0.05 | 32 |
| NM_003523.1 | H2BFH | 0.37 | 0.32 | 0.04 | 0.05 | 33 |
| NM_015523.1 | DKFZP566E144 | 0.37 | 0.29 | 0.01 | 0.01 | 34 |
| NM_003527.4 | H2BFN | 0.37 | 0.32 | 0.03 | 0.04 | 35 |
| NM_015277.1 | NEDD4L | 0.34 | 0.32 | 0.00 | 0.00 | 36 |
| NM_000250.1 | MPO | 0.33 | 0.30 | 0.01 | 0.02 | 37 |
| NM_015972.1 | LOC51082 | 0.33 | 0.31 | 0.04 | 0.03 | 39 |
| NM_021063.1 | H2BFB | 0.33 | 0.38 | 0.05 | 0.02 | 39 |
| NM_017802.1 | FLJ20397 | 0.32 | 0.33 | 0.03 | 0.04 | 40 |
| NM_003258.1 | TK1 | 0.32 | 0.37 | 0.04 | 0.03 | 41 |
| NM_003514.2 | H2AFN | 0.31 | 0.30 | 0.02 | 0.01 | 43 |
| NM_031894.1 | FTHL17 | 0.29 | 0.33 | 0.04 | 0.03 | 44 |
| AJ296290.1 | PRKWNK1 | 0.29 | 0.32 | 0.01 | 0.01 | 45 |
| NM_016614.1 | AD022 | 0.28 | 0.21 | 0.00 | 0.04 | 47 |
| NM_021064.2 | H2AFP | 0.26 | 0.29 | 0.03 | 0.04 | 48 |
| NM_006563.1 | KLF1 | 0.26 | 0.39 | 0.01 | 0.01 | 49 |
| NM_004617.1 | TM4SF4 | 0.25 | 0.22 | 0.00 | 0.00 | 50 |
| NM_006875.1 | PIM2 | 0.25 | 0.25 | 0.04 | 0.05 | 51 |
| NM_016068.1 | LOC51024 | 0.24 | 0.33 | 0.03 | 0.01 | 52 |
| NM_002466.1 | MYBL2 | 0.24 | 0.34 | 0.04 | 0.01 | 53 |
| NM_021014.1 | SSX3 | 0.24 | 0.41 | 0.00 | 0.00 | 54 |
| NM_003779.2 | B4GALT3 | 0.22 | 0.30 | 0.01 | 0.01 | 55 |
| NM_003511.2 | H2AFI | 0.20 | 0.25 | 0.04 | 0.02 | 56 |
| BC017356.1 | IGHM | 1.81 | 1.53 | 0.00 | 0.01 | 78 |
| AB007950.2 | KIAA0481 | 1.03 | 1.05 | 0.02 | 0.01 | 79 |
| X17263.1 | IGKV1D-12 | 0.93 | 0.94 | 0.04 | 0.04 | 81 |
| U65404.1 | KLF1 | 0.62 | 0.54 | 0.03 | 0.04 | 87 |
| K03195.1 | SLC2A1 | 0.29 | 0.25 | 0.03 | 0.00 | 90 |

Table 3: Significant reduced gene activities in samples of patients with sepsis according to [1], represented as their relative relationship to the corresponding gene activities of the same patients in the condition of SIRS according to [1].

TABLE 3

| GenBank | | | | | | |
|---|---|---|---|---|---|---|
| Accession | | mean: | mean: | p: | p: | |
| Number | HUGO-Name | Cy5 vs Cy3 | Cy3 vs Cy5 | Cy5 vs Cy3 | Cy3 vs Cy5 | Seq.-ID |
| NM_000576.1 | IL1B | −0.21 | −0.22 | 0.05 | 0.00 | 58 |
| NM_003022.1 | SH3BGRL | −0.26 | −0.31 | 0.01 | 0.00 | 61 |
| NM_000581.1 | GPX1 | −0.26 | −0.32 | 0.01 | 0.00 | 62 |
| NM_016274.1 | LOC51177 | −0.30 | −0.29 | 0.02 | 0.05 | 63 |
| BC013980.1 | BOP1 | −0.30 | −0.23 | 0.01 | 0.04 | 64 |
| X00457.1 | HLA-DPA1 | −0.31 | −0.21 | 0.01 | 0.04 | 65 |
| NM_001671.2 | ASGR1 | −0.38 | −0.41 | 0.03 | 0.03 | 66 |
| NM_000072.1 | CD36 | −0.38 | −0.38 | 0.02 | 0.02 | 67 |
| BC005943.1 | LOC55974 | −0.42 | −0.30 | 0.02 | 0.01 | 68 |
| NM_004331.1 | BNIP3L | −0.44 | −0.35 | 0.01 | 0.01 | 69 |
| NM_002925.2 | RGS10 | −0.49 | −0.40 | 0.00 | 0.00 | 70 |
| NM_002923.1 | RGS2 | −0.55 | −0.67 | 0.03 | 0.02 | 71 |
| J03041.1 | HLA-DPB1 | −0.56 | −0.51 | 0.00 | 0.01 | 72 |
| NM_000239.1 | LYZ | −0.57 | −0.64 | 0.02 | 0.02 | 73 |
| NM_000345.2 | SNCA | −0.65 | −0.61 | 0.03 | 0.02 | 74 |
| NM_000358.1 | TGFBI | −0.75 | −0.66 | 0.01 | 0.02 | 76 |
| NM_000184.1 | HBG2 | −0.94 | −0.84 | 0.03 | 0.05 | 77 |

These changes characterized in Tables 2 and 3 can be used for the inventive process according to claim 1.

The GenBank Acccession Numbers indicated in Tables 2 and 3 (Internet-access via http://www.ncbi.nlm.nih.gov/) of the individual sequences associated with the attached 42-page sequence protocol of the present application, which is therewith part of the invention, itemized or in detail with respectively one sequence (Sequence ID: 1 up through Sequence ID: 91). This sequence protocol is part of the present invention.

### REFERENCES

1. Bone R C, Balk R A, Cerra F B, Dellinger E P, Fein A M, Knaus W A, Schein R M, Sibbald W J, the ACCP/SCCM Consensus Conference Committee (1992)

Definitions for Sepsis and organ failure and guidelines for the use of innovative therapies in Sepsis. Chest 101, 1656-1662; und Crit Care Med 1992; 20: 864-874.
2. Marshall J C, Vincent J L, Fink M P, Cook D J, Rubenfeld G, Foster D, Fisher C J Jr, Faist E, Reinhart K (2003) Measures, markers, and mediators: toward a staging system for clinical Sepsis. A report of the Fifth Toronto Sepsis Roundtable, Toronto, Ontario, Canada, Oct. 25-26, 2000. Crit Care Med. 31:1560-7.

3. Alberti C, Brun-Buisson C, Goodman S V, Guidici D, Granton J, Moreno R, Smithies M, Thomas O, Artigas A, Le Gall JR; European Sepsis Group (2003) Influence of systemic inflammatory response syndrome and Sepsis on outcome of critically ill infected patients. Am J Respir Crit Care Med. 168:77-84.
4. Bnin-Buisson C, Doyon F, Carlet J, Dellamonica P, Gouin F, Lepoutre A, Mercier J C, Offenstadt G, Regnier B: Incidence, risk factors, and outcome of severe Sepsis and septic shock in adults. A multicenter prospective study in intensive care units. French ICU Group for Severe Sepsis. JAMA 1995; 274: 968-974
5. Le-Gall J R, Lemeshow S, Leleu G, Klar J, Huillard J, Rue M, Teres D, Artigas A: Customized probability models for early severe Sepsis in adult intensive care patients. Intensive Care Unit Scoring Group. JAMA 1995; 273: 644-650
6. Brun-Buisson C, Roudot-Thoraval F, Girou E, Grenier-Sennelier C, Durand-Zaleski I. (2003) The costs of septic syndromes in the intensive care unit and influence of hospital-acquired Sepsis. Intensive Care Med. [Epub ahead of print]
7. Increase in National Hospital Discharge Survey rates for septicemia—United States, 1979-1987. MMWR Morb Mortal Wkly Rep 1990; 39: 31-34
8. Bone, R. C. Sepsis, the sepsis syndrome, multi-organ failure: a plea for comparable definitions. Ann Intern Med 1991; 114: 332-333
9. Matot, I., C. L. Sprung, et al. Definition of sepsis. Intensive Care Med 2001; 27 (suppl): S3-S9.
10. Friedland, J. S., J. C. Porter, et al. Plasma proinflammatory cytokine concentrations, Acute Physiology and Chronic Health Evaluation (APACHE) III scores and survival in patients in an intensive care unit. Crit Care Med 1996; 24: 1775-1781.
11. Beutler, B., A. Poltorak, et al. Sepsis and evolution of the innate immune response. Crit Care Med 2001; 29: S2-S6.
12. Vincent J L, Angus D, Annane D, et al. (2001) Clinical expert round table discussion (session 5) at the Margaux Conference on Critical Illness: outcomes of clinical trials in Sepsis: lessons learned. Crit Care Med 29:S136-137.
13. Abraham, E., Laterre P. F., et al. Lenercept (p55 tumor necrosis factor receptor fusion protein) in severe sepsis and early septic shock: a randomized, double-blind, placebo-controlled, multicenter phase III trial with 1,342 patients. Crit Care Med 2001; 29: 503-510
14. Abraham, E., Reinhart K., et al. Assessment of the safety of recombinant tissue factor pathway inhibitor in patients with severe sepsis: a multicenter, randomized, placebo-controlled, single-blind, dose escalation study. Crit Care Med 2001; 29: 2081-2089
15. Pittet, D., Harbarth S., et al. Impact of immunomodulating therapy on morbidity in patients with severe sepsis. Am J Respir Crit Care Med 1999; 160: 852-857.
16. Abraham, E., Marshall J. C., et al. Sepsis and mediator-directed therapy: rethinking the target populations. Mediator-directed therapy in sepsis: rethinking the target populations. Toronto, Canada, 31 Oct.-1 Nov. 1998. Mol Med Today 1999; 5: 56-58.40-43.
17. Abraham, E., Raffin T. A. Sepsis therapy trials. Continued disappointment or reason for hope? JAMA 1994; 271: 1876-1878.
18. Zeni F., Freeman B., et al. Anti-inflammatory therapies to treat sepsis and septic shock: a reassessment. Crit Care Med 1997; 25: 1095-1100.
19. Bone, R. C. The pathogenesis of sepsis. Ann Intern Med 1991; 115: 457-469.
20. Marshall J C (2000) SIRS and MODS: What is there relevance to the science and practise of intensive care?, Shock 14:586-589.
21. Vincent J-L (1997) Dear SIRS, I'm sorry to say that I don't like you. Crit Car Med 25:372-374.
22. Ramsay G, Gerlach H, Levy M M et al (2003) An international sepsis survey: As tudy of doctor's knowledge and perception about sepsis. Crit Care Med 31.
23. Levy M M, Fink M P, Marshall J C et al. (2003) 2001 SCCM/ESICM/ACCP/ATS/SIS International Sepsis Definitions Conference. Cri Car Med Vol 31, No 4.
24. http://www.krebsinformation.de/tnin-system.lhtml (Stand 1. Marz 2004).
25. Straube E (2003) Sepsis—microbiological diagnosis. Infection 31:284.
26. Ruβwurm S. (2002) Procalcitonin als Marker bakterieller Infektionen und Sepsis: Einfluss sepsisrelevanter Bedingungen auf die Expression von Procalcitonin, Habilitationsschrift eingereicht bei der Medizinischen Fakultat der Friedrich-Schiller-Universitat Jena.
27. Southern E M (1974) An improved method for transferring nucleotides from electrophoresis strips to thin layers of ion-exchange cellulose. Anal Biochem 62:317-318.
28. Gillespie D, Spiegelman S (1965) A quantitative assay for DNA-RNA hybrids with DNA immobilized on a membrane. J Mol Biol 12:829-842.
29. Lennon G G, Lehrach H (1991) Hybridization analyses of arrayed cDNA libraries. Trends Genet 7: 314-317.
30. Kafatos F C, Jones C W, Efstratiadis A (1979) Determination of nucleic acid sequence homologies and relative concentrations by a dot hybridization procedure. Nucl Acid Res 7:1541-1552.
31. Fodor S P, Read J L, Pirrung M C, Stryer L, Lu A T, Solas D (1991) Light-directed, spatially addressable parallel chemical synthesis. Science 251:767-773.
32. Pease A C, Solas D, Sullivan E J, Cronin M T, Holmes C P, Fodor SP (1994) Light-generated oligonucleotide arrays for rapid DNA sequence analysis. Proc Natl Acad Sci USA 91:5022-5026.
33. Schena M, Shalon D, Davis R W, Brown P O (1995) Quantitative monitoring of gene expression patterns with a complementary DNA microarray. Science 270:467-470.
34. Golub T R, Slonim D K, Tamayo P, et al. (1999) Molecular classification of cancer: class discovery and class prediction by gene expression monitoring. Science 286:531-537.
35. Alizadeh A A, Eisen M B, Davis R E, et al. (2000) Distinct types of diffuse large B-cell lymphoma identified by gene expression profiling. Nature 403:503-511.
36. Henry G L, Zito K, Dubnau J, (2003) Chipping away at brain function: mining for insights with microarrays. Current Opinion in Neurobiology, 13:570-576.
37. Fillion I, Ouellet N, Simard M, et al. (2002) Role of chemokines and formyl peptides in pneumococcal pneumonia-induced monocyte/macrophage recruitment. J. Immunol.; 166(12):7353-61.
38. Zhao B, Bowden R A, Stavchansky S A, Bowman P D (2001) Human endothelial cell response to gram-negative lipopolysaccharide assessed with cDNA microarrays. Am J Physiol Cell Physiol. November; 281(5):C 1587-95.
39. Chinnaiyan A M, Huber-Lang M, Kumar-Sinha C et al. (2001) Molecular signatures of Sepsis: multiorgan gene expression profiles of systemic inflammation. Am J. Pathol. 159(4): 1199-209.
40. Cobb J P, Laramie J M, Stormo G D et al. (2002) Sepsis gene expression profiling: Murine splenic compared with hepatic response determined by using complementary DNA microarrays. Crit Care Med Vol. 30, No. 12, 2711-2721.
41. Pathan N, Hemingway C A, Alizadeh A A, et al. (2004) Role of interleukine 6 in myocardial dysfunction of meningococcal septic shock. The Lancet Vol. 363 Nr. 9404: 203-209.
42. Eiling K, Kotsch K, Strohmeyer J-C et al. (2003) Identification of differentially expressed genes during systemic inflammatory response syndrome using cDNA microarrays. Infection 31:301.
43. Huber W, Heydebreck A, Sueltmann H, et al. (2003) Parameter estimation for the calibration and variance stabilization of microarray data. Stat. Appl. in Gen. and Mol. Biol. Volume 2: No 1, Article 3.

1. A method for the in vitro differentiation between systemic inflammatory non-infectious conditions and systemic inflammatory infectious conditions, thereby characterized, that it includes the following steps:
a) isolation of sample RNA from a biological sample;
b) marking the sample RNA and/or at least one DNA, which has a gene activity that is specific for distinguishing between SIRS and sepsis and/or is a specific gene or gene fragment, with a detectable marker;
c) bringing the sample RNA in contract with the DNA in hybridization conditions;
d) bringing control RNA in contact with at least one DNA, under hybridization condition, said DNA representing a gene or gene fragment that is specific for distinguishing between SIRS and sepsis;
e) quantitatively measuring the marking signals of the hybridized sample A and control RNA; and
f) comparing the quantitative data of the marking signals in order to make a determination as to whether genes or gene fragments that are specific for distinguishing between SIRS and sepsis are expressed more prominently or less prominently in the sample than in the control RNA.

2. The process according to claim 1, thereby characterized, that prior to measurement of the sample-RNA the control-RNA is hybridized with the DNA and the marker signal of the control-RNA/DNA-complex is determined and is optionally recorded in the form of a calibration curve or table.

3. The process according to claim 2, thereby characterized, that unchanged gene from the sample and/or control-RNA was used as the reference gene for quantification.

4. The process according to claim 1, thereby characterized, that mRNA was used as the sample-RNA.

5. The process according to claim 1, thereby characterized, that the DNA is provided in predetermined locations, in particular immobilized upon a carrier in the form of a microarray.

6. The process according to claim 1, thereby characterized, that the process is employed for differential diagnostic early recognition, for control of the clinical treatment, for individual risk assessment for patients for evaluation of the probability of response to the specific treatment as well as for post-mortem diagnosis for distinguishing between SRIS and sepsis.

7. The process according to claim 1, thereby characterized, that the sample is selected from: body fluids, in particular blood, serum, urine peritoneal fluid, seminal fluid, saliva, tissue fluids; cell contents or a mixture thereof.

8. The process according to claim 7, thereby characterized, that cell samples are in certain cases subject to lysis treatment in order to release cellular contents.

9. The process according to claim 1, thereby characterized, that the sample is a biological sample of human origin.

10. The process according to claim 1, thereby characterized, that for distinction between SIRS and sepsis, specific gene and/or gene fragments are selected from the group consisting of SEQ-ID No. 1 through SEQ-ID No. 91, as well as gene fragments thereof with at least 5-2000, preferably 20-200, more preferably 20-80 nucleotides.

11. The process according to claim 1, thereby characterized, that at least 2-100 cDNAs are employed.

12. The process according to claim 1, thereby characterized, that at least 200 cDNAs are employed.

13. The process according to claim 1, thereby characterized, that at least 200-500 cDNAs are employed.

14. The process according to claim 1, thereby characterized, that at least 500-1000 cDNAs are employed.

15. The process according to claim 1, thereby characterized, that at least 1000-2000 cDNAs are employed.

16. The process according to claims 1 or 10, thereby characterized, that the genes or gene fragments and/or sequences derived from their RNA listed in claim 10 are replaced by synthetic analogs, aptamers as well as peptido-nucleic acids.

17. The process according to claim 16, thereby characterized, that the synthetic analogs of the genes include 5-100, in particular approximately 70 base pairs.

18. The process according to claim 1, thereby characterized, that the detectable markers are a radioactive marker, in particular ³²P, ¹⁴C, ¹²⁵I, ¹⁵⁵Ep, ³³P, or ³H.

19. The process according to claim 1, thereby characterized, that as the detectable markers a non-radioactive marker, in particular a color or fluorescence marker, an enzyme marker or a immuno marker and/or quantum dots or an electric measurable signal in particular potentiometric and/or conductivity and/or capacitance change upon hybridization, is employed.

20. The process according to claim 1 thereby characterized, that the sample-RNA and the control-RNA and/or enzymatic or chemical derivatives carry the same carrier markers.

21. The process according to claim 1, thereby characterized, that the sample-RNA and the control-RNA and/or enzymatic or chemical derivatives carry different markers.

22. The process according to claim 1, thereby characterized, that the immobilized or non-immobilized samples carry a marker.

23. The process according to claim 1, thereby characterized, that the DNA sample is immobilized on glass or plastic.

24. The process according to claim 1, thereby characterized, that the individual DNA molecules are immobilized by a covalent bonding to the carrier material.

25. The process according to claim 1, thereby characterized, that the individual DNA molecules are immobilized by electrostatic and/or dipol-dipol and/or hydrophobic interaction and/or hydrogen bridges to the carrier materials.

26. Use of recombinant or synthetic produced nucleic acid sequences having specificity for distinguishing between SIRS and sepsis, or partial sequences individually or in subsets, as calibrators in sepsis-assays and/or for evaluation of the effectivity or toxicity in a screening for active substances and/or for production of therapeutics and of substances and substance mixtures, which can be used as therapeutics, for prevention and treatment between SIRS and sepsis.

27. Use of RNA of the gene and/or gene fragment according to claim 10 for obtaining qualitative information regarding the gene activity by hybridization-independent processes, in particular enzymatic and/or chemical hydrolysis and/or amplification processes, preferably PCR, subsequent quantification of the nucleic acids and/or of derivatives and/or fragments thereof.

28. Use of gene activities of genes and/or gene fragments according to claim 10 which are specific for SIRS or sepsis for active substance screening in model organisms.

29. Use of gene activities according to claim 1 which on the cellular level are modulated by gene activities of the genes and/or gene fragments according to claim 10.

30. Use of genes and/or gene fragments according to claim 10 for obtaining information regarding a sepsis or SIRS condition for electronic further processing.

31. Use of gene activity data for the production of software for diagnostic purposes and/or patient data management systems.

32. Use of gene activity data for provision of experimental systems for modeling of cellular signal transmitter pathways.
